SEQUENCE LISTING (209 sequences)

SEQ ID NO: 1 - DNA, 22 bp, Homo sapiens
tgaggtagtagtttgtacagtt

SEQ ID NO: 2 - DNA, 22 bp, Homo sapiens
ctttcagtcggatgtttacagc

SEQ ID NO: 3 - DNA, 22 bp, Homo sapiens
taacactgtctggtaacgatgt

SEQ ID NO: 4 - DNA, 22 bp, Homo sapiens
tgaggtaggaggttgtatagtt

SEQ ID NO: 5 - DNA, 22 bp, Homo sapiens
tgtaaacatcctcgactggaag

SEQ ID NO: 6 - DNA, 22 bp, Homo sapiens
ttccctttgtcatcctatgcct

SEQ ID NO: 7 - DNA, 22 bp, Homo sapiens
ttcaagtaatccaggataggct

SEQ ID NO: 8 - DNA, 22 bp, Homo sapiens
tgaggtagtagtttgtgctgtt

SEQ ID NO: 9 - DNA, 21 bp, Homo sapiens
tacagtactgtgataactgaa

SEQ ID NO: 10 - DNA, 23 bp, Homo sapiens
taatactgccgggtaatgatgga

SEQ ID NO: 11 - DNA, 22 bp, Homo sapiens
ttcaagtaatccaggataggct

SEQ ID NO: 12 - DNA, 22 bp, Homo sapiens
tgaggtagtagtttgtgctgtt

SEQ ID NO: 13 - DNA, 22 bp, Homo sapiens
aaaagctgggttgagagggcga

SEQ ID NO: 14 - DNA, 24 bp, Homo sapiens
tttggcaatggtagaactcacact

SEQ ID NO: 15 - DNA, 22 bp, Homo sapiens
ttccctttgtcatcctatgcct

SEQ ID NO: 16 - DNA, 21 bp, Homo sapiens
tacagtactgtgataactgaa

SEQ ID NO: 17 - DNA, 23 bp, Homo sapiens
taatactgccgggtaatgatgga

SEQ ID NO: 18 - DNA, 22 bp, Homo sapiens
tgaggtagtaggttgtgtggtt

SEQ ID NO: 19 - DNA, 22 bp, Homo sapiens
tgaggtagtagattgtatagtt

SEQ ID NO: 20 - DNA, 23 bp, Homo sapiens
taccctgtagaaccgaatttgtg

SEQ ID NO: 21 - DNA, 22 bp, Homo sapiens
tgaggtaggaggttgtatagtt

SEQ ID NO: 22 - DNA, 22 bp, Homo sapiens
tgtaaacatcctacactcagct

SEQ ID NO: 23 - DNA, 22 bp, Homo sapiens
ttcaagtaatccaggataggct

SEQ ID NO: 24 - DNA, 23 bp, Homo sapiens
taccctgtagatccgaatttgtg

SEQ ID NO: 25 - DNA, 22 bp, Homo sapiens
caaagaattctccttttgggct

SEQ ID NO: 26 - DNA, 22 bp, Homo sapiens
aaaagctgggttgagagggcga

SEQ ID NO: 27 - DNA, 24 bp, Homo sapiens
tccctgagaccctttaacctgtga

SEQ ID NO: 28 - DNA, 24 bp, Homo sapiens
tccctgagaccctttaacctgtga

SEQ ID NO: 29 - DNA, 23 bp, Homo sapiens
aacattcattgctgtcggtgggt

SEQ ID NO: 30 - DNA, 22 bp, Homo sapiens
aagctgccagttgaagaactgt

SEQ ID NO: 31 - DNA, 22 bp, Homo sapiens
cactagattgtgagctcctgga

SEQ ID NO: 32 - DNA, 22 bp, Homo sapiens
aaaagctgggttgagagggcga

SEQ ID NO: 33 - DNA, 22 bp, Homo sapiens
ctttcagtcggatgtttacagc

SEQ ID NO: 34 - DNA, 24 bp, Homo sapiens
tccctgagaccctttaacctgtga

SEQ ID NO: 35 - DNA, 22 bp, Homo sapiens
agaggtagtaggttgcatagtt

SEQ ID NO: 36 - DNA, 21 bp, Homo sapiens
ctgacctatgaattgacagcc

SEQ ID NO: 37 - DNA, 22 bp, Homo sapiens
tgaggtagtagtttgtgctgtt

SEQ ID NO: 38 - DNA, 22 bp, Homo sapiens
tgaggtaggaggttgtatagtt

SEQ ID NO: 39 - DNA, 23 bp, Homo sapiens
aacattcattgctgtcggtgggt

SEQ ID NO: 40 - DNA, 22 bp, Homo sapiens
aaaagctgggttgagagggcga

SEQ ID NO: 41 - DNA, 23 bp, Homo sapiens
taccctgtagaaccgaatttgtg

SEQ ID NO: 42 - DNA, 22 bp, Homo sapiens
tgaggtagtaggttgtatagtt

SEQ ID NO: 43 - DNA, 21 bp, Homo sapiens
cggcggggacggcgattggtc

SEQ ID NO: 44 - DNA, 24 bp, Homo sapiens
tctgggcacaggcggatggacagg

SEQ ID NO: 45 - DNA, 22 bp, Homo sapiens
tattgcactcgtcccggcctcc

SEQ ID NO: 46 - DNA, 22 bp, Homo sapiens
atcgggaatgtcgtgtccgccc

SEQ ID NO: 47 - DNA, 22 bp, Homo sapiens
gactatagaactttccccctca

SEQ ID NO: 48 - DNA, 24 bp, Homo sapiens
tgaagcgcctgtgctctgccgaga

SEQ ID NO: 49 - DNA, 22 bp, Homo sapiens
tccctgagaccctaacttgtga

SEQ ID NO: 50 - DNA, 20 bp, Homo sapiens
cggctctgggtctgtgggga

SEQ ID NO: 51 - DNA, 22 bp, Homo sapiens
tagcttatcagactgatgttga

SEQ ID NO: 52 - DNA, 22 bp, Homo sapiens
tggagtgtgacaatggtgtttg

SEQ ID NO: 53 - DNA, 22 bp, Homo sapiens
tgcaacgaacctgagccactga

SEQ ID NO: 54 - DNA, 23 bp, Homo sapiens
ttaatgctaatcgtgataggggt

SEQ ID NO: 55 - DNA, 21 bp, Homo sapiens
ctttttgcggtctgggcttgc

SEQ ID NO: 56 - DNA, 24 bp, Homo sapiens
tttggcaatggtagaactcacact

SEQ ID NO: 57 - DNA, 19 bp, Homo sapiens
aatggatttttggagcagg

SEQ ID NO: 58 - DNA, 22 bp, Homo sapiens
aaaagctgggttgagagggcaa

SEQ ID NO: 59 - DNA, 22 bp, Homo sapiens
tcggatccgtctgagcttggct

SEQ ID NO: 60 - DNA, 23 bp, Homo sapiens
tctttggttatctagctgtatga

SEQ ID NO: 61 - DNA, 22 bp, Homo sapiens
tgagacctctgggttctgagct

SEQ ID NO: 62 - DNA, 22 bp, Homo sapiens
aactggccctcaaagtcccgct

SEQ ID NO: 63 - DNA, 22 bp, Homo sapiens
tgccctgtggactcagttctgg

SEQ ID NO: 64 - RNA, 22 nt, Homo sapiens
ugagguaguagauuguauaguu

SEQ ID NO: 65 - RNA, 22 nt, Homo sapiens
uccuguacugagcugccccgag

SEQ ID NO: 66 - RNA, 22 nt, Homo sapiens
uggagugugacaaugguguuug

SEQ ID NO: 67 - RNA, 22 nt, Homo sapiens
uauugcacucgucccggccucc

SEQ ID NO: 68 - RNA, 22 nt, Homo sapiens
ugagguaguaguuuguacaguu

SEQ ID NO: 69 - RNA, 21 nt, Homo sapiens
cuagacugaagcuccuugagg

SEQ ID NO: 70 - RNA, 23 nt, Homo sapiens
agcucggucugaggccccucagu

SEQ ID NO: 71 - RNA, 21 nt, Homo sapiens
ucacagugaaccggucucuuu

SEQ ID NO: 72 - RNA, 21 nt, Homo sapiens
cauuauuacuuuugguacgcg

SEQ ID NO: 73 - RNA, 22 nt, Homo sapiens
uucaaguaauccaggauaggcu

SEQ ID NO: 74 - RNA, 21 nt, Homo sapiens
ugauauguuugauauuggguu

SEQ ID NO: 75 - RNA, 23 nt, Homo sapiens
ucggggaucaucaugucacgaga

SEQ ID NO: 76 - RNA, 22 nt, Homo sapiens
uaacacugucugguaacgaugu

SEQ ID NO: 77 - RNA, 22 nt, Homo sapiens
uuugggacugaucuugaugucu

SEQ ID NO: 78 - RNA, 22 nt, Homo sapiens
aagcugccaguugaagaacugu

SEQ ID NO: 79 - RNA, 22 nt, Homo sapiens
ugauauguuugauauauuaggu

SEQ ID NO: 80 - RNA, 22 nt, Homo sapiens
cagugcaaugaugaaagggcau

SEQ ID NO: 81 - RNA, 22 nt, Homo sapiens
ugaucaggcaaaauugcagacu

SEQ ID NO: 82 - RNA, 22 nt, Homo sapiens
cagcagcaauucauguuuugaa

SEQ ID NO: 83 - RNA, 24 nt, Homo sapiens
uguggacagugagguagagggagu

SEQ ID NO: 84 - RNA, 23 nt, Homo sapiens
ucucacacagaaaucgcacccgu

SEQ ID NO: 85 - RNA, 19 nt, Homo sapiens
uaaggagggggaugagggg

SEQ ID NO: 86 - RNA, 22 nt, Homo sapiens
acucggcguggcgucggucgug

SEQ ID NO: 87 - RNA, 22 nt, Homo sapiens
ucacaagucaggcucuugggac

SEQ ID NO: 88 - RNA, 19 nt, Homo sapiens
cuggggaguggcuggggag

SEQ ID NO: 89 - RNA, 25 nt, Homo sapiens
aggggggcacugcgcaagcaaagcc

SEQ ID NO: 90 - RNA, 22 nt, Homo sapiens
agcuuuugggaauucagguagu

SEQ ID NO: 91 - RNA, 22 nt, Homo sapiens
ucacaagucaggcucuugggac

SEQ ID NO: 92 - RNA, 23 nt, Homo sapiens
uuagaggcuggaauagagauucu

SEQ ID NO: 93 - RNA, 22 nt, Homo sapiens
cacgcucaugcacacacccaca

SEQ ID NO: 94 - RNA, 22 nt, Homo sapiens
ugagaccaggacuggaugcacc

SEQ ID NO: 95 - RNA, 23 nt, Homo sapiens
ugaggauggauagcaaggaagcc

SEQ ID NO: 96 - RNA, 20 nt, Homo sapiens
ccucugggcccuuccuccag

SEQ ID NO: 97 - RNA, 22 nt, Homo sapiens
aaugcaccugggcaaggauuca

SEQ ID NO: 98 - RNA, 18 nt, Homo sapiens
cgggcguggugguggggg

SEQ ID NO: 99 - RNA, 23 nt, Homo sapiens
agguuacccgagcaacuuugcau

SEQ ID NO: 100 - RNA, 23 nt, Homo sapiens
aagugccgccaucuuuugagugu

SEQ ID NO: 101 - RNA, 23 nt, Homo sapiens
cccaguguucagacuaccuguuc

SEQ ID NO: 102 - RNA, 22 nt, Homo sapiens
accaucgaccguugauuguacc

SEQ ID NO: 103 - RNA, 22 nt, Homo sapiens
aagcugccaguugaagaacugu

SEQ ID NO: 104 - RNA, 21 nt, Homo sapiens
gcaguccaugggcauauacac

SEQ ID NO: 105 - RNA, 22 nt, Homo sapiens
aaagugcuucccuuuggacugu

SEQ ID NO: 106 - RNA, 22 nt, Homo sapiens
cagugcaaugaugaaagggcau

SEQ ID NO: 107 - RNA, 23 nt, Homo sapiens
ugaggauggauagcaaggaagcc

SEQ ID NO: 108 - RNA, 23 nt, Homo sapiens
agguugggaucgguugcaaugcu

SEQ ID NO: 109 - RNA, 22 nt, Homo sapiens
cugcccuggcccgagggaccga

SEQ ID NO: 110 - RNA, 23 nt, Homo sapiens
ccucagggcuguagaacagggcu

SEQ ID NO: 111 - RNA, 22 nt, Homo sapiens
cugcccuggcccgagggaccga

SEQ ID NO: 112 - RNA, 23 nt, Homo sapiens
uauggcuuuucauuccuauguga

SEQ ID NO: 113 - RNA, 22 nt, Homo sapiens
gcucugacuuuauugcacuacu

SEQ ID NO: 114 - RNA, 22 nt, Homo sapiens
aaucauacacgguugaccuauu

SEQ ID NO: 115 - RNA, 22 nt, Homo sapiens
gcugcgcuuggauuucgucccc

SEQ ID NO: 116 - RNA, 23 nt, Homo sapiens
accuuggcucuagacugcuuacu

SEQ ID NO: 117 - RNA, 22 nt, Homo sapiens
cugggagaggguuguuuacucc

SEQ ID NO: 118 - RNA, 22 nt, Homo sapiens
cugggagaggguuguuuacucc

SEQ ID NO: 119 - RNA, 20 nt, Homo sapiens
acugccccaggugcugcugg

SEQ ID NO: 120 - RNA, 21 nt, Homo sapiens
uucacaguggcuaaguucugc

SEQ ID NO: 121 - RNA, 22 nt, Homo sapiens
acuggacuuggagucagaaggc

SEQ ID NO: 122 - RNA, 22 nt, Homo sapiens
uucaaguaauccaggauaggcu

SEQ ID NO: 123 - RNA, 21 nt, Homo sapiens
uaccacaggguagaaccacgg

SEQ ID NO: 124 - RNA, 22 nt, Homo sapiens
uagcagcacguaaauauuggcg

SEQ ID NO: 125 - RNA, 22 nt, Homo sapiens
caaagaauucuccuuuugggcu

SEQ ID NO: 126 - RNA, 22 nt, Homo sapiens
cuuucagucggauguuuacagc

SEQ ID NO: 127 - RNA, 22 nt, Homo sapiens
cauugcacuugucucggucuga

SEQ ID NO: 128 - RNA, 22 nt, Homo sapiens
caugccuugaguguaggaccgu

SEQ ID NO: 129 - RNA, 22 nt, Homo sapiens
uuaugguuugccugggacugag

SEQ ID NO: 130 - RNA, 23 nt, Homo sapiens
agcagcauuguacagggcuauga

SEQ ID NO: 131 - RNA, 22 nt, Homo sapiens
ugagaacugaauuccauggguu

SEQ ID NO: 132 - RNA, 22 nt, Homo sapiens
cuuucagucggauguuuacagc

SEQ ID NO: 133 - DNA, 15 bp, Artificial Sequence
  inserted to correct for sequence numbering
aaaaaaaaaaaaaaa

SEQ ID NO: 134 - RNA, 22 nt, Homo sapiens
uucaaguaauccaggauaggcu

SEQ ID NO: 135 - RNA, 23 nt, Homo sapiens
acugcccuaagugcuccuucugg

SEQ ID NO: 136 - RNA, 22 nt, Homo sapiens
aagcugccaguugaagaacugu

SEQ ID NO: 137 - RNA, 23 nt, Homo sapiens
agcucggucugaggccccucagu

SEQ ID NO: 138 - RNA, 21 nt, Homo sapiens
cuagacugaagcuccuugagg

SEQ ID NO: 139 - RNA, 23 nt, Homo sapiens
ugugcuugcucgucccgcccgca

SEQ ID NO: 140 - RNA, 22 nt, Homo sapiens
cacuagauugugagcuccugga

SEQ ID NO: 141 - RNA, 23 nt, Homo sapiens
ucccccaggugugauucugauuu

SEQ ID NO: 142 - RNA, 22 nt, Homo sapiens
ucaggcucaguccccucccgau

SEQ ID NO: 143 - RNA, 22 nt, Homo sapiens
acuggacuuggagucagaaggc

SEQ ID NO: 144 - RNA, 22 nt, Homo sapiens
cuggcccucucugcccuuccgu

SEQ ID NO: 145 - RNA, 22 nt, Homo sapiens
acuggacuuggagucagaaggc

SEQ ID NO: 146 - RNA, 22 nt, Homo sapiens
uagcagcacaucaugguuuaca

SEQ ID NO: 147 - RNA, 22 nt, Homo sapiens
uauggcacugguagaauucacu

SEQ ID NO: 148 - RNA, 23 nt, Homo sapiens
acugcccuaagugcuccuucugg

SEQ ID NO: 149 - RNA, 21 nt, Homo sapiens
aauggcgccacuaggguugug

SEQ ID NO: 150 - RNA, 21 nt, Homo sapiens
aucacauugccagggauuacc

SEQ ID NO: 151 - RNA, 21 nt, Homo sapiens
uucacaguggcuaaguucugc

SEQ ID NO: 152 - RNA, 24 nt, Homo sapiens
uuuggcaaugguagaacucacacu

SEQ ID NO: 153 - RNA, 21 nt, Homo sapiens
uaccacaggguagaaccacgg

SEQ ID NO: 154 - RNA, 22 nt, Homo sapiens
aaugcacccgggcaaggauucu

SEQ ID NO: 155 - DNA, 21 bp, Artificial Sequence
  inserted to correct for sequence numbering
aaaaaaaaaaaaaaaaaaaaa

SEQ ID NO: 156 - RNA, 22 nt, Homo sapiens
cuuucagucggauguuuacagc

SEQ ID NO: 157 - RNA, 24 nt, Homo sapiens
ugaagcgccugugcucugccgaga

SEQ ID NO: 158 - RNA, 21 nt, Homo sapiens
cugaccuaugaauugacagcc

SEQ ID NO: 159 - RNA, 22 nt, Homo sapiens
aaugcaccugggcaaggauuca

SEQ ID NO: 160 - RNA, 22 nt, Homo sapiens
uguaaacauccccgacuggaag

SEQ ID NO: 161 - RNA, 20 nt, Homo sapiens
acugccccaggugcugcugg

SEQ ID NO: 162 - RNA, 22 nt, Homo sapiens
ucagugcacuacagaacuuugu

SEQ ID NO: 163 - RNA, 21 nt, Homo sapiens
aucacauugccagggauuucc

SEQ ID NO: 164 - RNA, 23 nt, Homo sapiens
agcucggucugaggccccucagu

SEQ ID NO: 165 - RNA, 22 nt, Homo sapiens
cagagucucguucuguugccca

SEQ ID NO: 166 - RNA, 23 nt, Homo sapiens
cacccggcugugugcacaugugc

SEQ ID NO: 167 - RNA, 22 nt, Homo sapiens
cacuagauugugagcuccugga

SEQ ID NO: 168 - RNA, 22 nt, Homo sapiens
ugagguaguagguugugugguu

SEQ ID NO: 169 - RNA, 23 nt, Homo sapiens
ugugcaaauccaugcaaaacuga

SEQ ID NO: 170 - RNA, 20 nt, Homo sapiens
ccucugggcccuuccuccag

SEQ ID NO: 171 - RNA, 21 nt, Homo sapiens
cugaccuaugaauugacagcc

SEQ ID NO: 172 - RNA, 23 nt, Homo sapiens
aacauucaacgcugucggugagu

SEQ ID NO: 173 - RNA, 22 nt, Homo sapiens
uccuguacugagcugccccgag

SEQ ID NO: 174 - RNA, 22 nt, Homo sapiens
ugagguaguagguugugugguu

SEQ ID NO: 175 - RNA, 22 nt, Homo sapiens
cacccguagaaccgaccuugcg

SEQ ID NO: 176 - RNA, 22 nt, Homo sapiens
ugagguaguagguugugugguu

SEQ ID NO: 177 - RNA, 22 nt, Homo sapiens
uauugcacuugucccggccugu

SEQ ID NO: 178 - RNA, 22 nt, Homo sapiens
ugagguaguaguuuguacaguu

SEQ ID NO: 179 - RNA, 22 nt, Homo sapiens
uauugcacucgucccggccucc

SEQ ID NO: 180 - RNA, 21 nt, Homo sapiens
cauuauuacuuuugguacgcg

SEQ ID NO: 181 - RNA, 23 nt, Homo sapiens
ugaggggcagagagcgagacuuu

SEQ ID NO: 182 - RNA, 21 nt, Homo sapiens
cuagacugaagcuccuugagg

SEQ ID NO: 183 - RNA, 23 nt, Homo sapiens
agcucggucugaggccccucagu

SEQ ID NO: 184 - RNA, 21 nt, Homo sapiens
ucacagugaaccggucucuuu

SEQ ID NO: 185 - RNA, 22 nt, Homo sapiens
uagcuuaucagacugauguuga

SEQ ID NO: 186 - RNA, 21 nt, Homo sapiens
ucgaggagcucacagucuagu

SEQ ID NO: 187 - RNA, 21 nt, Homo sapiens
cauaaaguagaaagcacuacu

SEQ ID NO: 188 - RNA, 21 nt, Homo sapiens
aucacauugccagggauuucc

SEQ ID NO: 189 - RNA, 22 nt, Homo sapiens
ugagguaguagauuguauaguu

SEQ ID NO: 190 - RNA, 22 nt, Homo sapiens
aagcugccaguugaagaacugu

SEQ ID NO: 191 - RNA, 22 nt, Homo sapiens
ucucccaacccuuguaccagug

SEQ ID NO: 192 - RNA, 23 nt, Homo sapiens
ugaggggcagagagcgagacuuu

SEQ ID NO: 193 - RNA, 22 nt, Homo sapiens
gcugacuccuaguccagggcuc

SEQ ID NO: 194 - RNA, 22 nt, Homo sapiens
ugagguaguaguuugugcuguu

SEQ ID NO: 195 - RNA, 23 nt, Homo sapiens
aacauucaacgcugucggugagu

SEQ ID NO: 196 - RNA, 21 nt, Homo sapiens
cauuauuacuuuugguacgcg

SEQ ID NO: 197 - RNA, 22 nt, Homo sapiens
uucaaguaauccaggauaggcu

SEQ ID NO: 198 - DNA, 32 bp, Homo sapiens
gtattgcacttgtcccggcctgttgagtttgg

SEQ ID NO: 199 - DNA, 22 bp, Homo sapiens
tattgcacttgtcccggcctgt

SEQ ID NO: 200 - DNA, 23 bp, Artificial Sequence
  modified hsa-miR-92a-3p
tattgcacttgtcccggcctgta

SEQ ID NO: 201 - DNA, 24 bp, Artificial Sequence
  modified hsa-miR-92a-3p
tattgcacttgtcccggcctgtaa

SEQ ID NO: 202 - DNA, 25 bp, Artificial Sequence
  modified hsa-miR-92a-3p
tattgcacttgtcccggcctgtaaa

SEQ ID NO: 203 - DNA, 24 bp, Artificial Sequence
  modified hsa-miR-92a-3p
tattgcacttgtcccggcctgttt

SEQ ID NO: 204 - DNA, 23 bp, Artificial Sequence
  modified hsa-miR-92a-3p
tattgcacttgtcccggcctgtc

SEQ ID NO: 205 - DNA, 23 bp, Artificial Sequence
  modified hsa-miR-92a-3p
tattgcacttgtcccggcctgtg

SEQ ID NO: 206 - DNA, 25 bp, Artificial Sequence
  modified hsa-miR-92a-3p
tattgcacttgtcccggcctgttga

SEQ ID NO: 207 - DNA, 19 bp, Artificial Sequence
  modified hsa-miR-92a-3p
tattgcacttgtcccggcc

SEQ ID NO: 208 - DNA, 23 bp, Artificial Sequence
  modified hsa-miR-92a-3p
gtattgcacttgtcccggcctgt

SEQ ID NO: 209 - DNA, 21 bp, Artificial Sequence
  modified hsa-miR-92a-3p
attgcacttgtcccggcctgt

(19) United States
(12) Patent Application Publication
(10) Pub. No.: US 2017/0051359 A1
(43) Pub. Date: Feb. 23, 2017

(54) SMALL ncRNAS AS BIOMARKERS

(71) Applicants: Stichting VUmc, Amsterdam (NL); Universidad de Granada, Granada (ES)

(72) Inventors: Dirk Michiel Pegtel, Amsterdam (NL); Danijela Koppers-Lalic, Amsterdam (NL); Tom Wurdinger, Amsterdam (NL); Irene Bijnsdorp, Amsterdam (NL); Michael Hackenberg, Granada (ES)

(73) Assignees: Stichting VUmc, Amsterdam (NL); Universidad de Granada, Granada (ES); Universidad de Granada, Granada (ES)

(21) Appl. No.: 15/308,328

(22) PCT Filed: Apr. 21, 2015
(86) PCT No.: PCT/EP2015/058614

§ 371 Date: Nov. 1, 2016

(30) Foreign Application Priority Data
May 1, 2014 (EP) 14166802.0

(51) Int. Cl. C12Q 1/68; A61K 38/14; A61K 31/7048; A61K 31/573; A61K 31/675; A61K 31/475; A61K 31/166; A61K 35/545; A61K 31/704

(52) U.S. Cl. CPC C12Q 1/6886; A61K 35/545; A61K 38/14; A61K 31/7048; A61K 31/704; A61K 31/675; A61K 31/475; A61K 31/166; A61K 31/573; C12Q 2600/178; C12Q 2600/118

(57) ABSTRACT

Disclosed are small ncRNAs that may be used as biomarkers for classifying the health status of an individual. The disclosure also provides screening methods for identifying ncRNA biomarkers.

### CROSS-REFERENCE TO RELATED APPLICATIONS

This application is a national phase entry under 35 U.S.C. §371 of International Patent Application PCT/EP2015/058614, filed Apr. 21, 2015, designating the United States of America and published in English as International Patent Publication WO 2015/165779 A2 on Nov. 5, 2015, which claims the benefit under Article 8 of the Patent Cooperation Treaty to European Patent Application Serial No. 14166802.0, filed May 1, 2014.

### STATEMENT ACCORDING TO 37 C.F.R. §1.821(C) OR (E)—SEQUENCE LISTING SUBMITTED AS ASCII TEXT FILE

Pursuant to 37 C.F.R. §1.821(c) or (e), a file containing an ASCII text version of the Sequence Listing has been submitted concomitant with this application, the contents of which are hereby incorporated by reference.

### TECHNICAL FIELD

The disclosure provides small ncRNAs as biomarkers for classifying the health status of an individual. The disclosure also provides screening methods for identifying ncRNA biomarkers.

### BACKGROUND

The human genome encodes for a vast amount of small non-protein-coding RNA (ncRNAs) transcripts. Multiple ncRNA classes have been described including the highly abundant transfer RNAs (tRNAs), ribosomal RNAs (rRNAs), small nucleolar RNAs (snoRNAs), microRNAs (miRNAs), small interfering RNAs (siRNAs), small nuclear RNAs (snRNAs), and piwi-interacting RNAs (piRNAs) (Amaral et al., 2008; Martens-Uzunova et al., 2013). MiRNAs act as translational repressors by binding to target mRNAs at sites with adequate sequence complementary (Ameres et al., 2007), while the highly abundant cytoplasmic Y RNAs function in RNA quality control by affecting the subcellular location of Ro proteins (Sim et al., 2009). The repressive activity of mature miRNAs on mRNA translation is shared by other classes of ncRNAs, including siRNAs and endo-siRNAs, in addition to piRNAs that silence retrotransposons at defined subcellular locations (Chuma and Pillai, 2009). MiRNA activity relies on sufficient levels of abundance in the cytoplasm, and interaction with RNA-induced silencing complexes (RISC) localized at endosomal membranes (Gibbings et al., 2009; Lee et al., 2009a), whereas low abundant miRNAs have less impact on translational repression. As a consequence, subtle alterations in the levels of certain miRNA may already influence cellular processes, while strong perturbations can cause disease. Abundance, interactions with (RISC) proteins in conjunction with RNA partners, and correct subcellular localization are interrelated factors that control miRNA physiology (Mullokandov et al., 2012; Wee et al., 2012).

MiRNAs are a class of small, 22- to 25-nucleotide, non-coding regulatory RNAs that control key aspects of post-translational gene regulation and function in a highly specific manner. Since miRNAs act as specific gene regulators and because their expression is frequently perturbed in cancer development, their use as biomarkers has been investigated. Overexpression of certain miRNA (oncomirs), such as miR-21, or lack of expression, such as the miR-200 family, seem to correlate with clinically aggressive or metastatic disease outcome. In chronic lymphocytic leukemia (CLL), circulating miRNAs have been used for disease stratification and predicted the response to therapeutic intervention.

Nevertheless, although miRNA profiling is a relatively standard technique, widespread clinical implementation of circulating miRNAs has been hampered due to conflicting data.

### BRIEF SUMMARY

One aspect of the disclosure provides a method for identifying a small non-coding RNA (ncRNA) biomarker pair, the method comprising
for an ncRNA, determining the ratio of two different varieties of ncRNA in a first bodily fluid sample obtained from a first individual reference and in a second bodily fluid sample obtained from a second individual reference, wherein, the first individual reference has an altered health status from the second individual reference,
comparing the ratios from the first and second bodily fluid sample, and
identifying the two different varieties of ncRNA as a biomarker pair when the ratio is altered between the first and second bodily fluid sample,
wherein, the first variety is selected from the canonical ncRNA and the ncRNA with a 3′ non-templated nucleotide addition and the second variety is the ncRNA with a 3′ non-templated nucleotide addition, and
wherein, when ncRNA is miRNA, the first variety is selected from canonical ncRNA, the ncRNA trimmed at the 5′ or 3′ end, and the ncRNA with a 3′ non-templated nucleotide addition, optionally trimmed at the 5′ or 3′ end and the second variety is selected from ncRNA trimmed at the 5′ or 3′ end and ncRNA with a 3′ non-templated nucleotide addition, optionally trimmed at the 5′ or 3′ end.

Alternatively stated,
wherein, the first variety is selected from the canonical ncRNA, the ncRNA trimmed at the 5′ or 3′ end, and the ncRNA with a 3′ non-templated nucleotide addition optionally trimmed at the 5′ or 3′ end;
wherein, the second variety is selected from the ncRNA trimmed at the 5′ or 3′ end and the ncRNA with a 3′ non-templated nucleotide addition optionally trimmed at the 5′ or 3′ end;
wherein, when ncRNA is not an miRNA, the first variety is selected from the canonical ncRNA and the ncRNA with a 3′ non-templated nucleotide addition and the second variety is the ncRNA with a 3′ non-templated nucleotide addition.

Preferably, the first and second varieties are selected from the canonical ncRNA; the ncRNA trimmed at the 5′ or 3′ end and/or extended at the 5′ or 3′ end; and the ncRNA with a 3′ non-templated nucleotide addition, optionally trimmed at the 5′ or 3′ end or extended at the 5′ or 3′ end;

wherein, when ncRNA is not an miRNA, the first variety is selected from the canonical ncRNA and the ncRNA with a 3′ non-templated nucleotide addition and the second variety is the ncRNA with a 3′ non-templated nucleotide addition.

Preferably, the method comprises determining the quantity of two different varieties of ncRNA in a first bodily fluid sample and determining the quantity of two different varieties of ncRNA in a second bodily fluid sample.

Preferably, the first individual reference is from one or more healthy individuals and the second individual reference is from one or more individuals having a disorder, preferably wherein, the disorder is prostate cancer.

Preferably, the first individual reference is from an individual having a disorder and the second individual reference is from the same individual following treatment of the disorder.

In preferred aspects of the methods disclosed herein, the biomarker pair ratio is determined by quantifying the two different varieties in each sample and determining the relationship between the two quantities. Preferably, the varieties are quantified using deep sequencing (RNA-seq).

A further aspect of the disclosure provides for a method for collecting data for classifying the health status of an individual using a small non-coding RNA (ncRNA) biomarker pair. The method comprises determining the ratio of the biomarker pair in a bodily fluid sample from an individual and comparing the ratio to the ratio of the biomarker pair in a bodily fluid from a reference sample (such as the second individual reference described above),
wherein, the presence or absence in a difference in the ratio between the individual and the reference sample assists in classifying the health status of the individual,
wherein, the biomarker pair consists of two different varieties of an ncRNA
wherein, the first variety is selected from the canonical ncRNA and the ncRNA with a 3′ non-templated nucleotide addition and the second variety is the ncRNA with a 3′ non-templated nucleotide addition, and
wherein, when ncRNA is miRNA, the first variety is selected from canonical ncRNA, the ncRNA trimmed at the 5′ or 3′ end, and the ncRNA with a 3′ non-templated nucleotide addition, optionally trimmed at the 5′ or 3′ end and the second variety is selected from ncRNA trimmed at the 5′ or 3′ end and ncRNA with a 3′ non-templated nucleotide addition, optionally trimmed at the 5′ or 3′ end.

Alternatively stated,
wherein, the first variety is selected from the canonical ncRNA, the ncRNA trimmed at the 5′ or 3′ end, and the ncRNA with a 3′ non-templated nucleotide addition optionally trimmed at the 5′ or 3′ end;
wherein, the second variety is selected from the ncRNA trimmed at the 5′ or 3′ end and the ncRNA with a 3′ non-templated nucleotide addition optionally trimmed at the 5′ or 3′ end;
wherein, when ncRNA is not an miRNA, the first variety is selected from the canonical ncRNA and the ncRNA with a 3′ non-templated nucleotide addition and the second variety is the ncRNA with a 3′ non-templated nucleotide addition.

Preferably, the first and second varieties are selected from the canonical ncRNA; the ncRNA trimmed at the 5′ or 3′ end and/or extended at the 5′ or 3′ end; and the ncRNA with a 3′ non-templated nucleotide addition, optionally trimmed at the 5′ or 3′ end or extended at the 5′ or 3′ end;

wherein, when ncRNA is not an miRNA, the first variety is selected from the canonical ncRNA and the ncRNA with a 3′ non-templated nucleotide addition and the second variety is the ncRNA with a 3′ non-templated nucleotide addition.

The data obtained in such a method can be used either alone or in combination with other factors (e.g., the presence of additional biomarkers, clinical symptoms in a patient, etc.) to diagnose the individual as having a disorder.

A further aspect of the disclosure provides for a method for classifying the health status of an individual using a small non-coding RNA (ncRNA) biomarker pair, the method comprising determining the ratio of the biomarker pair in a bodily fluid sample from the individual and comparing the ratio to the ratio of the biomarker pair in a bodily fluid from a reference sample,
wherein, the presence or absence in a difference in the ratio between the individual and the reference sample classifies the health status of the individual,
wherein, the biomarker pair consists of two different varieties of an ncRNA
wherein, the first variety is selected from the canonical ncRNA and the ncRNA with a 3′ non-templated nucleotide addition and the second variety is the ncRNA with a 3′ non-templated nucleotide addition, and
wherein, when ncRNA is miRNA, the first variety is selected from canonical ncRNA, the ncRNA trimmed at the 5′ or 3′ end, and the ncRNA with a 3′ non-templated nucleotide addition, optionally trimmed at the 5′ or 3′ end and the second variety is selected from ncRNA trimmed at the 5′ or 3′ end and ncRNA with a 3′ non-templated nucleotide addition, optionally trimmed at the 5′ or 3′ end.

Alternatively stated,
wherein, the first variety is selected from the canonical ncRNA, the ncRNA trimmed at the 5′ or 3′ end, and the ncRNA with a 3′ non-templated nucleotide addition optionally trimmed at the 5′ or 3′ end;
wherein, the second variety is selected from the ncRNA trimmed at the 5′ or 3′ end and the ncRNA with a 3′ non-templated nucleotide addition optionally trimmed at the 5′ or 3′ end;
wherein, when ncRNA is not an miRNA, the first variety is selected from the canonical ncRNA and the ncRNA with a 3′ non-templated nucleotide addition and the second variety is the ncRNA with a 3′ non-templated nucleotide addition.

Preferably, the first and second varieties are selected from the canonical ncRNA; the ncRNA trimmed at the 5′ or 3′ end and/or extended at the 5′ or 3′ end; and the ncRNA with a 3′ non-templated nucleotide addition, optionally trimmed at the 5′ or 3′ end or extended at the 5′ or 3′ end;
wherein, when ncRNA is not an miRNA, the first variety is selected from the canonical ncRNA and the ncRNA with a 3′ non-templated nucleotide addition and the second variety is the ncRNA with a 3′ non-templated nucleotide addition.

Preferably, the methods comprise determining the quantity of the biomarker pair in the individual sample.

Preferably, the reference sample is from one or more healthy individuals.

Preferably, the reference sample is from one or more individuals having a disorder.

Preferably, the disorder as disclosed herein is cancer, more preferably prostate cancer, breast cancer, cHL, testicular cancer or colorectal cancer. Preferably, the disorder is prostate cancer, breast cancer, or cHL. Preferably, the disorder is Alzheimer's disease.

Preferably, the reference sample is from one or more individuals having a good response or poor response to treatment for a disorder.

In preferred aspects of the methods disclosed herein, the ncRNA is selected from transfer RNA (tRNA), ribosomal RNA (rRNA), snoRNAs, microRNA (miRNA), siRNAs, small nuclear RNA (snRNA), Y RNA, vault RNA, antisense RNA and piwiRNA (piRNA), preferably, wherein, ncRNA is selected from miRNA.

In preferred aspects of the methods disclosed herein, the bodily fluid is urine or blood.

In preferred aspects of the methods disclosed herein, the two different varieties are selected from canonical and non-templated additions (NTA) as follows: canonical and 3′ NTA-A; canonical and 3′ NTA-G; canonical and 3′ NTA-C; canonical and 3′ NTA-U; 3′ NTA-G and 3′ NTA-C; 3′ NTA-G and 3′ NTA-U; 3′ NTA-G and 3′ NTA-A; 3′ NTA-C and 3′ NTA-U; 3′ NTA-C and 3′ NTA-A; and 3′ NTA-U and 3′ NTA-A; canonical and 5′ trimmed; canonical and 3′ trimmed; 5′ trimmed and 3′ NTA-C; 5′ trimmed and 3′ NTA-U; 5′ trimmed and 3′ NTA-A; 5′ trimmed and 3′ NTA-G; 3′ trimmed and 3′ NTA-C; 3′ trimmed and 3′ NTA-U; 3′ trimmed and 3′ NTA-A; 3′ trimmed and 3′ NTA-G; and 3′ trimmed and 5′ trimmed; extended and 3′ NTA-A; extended and 3′ NTA-G; extended and 3′ NTA-C; extended and 3′ NTA-U; extended and 5′ trimmed; extended and 3′ trimmed; 5′ or 3′ trimmed and 5′ or 3′ extended; (trimmed and/or extended) and 3′ NTA-U; (trimmed and/or extended) and 3′ NTA-A; (trimmed and/or extended) and 3′ NTA-G; (trimmed and/or extended) and 3′ NTA-C; (trimmed and/or extended) and canonical.

In preferred aspects of the methods disclosed herein, each ncRNA of the biomarker pair comprises at least 16 nucleotides.

FIGS. 12A-12C provide an exemplary embodiment of biomarker pairs of the disclosure. Table 1 is understood to disclose that one biomarker is the canonical sequence and the other biomarker is the respective 3′ NTA-A variety, e.g., hsa-let-7g-5p canonical and hsa-let-7g-5p 3′ NTA-A. Table 1A thus discloses nine separate biomarker pairs. Table 1B discloses five separate biomarker pairs (e.g., hsa-miR-200c-3p canonical and hsa-miR-200c-3p 3′ NTA-U; Table 1C discloses four separate biomarker pairs (e.g., hsa-miR-204-5p canonical and hsa-miR-204-5p 3′ NTA-C); Table 1D discloses nine separate biomarker pairs (e.g., hsa-let-7f-5p canonical and hsa-let-7f-5p 3′ NTA-G); Table 1E discloses eleven separate biomarker pairs (e.g., hsa-let-7f-5p canonical and hsa-let-7f-5p 3′ trimmed; Table 1F discloses three separate biomarker pairs (e.g., hsa-miR-181b-5p canonical and hsa-miR-181b-5p 5′ trimmed).

In preferred aspects of the methods disclosed herein, the biomarker pair is selected from one of the biomarker pairs depicted in FIGS. 12A-12C. Preferably, the biomarker pair is selected from Table 1A. Preferably, the biomarker pair is selected from Table 1B. Preferably, the biomarker pair is selected from Table 1C. Preferably, the biomarker pair is selected from Table 1D. Preferably, the biomarker pair is selected from Table 1E. Preferably, the biomarker pair is selected from Table 1F.

FIGS. 21A-21S provide further exemplary embodiments of biomarkers of the disclosure. Table 2 depicts tRNAs biomarker pairs useful in characterizing prostate cancer. Table 2 is understood to disclose that one biomarker is the canonical sequence and the other biomarker is the respective variant. In particular, these biomarker pairs are useful in methods that determine the biomarker pairs in urine. Tables 4 and 10 depict miRNA biomarker pairs useful in characterizing cHL. In particular, these biomarker pairs are useful in methods that determine the biomarker pairs in exosomal vesicles extracted from blood. Tables 4 and 10 are understood to disclose that one biomarker is the canonical sequence and the other biomarker is the respective variant. Tables 5 and 11 depict miRNA biomarker pairs useful in characterizing cHL. In particular, these biomarker pairs are useful in methods that determine the biomarker pairs in the protein fraction extracted from blood. Tables 5 and 11 are understood to disclose that one biomarker is the canonical sequence and the other biomarker is the respective variant. Table 6 depicts miRNA biomarker pairs useful in characterizing breast cancer. In particular, these biomarker pairs are useful in methods that determine the biomarker pairs in blood samples. Table 6 is understood to disclose that one biomarker is the canonical sequence and the other biomarker is the respective variant. Table 7 depicts miRNA biomarker pairs useful in characterizing testicular germ cell tumors. Table 7 is understood to disclose that one biomarker is the canonical sequence and the other biomarker is the respective variant. Table 8 depicts miRNA biomarker pairs useful in colorectal cancer. Table 8 is understood to disclose that one biomarker is the canonical sequence and the other biomarker is the respective variant. Table 9 depicts miRNA biomarker pairs useful in Alzheimer's disease. Table 9 is understood to disclose that one biomarker is the canonical sequence and the other biomarker is the respective variant. In particular, these biomarker pairs are useful in methods that determine the biomarker pairs in blood samples.

In preferred aspects of the methods disclosed herein, the biomarker pair is selected from Tables 1, 2, or 4-11, preferably from Tables 1, 2, 4, 5, 6, and 9-11.

Preferably, in the methods disclosed herein, the quantity of the two different varieties of ncRNA are determined from exosomal vesicles purified from the bodily fluid samples. Preferably, in the methods disclosed herein, the quantity of the two different varieties of ncRNA are determined from the protein fraction purified from the bodily fluid samples. Preferably, the exosomal vesicles or protein fraction are purified by subjecting the bodily fluid samples to size-exclusion chromatography (SEC).

In a further aspect of the disclosure, a method is provided for characterizing the status of classical Hodgkin's lymphoma (cHL) in an individual, the method comprising determining the quantity of one or more miRNAs from a bodily fluid sample from the individual and comparing the amount of the one or more miRNAs to a reference sample, wherein, the difference in the presence or amount of the one or more miRNAs characterizes the status of the individual. A method is also provided for collecting data regarding the health status of an individual comprising determining the quantity of one or more miRNAs from a bodily fluid sample from the individual and comparing the amount of the one or more miRNAs to a reference sample. The data can be used for characterizing the status of classical Hodgkin's lymphoma (cHL) in the individual.

The one or more miRNAs used in these methods are selected in Table 3.

Preferably, the one or more miRNAs are selected from miR21-5p, let7a-5p, miR127-3p, and miR155-5p.

Table 3 depicts miRNAs useful in characterizing the status of classical Hodgkin's lymphoma (cHL) in an individual. In particular, these biomarker pairs are useful in methods that determine the biomarker pairs in the protein fraction extracted from blood. Preferably, the method characterizes the status of cHL by determining whether an individual is afflicted with cHL.

Preferably, the reference sample is from one or more healthy individuals.

Preferably, the reference sample is from one or more individuals having cHL.

Preferably, characterizing the status of cHL comprises determining the treatment efficacy in an individual receiving treatment for cHL. Preferably, the reference sample is from the same individual prior to receiving treatment for cHL.

Preferably, characterizing the status of cHL comprises determining the prognosis of an individual afflicted with cHL. Preferably, the reference sample is from one or more individuals having a good response to treatment for the disorder or from one or more individuals having a poor response to treatment for the disorder.

In preferred embodiments, the methods further comprise purifying exosomal vesicles from the bodily fluid sample and determining the quantity of the one or more miRNAs associated with the purified exosomal vesicles. In preferred embodiments, the methods further comprise purifying the protein fraction from the bodily fluid sample and determining the quantity of the one or more miRNAs associated with the purified exosomal vesicles. Preferably, purification comprises subjecting the bodily fluid samples to size-exclusion chromatography (SEC). Preferably, the exosomal vesicles are isolated by obtaining the void volume fraction.

In preferred embodiments, the bodily fluid is blood.

Preferably, the methods disclosed herein are performed in vitro, in particular, the step of quantifying the relevant biomarkers is performed in vitro.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C: Small RNA repertoire from B cells and their exosomes. FIG. 1A) Summary of sample characteristics and RNA-seq data. cDNA libraries were generated from cellular and exosomal small RNA fractions. Total number of reads from all libraries before and after mapping to genome of interest (hsg 19; human genome) is specified. To provide annotations to RNA elements that mapped to human genome, all mapped reads were analyzed against currently known databases: 1) mature and pre-miRNA sequences from miRBase version 19, 2) NCBI Reference Sequences human RefSeq genes downloaded Jun. 2, 2013, 3) tRNA sequences from the genomic tRNA database, 4) Repeat Derived sequences detected by means of the RepeatMasker algorithm downloaded from UCSC and 5) piwiRNA (piRNAs) downloaded from the NCBI nucleotide database. FIG. 1B) cDNA libraries were generated from cellular and exosomal small RNA fractions. Sample groups, cell lines and a type of sample fraction used to generate RNA sequencing libraries are indicated. FIG. 1C) All mapped reads from cellular and exosomal fractions are grouped by annotation to cellular transcripts from which they originate and presented as distribution frequency of mapped reads in %.

FIGS. 2A-2D: MicroRNAs are non-randomly incorporated into exosomes. FIG. 2A) MiRNAs were classified in five groups indicated at the x-axis according to the ratio between the amount of miRNAs released from the cells and the amount retained in the cell after normalization (reads per million; RPM). The data is plotted as percentage of miRNAs fraction distributed over five groups. FIGS. 2B and 2C) Significant over-represented miRNAs in exosomes and cells are depicted at the x-axis with their read abundance exceeding 100 reads per million (normalized, RPM) at the y-axis (on logarithmic scale). The error bars are the SD of the mean (LCL 1-3 samples; n=3). FIG. 2D) MiRNA detection in LCL cells and their paired exosomes by stem-loop-based qRT-PCR. Data represents ΔΔCt value of technical duplicates normalized to miR-92a Ct value from two independent experiments.

FIGS. 3A and 3B: MicroRNAs are non-randomly incorporated into Burkitt's Lymphoma exosomes. MiRNAs from exosomal and cellular fractions of BLs (BL1 and 2) are analyzed as LCL samples. edgeR analysis yielded statistically significant over-represented miRNAs in (FIG. 3A) exosomes and (FIG. 3B) cells with their read abundance exceeding 100 reads per million (normalized, RPM). The y-axis represents the relative abundance of the non-randomly distributed miRNAs. MiRNAs that are significantly expelled or retained are compared with their retained or expelled equivalents. The error bars are the SD; (n=2); y-axis is in logarithmic scale.

FIGS. 4A and 4B: EBV-encoded miRNAs exhibit stronger tendency for cellular retention than human miRNAs. FIG. 4A) Human (LCL) and EBV miRNAs were classified in five groups according to the ratio between the amount of miRNAs released from the cells and the amount retained in the cell after normalization (RPM). The data is plotted as percentage of miRNAs fraction distributed over five groups. FIG. 4B) Human (LCL1) and EBV miRNAs were classified according to the Fold Change (defined as exo/cell log 2) between miRNAs released from the cells and miRNAs retained in the cell after normalization (RPM). Vertical lines indicate miRNA fractions with >four-fold increase in cell retention or exosome-associated release. Red lines indicate distribution of viral miRNAs among human miRNAs (gray).

FIGS. 5A-5C: Distribution of mature miRNAs and their isoforms in individual LCL samples (LCL 1, 2 and 3; cells vs. exosomes). FIG. 5A) Sequencing reads for all miRNAs detected in each library that mapped to annotated miRNA sequence are further dissected to mature (canonical) and isoform sequences. Those are further sub-divided into post-transcriptionally modified isoforms (non-templated nucleotide additions; NTAs) and post-transcriptionally unmodified isoforms (truncations and elongations). FIG. 5B) Samples are plotted against the percentage of the sum of all reads assigned to individual miRNAs per sample (for mature vs. isoforms) and FIG. 5C) against the sum of all miRNA reads with the NTAs.

FIG. 6: Statistical analysis and the significance of the effect of 3′-end NTA-type on miRNA variants distribution between cells and exosomes. False discovery rate-corrected p-values for 118 miRNAs expressed in all 12 samples. P-values derived from a logistic model comparing counts proportion of each NTA (A, U, C and G) between cell and exosome. In each graph, the circle color indicates the direction of the effect: exo>cell means that the data indicates larger proportions in exosomes compared to cells, and exo<cell means that the data indicates larger proportions in cells compared to exosomes. The results show that 3′ end adenylated miRNA isoforms are preferentially retained (chi-square test, p<0.05), while all other NTAs are preferentially released (3′ end-U: chi-square test, p=0.02; 3′ end-C: Fisher's exact test, p=0.04; and 3′ end-G: Fisher's exact test, p=0.02).

FIGS. 7A-7D: The 3′ end post-transcriptional modification defines retention or release of miRNA. FIGS. 7A and 7B) Distribution of mature miRNAs and their NTA-modified or unmodified isoforms in LCL samples was compared between cells and exosomes. The error bars are the SD of the mean (LCL 1-3 samples, n=3); * P-value=0.005 (Student's t-test). FIG. 7C) Distribution of miR-486-5p between individual LCL cells and exosomes libraries. All sequencing reads that mapped to miR-486-5p were summed and the contribution of (iso)form types of miR-486-5p is expressed as percentage. FIG. 7D) Detection of canonical miR-486-5p, 3′-end adenylated miR-486-5p (3′-AAA) and 3′ end uridylated miR-486-5p (3′-UUU) in cellular (LCL) and exosomal RNA fractions by qPCR. Data represents mean cycle threshold (Ct) value of technical duplicates from two independent experiments (error bars, SD).

FIGS. 8A-8F: The extent of 3′ end adenylation increases retention while 3′ end uridylation demarcates exosomal small RNA cargo. FIGS. 8A and 8D) Frequency plots of a pair-wise analysis of 3′-A and 3′-U isoforms. MiRNAs found to be expressed in both cells and exosomes were selected (at >300 reads, 100 miRNAs) for distribution analysis of their adenylated and uridylated reads expressed as percentage to the total of all NTA-modified reads. Red circles: isoforms with higher percentage in exosomes; Purple circles: isoforms with higher percentage in cells; White circles: equal distribution. FIGS. 8B and 8C) Fraction of human miRNAs and viral miRNAs for which the adenylated reads (3′ end-As) show retention or release tendency measured against the non-adenylated reads of the same miRNA. Adenylated miRNAs have a higher probability to be retained and it increases with the number of added adenines. FIGS. 8E and 8F) 3′-end uridylation rather than adenylation is more frequent on miRNA isoforms present in LCLs exosomes, BLs exosomes and in human urine exosomes. The bars represent the weighted mean of isoform reads per sample (exosomes only) with 3′ end NTA; nucleotide type indicated on the x-axis. The error bars are the SD of three samples for LCLs (n=3); p=0.005; two samples for BLs (n=2); p=0.001; and six samples from human urine (n=6); p<0.0001 (Student's t-test).

FIGS. 9A and 9B: 3′-end post-transcriptional modification affects distribution of processed small ncRNAs. FIG. 9A) Y RNA fragments with 3′ end adenylation or FIG. 9B) 3′-end uridylation (right panel) are differentially distributed between cellular and exosomal fractions. RNA fractions that correspond to processed Y RNA fragments that are derived from RNY1, RNY3, RNY4 and RNY5 (x-axis) are plotted against the percentage of RNA fragments with 3′ end modifications.

FIG. 10: List of mapped reads identified in urine exosomes by small RNA sequencing. Summary of sample characteristics and RNA-seq data from human urine extracellular vesicles. cDNA libraries were generated from exosomal small RNA fractions after purifying urine exosomes. Total number of reads from all libraries before and after mapping to genome of interest (hsg 19/GRCh37, patch 5) is specified. To provide annotations to RNA elements that mapped to human genome, all mapped reads were analyzed against currently known databases: 1) mature and pre-miRNA sequences from miRBase version 19, 2) NCBI Reference Sequences human RefSeq genes downloaded May 2013, 3) tRNA sequences from the genomic tRNA database, 4) repeat-derived sequences detected by means of the RepeatMasker algorithm downloaded from UCSC and 5) piwiRNA (piRNAs) downloaded from the NCBI nucleotide database.

FIGS. 11A-11C: Healthy renal tissue biopsies vs. Clear cell Renal Cell Carcinoma (kidney tissue) biopsies. FIG. 11A) MicroRNA diversification. Sequencing reads detected in each library that mapped to annotated miRNA sequence are grouped and presented as one miRNA. Those mapped reads consist of mature (canonical) and isoform sequences. Isoforms are further sub-divided into enzymatically modified isoforms (3′-end NTA non-templated nucleotide addition) and unmodified isoforms (truncations and elongations occurring at both 3′-end and 5′-end. FIG. 11B) The analysis of publicly available data from clear cell renal cell carcinoma (CCRC; four experimental groups) by RNASeq. Data analysis based on mature (miRBase) sequences shows poor distinction between experimental groups for selected miRNAs. FIG. 11C) The ratio between isomiRs and mature miRNAs in all four experimental groups revealed pronounced differences for 3′-end adenine additions (adenylation), especially between the clinically relevant groups. Significant differences in proportion of adenylated miRNAs permit separation between clinically defined groups.

FIGS. 12A-12C: Post-transcriptional modifications stratifying healthy versus prostate cancer patients. Although the table states that the data is presented as “C/V,” the ratios are in fact presented as “V/C.” This change does not affect the STD or p-values.

FIGS. 13A and 13B: Non-invasive strategies for monitoring vital tumor tissue in malignant Lymphoma patients. FIG. 13A) Left: FDG/PET image of a classical Hodgkin's Lymphoma patient before treatment. The black arrow indicates multiple metabolically active tumor masses. Right: fused PET/CT image of the same patient. White arrow indicates a vital tumor mass. FIG. 13B) Lymphoma tumor cells (upper dark brown) and normal cells (lower light brown) actively secrete 100 nanometer vesicles, including MVB-derived exosomes into circulation. These extracellular vesicles (EVs) contain miRNAs that are protected against external RNAses. Besides being encapsulated by EVs, miRNAs can be associated with and protected for degradation by proteins and HDL, however, these do not reflect vital tumor cells. Moreover, dying cells release biomolecules (i.e., RNA, DNA, protein) into circulation. The figure is adapted from Hori et al., 2013, Science Transl. Med.

FIGS. 14A-14E: Single step size-exclusion chromatography (SEC) separates circulating extracellular vesicles (EV) from protein/HDL for optimal miRNA detection in patient plasma. FIG. 14A) qEV size exclusion chromatography (SEC) column (recently commercially available from IZONtm). The qEV columns allow single-step reproducible isolation of circulating EVs from 1-1.5 ml plasma, separating them from circulating protein/HDL (Boing et al., JEV 2014). FIG. 14B) EM image of plasma EVs isolated from a cHL patient using sepharose CL-2B SEC. The size bar indicates 200 nm and most EVs are in the range of 100 nm although larger (200 nm) vesicles are present. FIG. 14C) EM image of the protein/HDL fraction of the same patient plasma as in FIG. 14B. Particles that resemble EVs are not observed. FIG. 14D) Particle analysis using qNano (IZONtm), based on Tunable Resistive Pulse Sensing (TRPS). cHL patient plasma EV and protein/HDL fractions are separated using the qEV device. The EV fraction (orange) is highly enriched in particles with a size-distribution that corresponds to the EM image in FIG. 14B. FIG. 14E) RT-PCR analysis of EV and protein/HDL fractions separated by SEC. The EV fractions 9-12 are highly enriched for vtRNA1-1, while protein/HDL fractions (19-22) are enriched for miR92a consistent with (Arroyo et al., PNAS 2011).

FIGS. 15A-15D: Selection and validation of candidate miRNA biomarkers in plasma EV. FIG. 15A) Hierarchical clustering of miRNAs from vesicles. RNAseq analysis of Lymphoma cell line-derived exosomes (n=7) and plasma extracellular vesicles from a cHL patient (before treatment) and a healthy control shows clearly distinct profiles. FIG. 15B) List of the ten most abundant miRNAs in Platelets, PBMCs and EVs (Ple et al., PLoSone 2012). FIG. 15C) Comparison of the level of miRNAs in plasma EV and Hodgkin's cell line-derived exosomes (L1236) yields potential cHL stroma-derived and tumor-associated miRNA markers. MiRNAs shown differ by log(FC)>1. FIG. 15D) Boxplots showing RT-PCR data from four candidate miRNA biomarkers in plasma EVs isolated from cHL patients (n=10) and healthy controls (n=4). miR1973 is abundantly detected in plasma EV from both cHL patients and healthy controls. P values were calculated using a two-tailed student's t-test.

FIGS. 16A-16F: miR-21-5p and let7a-5p levels in plasma EV decrease during successful treatment. FIG. 16A) PET images from a cHL patient before (left) and after (right) two cycles of first line treatment (BEACOPP). Arrow indicates metabolically active tumor. FIG. 16B) RT-PCR analysis shows a strong decrease (70%) of EV-associated miR-21-5p and let-7a-5p during treatment. MiR-21-5p and let-7a-5p levels are normalized to miR-1973 and shown as fold decrease (error bars, SEM). FIGS. 16C and 16D) MiR21-5p and let7a-5p levels in EVs decrease in both de novo patients during BEACOPP treatment (FIG. 16C) and in relapse patients after treatment with DHAP/ADC followed by autologous stem cell transplantation (FIG. 16D) as determined by RT-PCR. MiR-21-5p and let-7a-5p levels are normalized to miR-1973 and shown as fold decrease (error bars, SEM). FIGS. 16E and 16F) MiR-21-5p and let7a-5p levels in EVs decrease in cHL patients responding to therapy as determined by decreasing serum TARC levels (FIG. 16E) but not in cHL patients where TARC levels remain high (FIG. 16F). MiR21-5p and let7a-5p levels are normalized to miR1973 and shown as fold change (error bars, SEM).

FIGS. 17A and 17B: Candidate miRNA biomarker levels in plasma EV remain low during follow up. FIG. 17A) MiR-127-3p, miR-155-5p, miR-21-5p and let-7a-5p levels in cHL patient plasma EV are decreased during (t=1 month) and after (t=3 months) BEACOPP treatment and remain low up to 6 months after therapy. MiRNA levels are normalized to miR-1973 and shown as fold decrease (error bars, SEM). FIG. 17B) RT-PCR analysis shows that miR-21-5p and let-7a-5p levels in plasma EV of a healthy donor are stable. Data is shown as Ct values (error bars, SEM).

FIG. 18: Increased levels of candidate miRNAs in cHL plasma EV are disease-specific MiR-127-3p, miR-155-5p, miR-21-5p and let-7a-5p; RT-PCR levels are increased in cHL patient plasma EV (n=10), but not in autoimmune (SLE) patients (n=3) compared to healthy controls (n=4). Data is shown as Ct values (error bars, SEM). P values are calculated using a two-tailed student's t-test.

FIGS. 19A-19C: NTA analysis on miRNAs in plasma EVs of healthy and Hodgkin's patients. Sequencing was performed on multiple plasmas from healthy donors and cHL patients in both protein and vesicle fractions and analyzed the data with sRNA bench (http://arn.ugr.es/srnabench/). FIG. 19A) Graph showing the proportion of all identified miRNAs with the 3′-end NTA defined as A or U. Only the cHL EV fraction has a higher proportion of the 3′-end uridylated miRNAs. FIG. 19B) Same as in A but now the data represents differences for 3′-end post-transcriptional NTA-based modification of miR-486-5p. FIG. 19C) TAQMAN® RT-PCR as previously described (Koppers-Lalic et al., 2014) for miR486-5p in various fractions of two donors confirming RNAseq data as shown in FIG. 19B.

FIG. 20: Graphical representation of mapped reads that align to miR-92a genomic sequence (SEQ ID NOS: 198-209). Post-transcriptional modifications (NTA) and elongations/truncations of 5′ or 3′-end nucleotides are indicated with underlined letters (for elongation) or with * for truncations.

FIGS. 21A-21S: Exemplary biomarkers. Table 2 depicts tRNA biomarker pairs from urine in prostate cancer (PCa) patients. Table 3 depicts miRNAs that characterize cHL. Tables 4 and 10 depict ncRNA biomarker pairs in extracellular vesicles extracted from blood plasma of healthy donors and cHL patients. Tables 5 and 11 depict ncRNA biomarker pairs in protein fraction (PF) extracted from blood plasma of healthy donors and cHL patients. Table 6 depicts ncRNA biomarker pairs in blood serum of breast cancer patients. Table 7 depicts ncRNA biomarker pairs in testicular germ cell tumors. Table 8 depicts ncRNA biomarker pairs in colorectal cancer. Table 9 depicts ncRNA biomarker pairs in blood serum of Alzheimer's patients.

### DETAILED DESCRIPTION

The articles “a” and “an” are used herein to refer to one or to more than one (i.e., to at least one) of the grammatical object of the article. By way of example, “an element” means one element or more than one element.

As used herein, “bodily fluid” refers to a bodily fluid comprising ncRNA including blood (or a fraction of blood such as plasma or serum), lymph, mucus, tears, saliva, sputum, urine, semen, stool, CSF (cerebrospinal fluid), breast milk, and ascites fluid. Preferably, the bodily fluid is urine. Preferably, the bodily fluid is selected from blood. As used herein, “blood” also includes blood serum and blood plasma. Preferably, the bodily fluid is blood plasma.

“Biomarker” may be used to refer to a biological molecule present in an individual at varying concentrations useful in predicting the health status of an individual.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, the additional component(s) not altering the unique characteristic of the disclosure.

As used herein, “health status” refers to the overall (physical/physiological) condition of an individual at a particular time. Health status includes the presence or absence of disease or disorders, e.g., neurological disorders (such as Alzheimer's disease), cancer/tumors, infectious disease, metabolic diseases (e.g., amyloidosis), cardiovascular diseases, and immunological disorders. Preferably, the disorder is selected from prostate cancer, breast cancer, cervical cancer, lymphoma, colon cancer, glioblastoma and lung cancer.

Health status also includes the risk of developing such disorders (i.e., having a decreased or increased risk over the general population or individuals of similar age, genetic background, environmental risk factors, etc.). Health status also includes the particular stage of disease/disorder as well as the severity and prognosis (e.g., survival prognosis). Health status also refers to the prognosis of an individual to be effectively treated with a particular agent. “Classifying a health status” includes classifying an individual as healthy; as having or not having a particular disorder; as having an increased, decreased, or normal risk of developing a disorder; as being a good or poor responder to a particular treatment; as having a particular stage or severity of a disease; as having a good or poor survival or recovery prognosis.

As used herein, an “individual” refers to humans and animals, e.g., mammals such as a domestic animal (e.g., dog, cat), a farm animal (e.g., cow, sheep, pig, horse) or a laboratory animal (e.g., monkey, rat, mouse, rabbit, guinea pig). Preferably the individual is a human.

As used herein, “small non-coding RNA” (ncRNA) refers to RNA that is not translated into protein and includes transfer RNA (tRNA), ribosomal RNA (rRNA), snoRNAs, microRNA (miRNA), siRNAs, small nuclear RNA (snRNA), Y RNA, vault RNA, antisense RNA, tiRNA (transcription initiation RNA), TS Sa-RNA (transcriptional start-site associated RNA) and piwiRNA (piRNA). Small ncRNA have a length of less than 200 nucleotides.

Preferably, ncRNA is a small Pol III RNA, preferably selected from tRNA, miRNA, snRNA, Y RNA, vault RNA, and snRNA. Preferably, ncRNA is selected from miRNA, Y RNA, vault RNA, and more preferably, ncRNA is miRNA.

Preferably, a small ncRNA as used herein is between 16 and 200 nucleotides, more preferably between 16 and 100 nucleotides, even more preferably between 16 and 40 nucleotides. An ncRNA may be of endogenous origin (e.g., a human miRNA) or exogenous origin (e.g., virus, bacteria, or parasite).

“Canonical” ncRNA refers to the sequence of the RNA as predicted from the genome sequence and is the most abundant sequence identified for a particular RNA. For miRNA, this refers to miRNAs formed via the “canonical miRNA pathway.” Precursor miRNA (pre-miRNA) is cleaved into a short hairpin RNA and is then exported into the cytoplasm for processing by a Dicer enzyme. The resulting “canonical” mature miRNAs are usually 21-22 nucleotides in length.

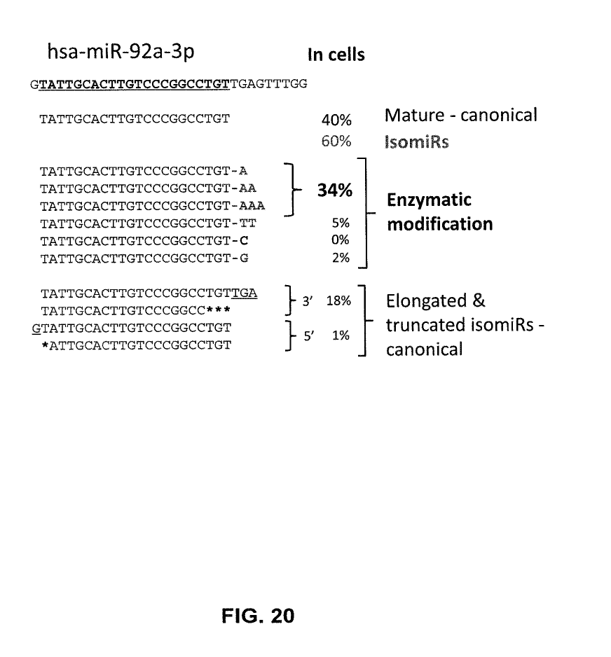

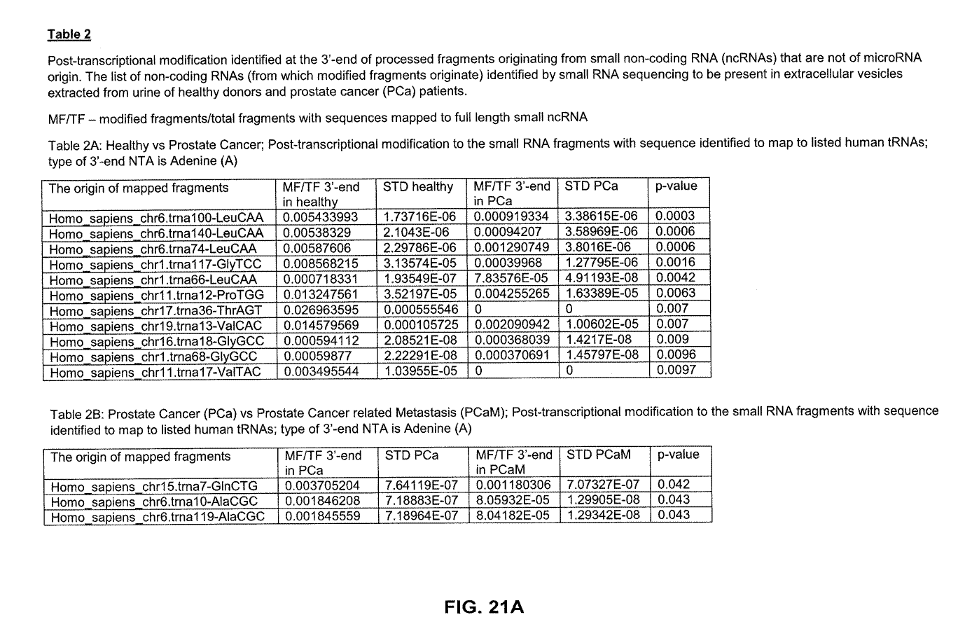

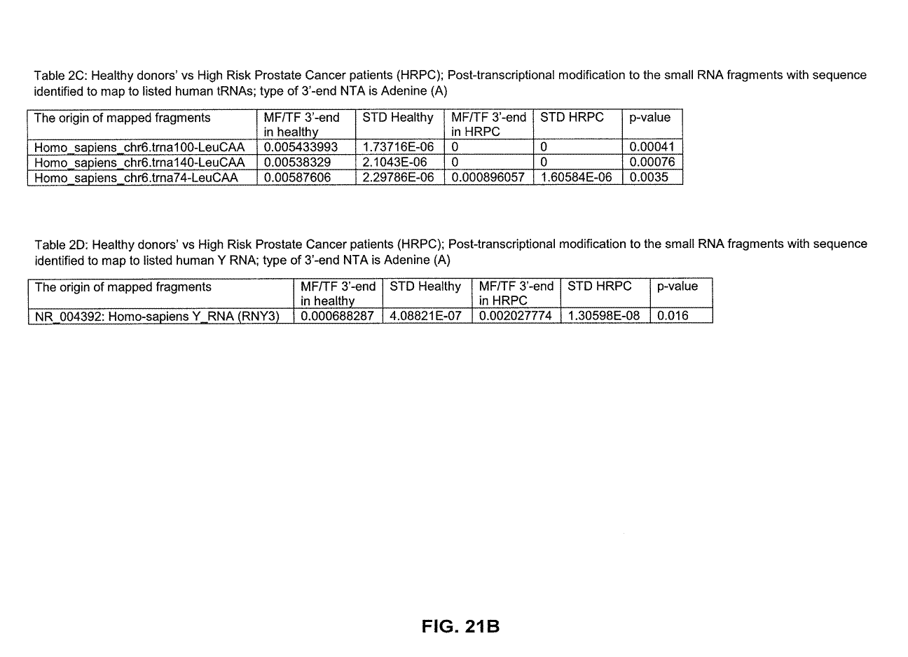

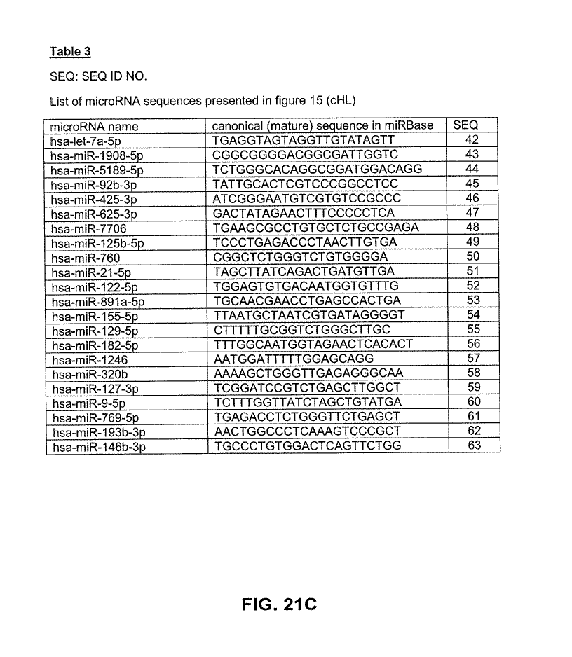

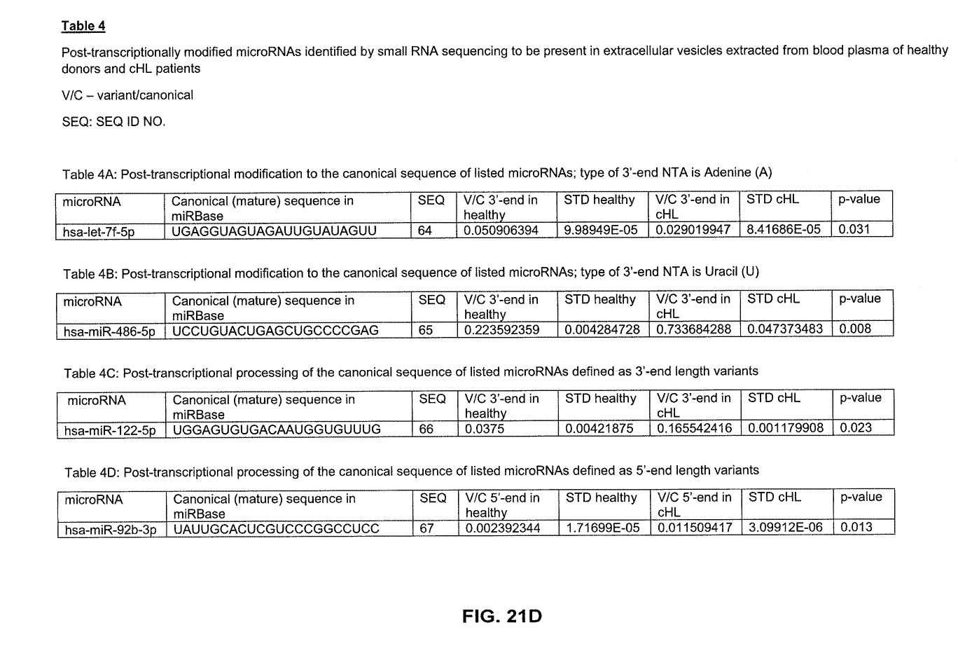

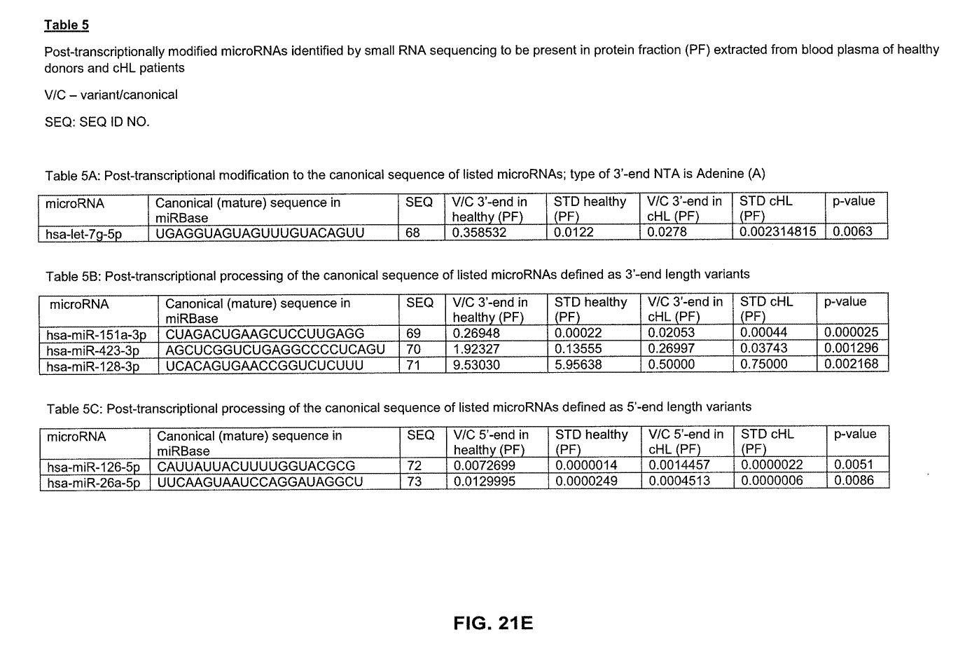

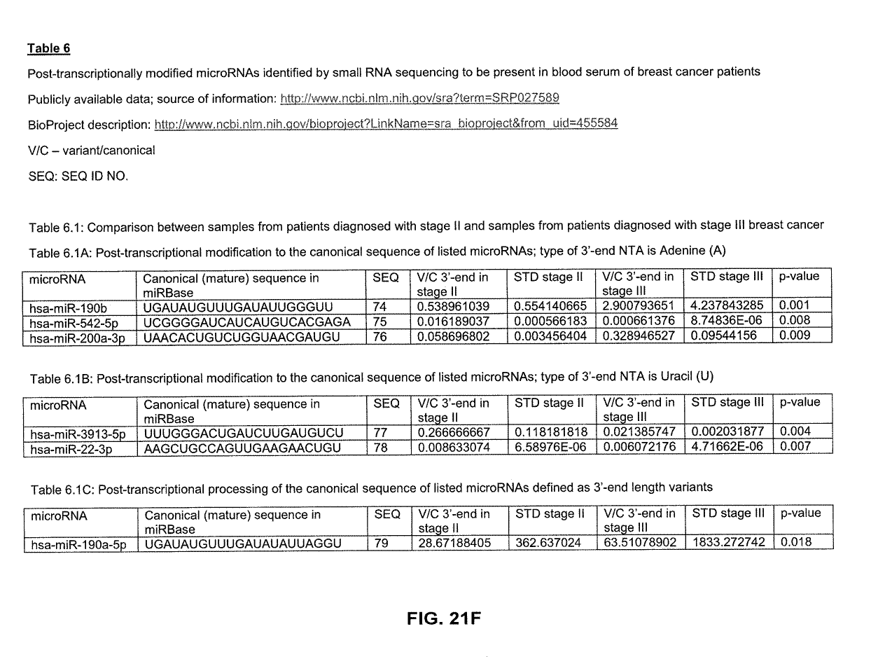

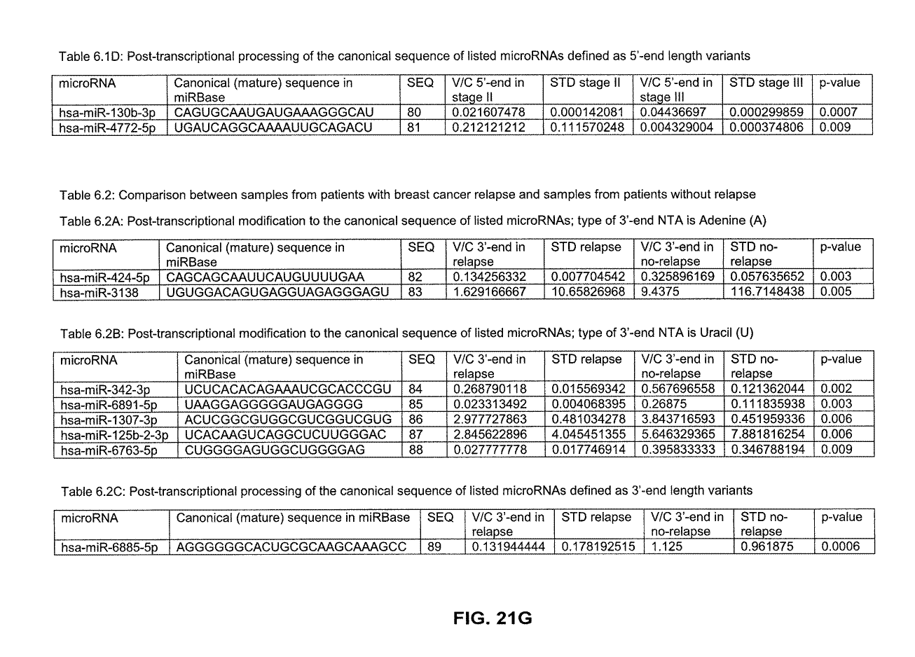

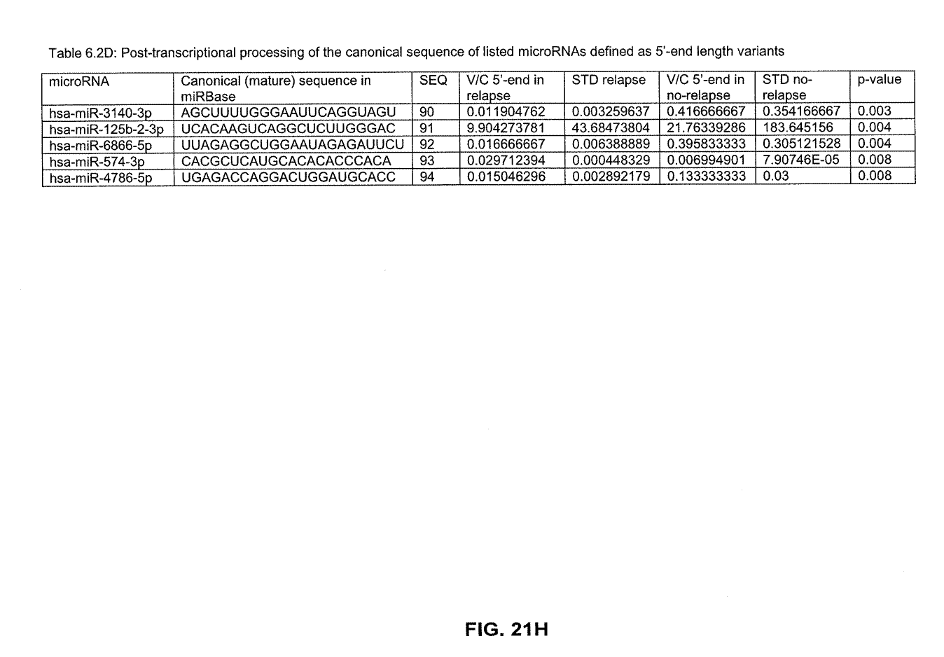

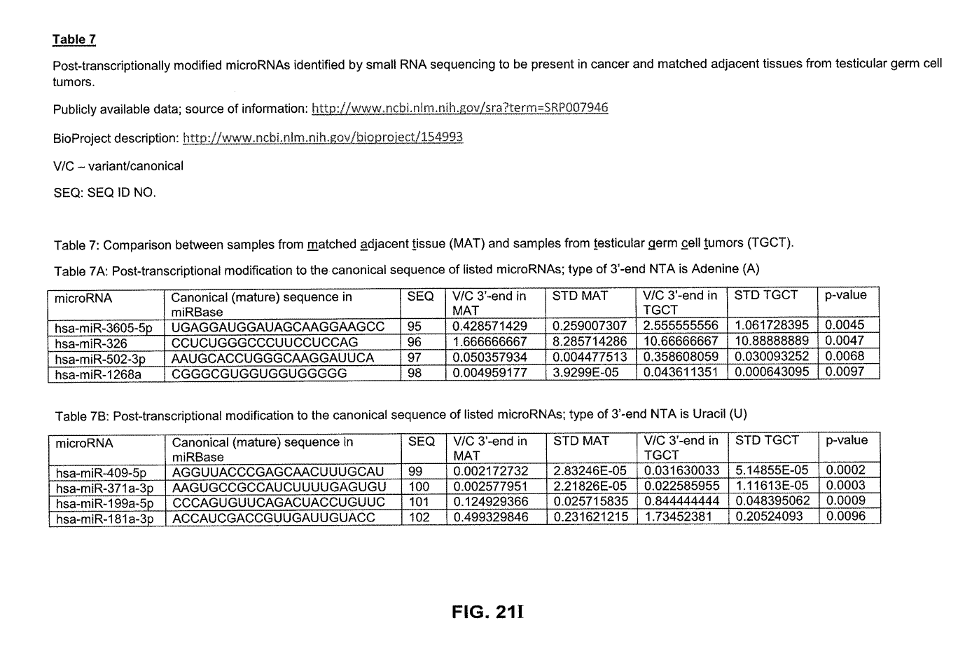

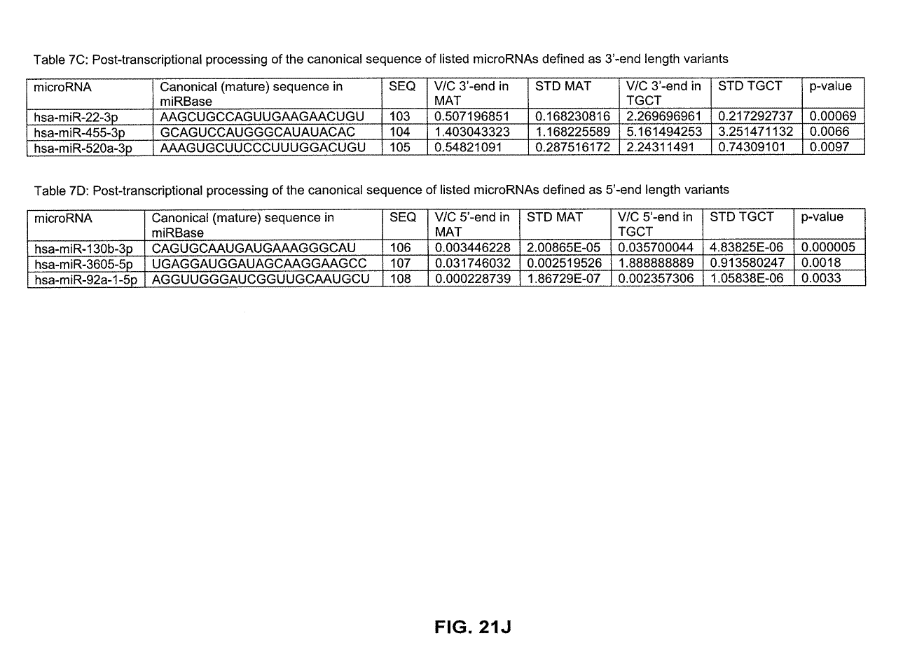

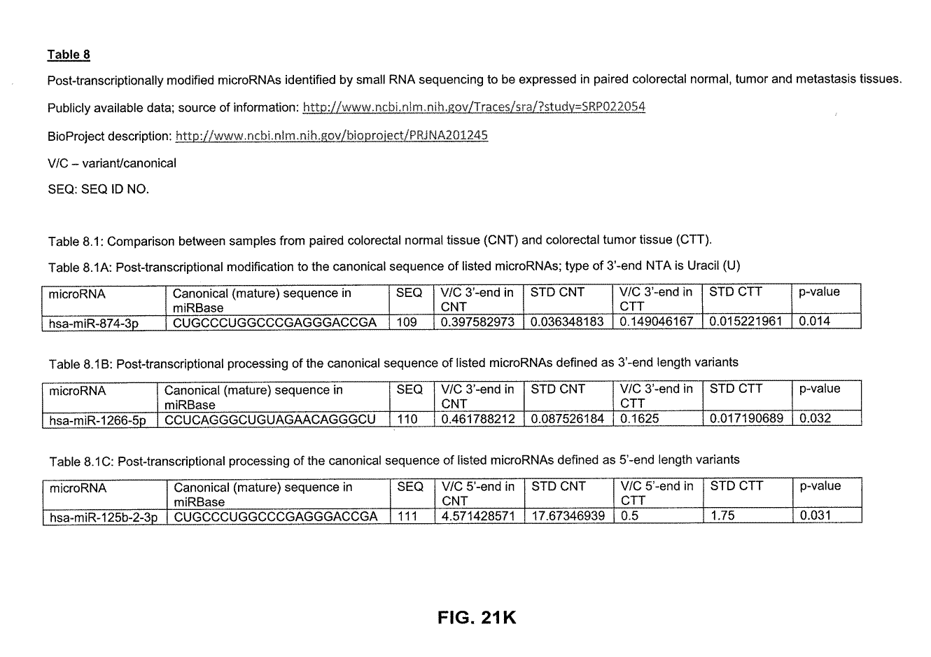

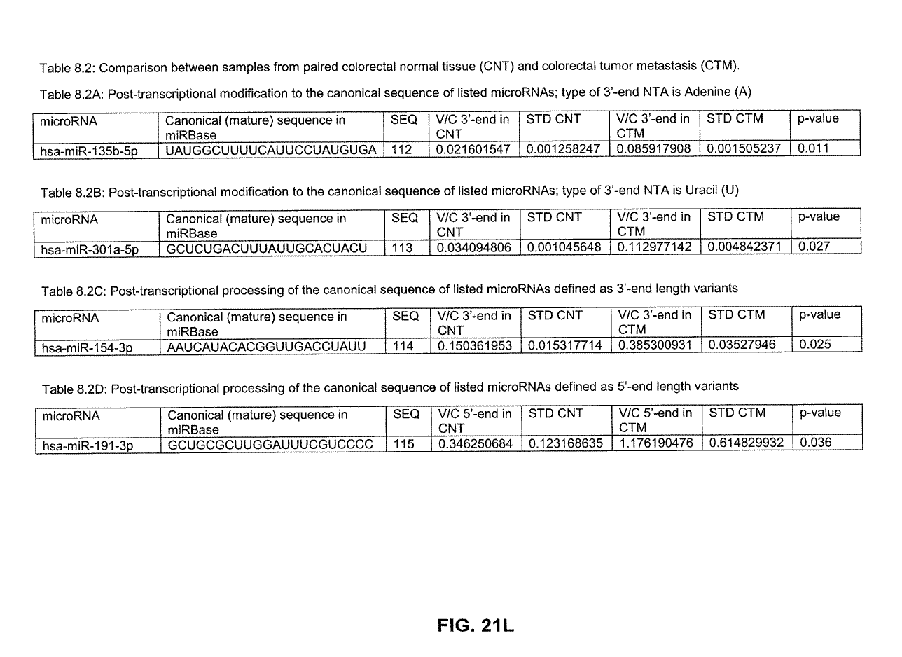

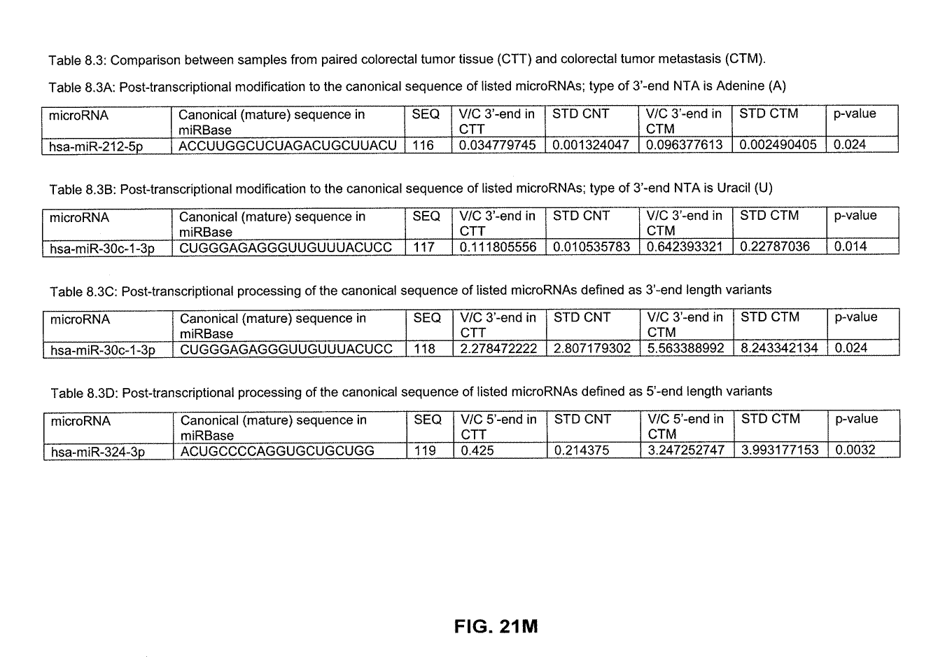

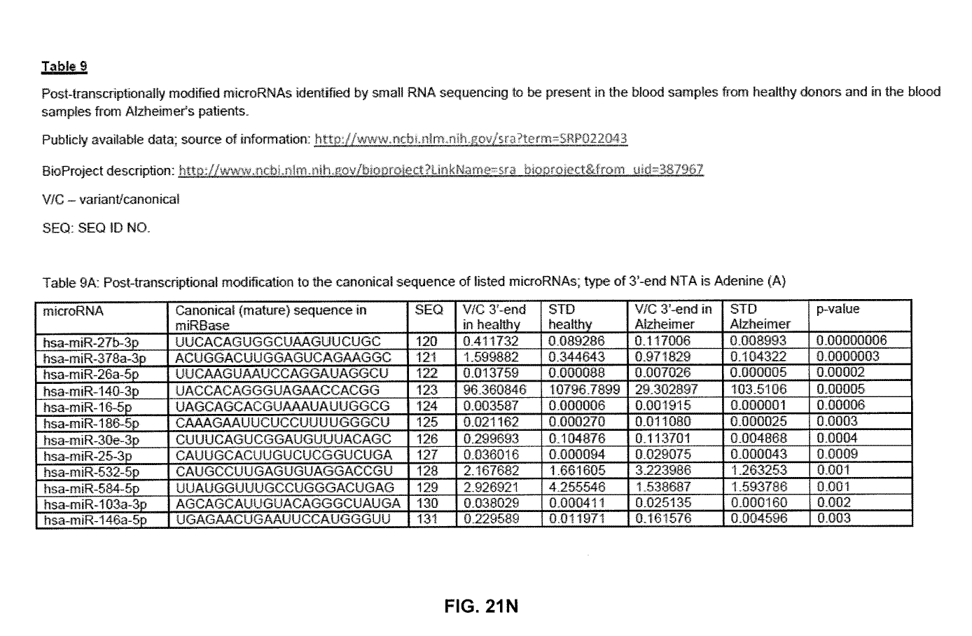

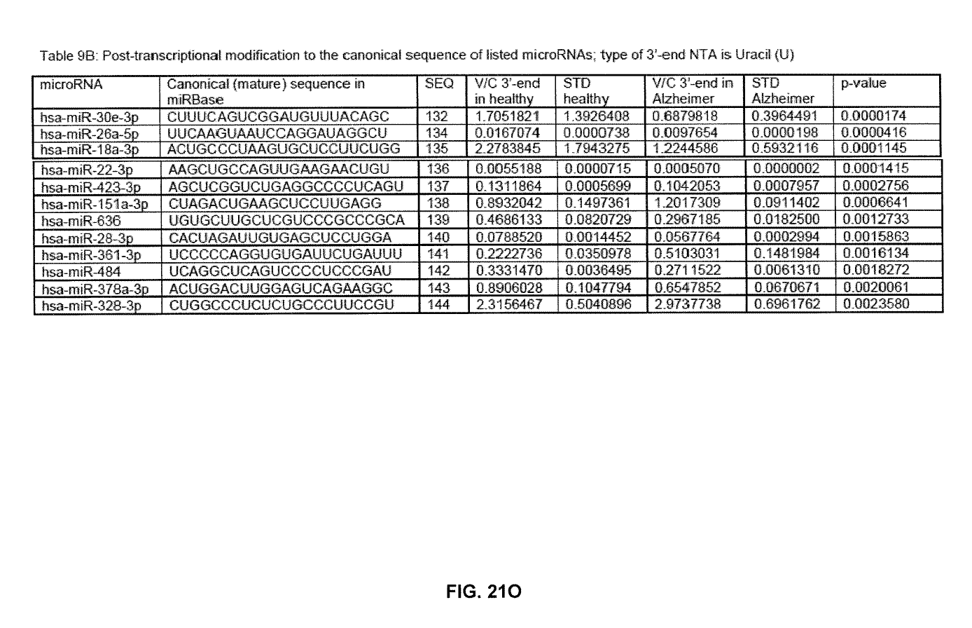

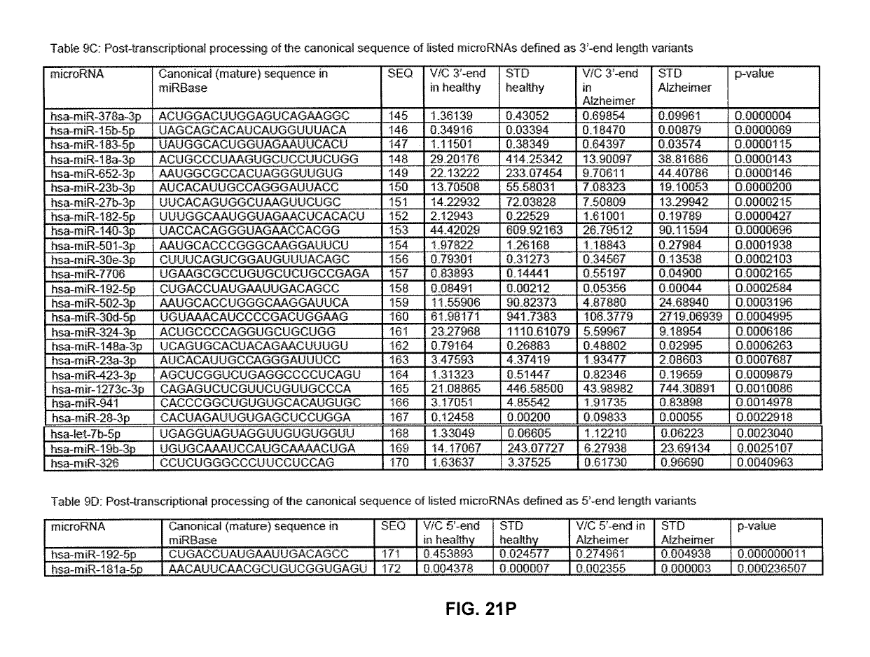

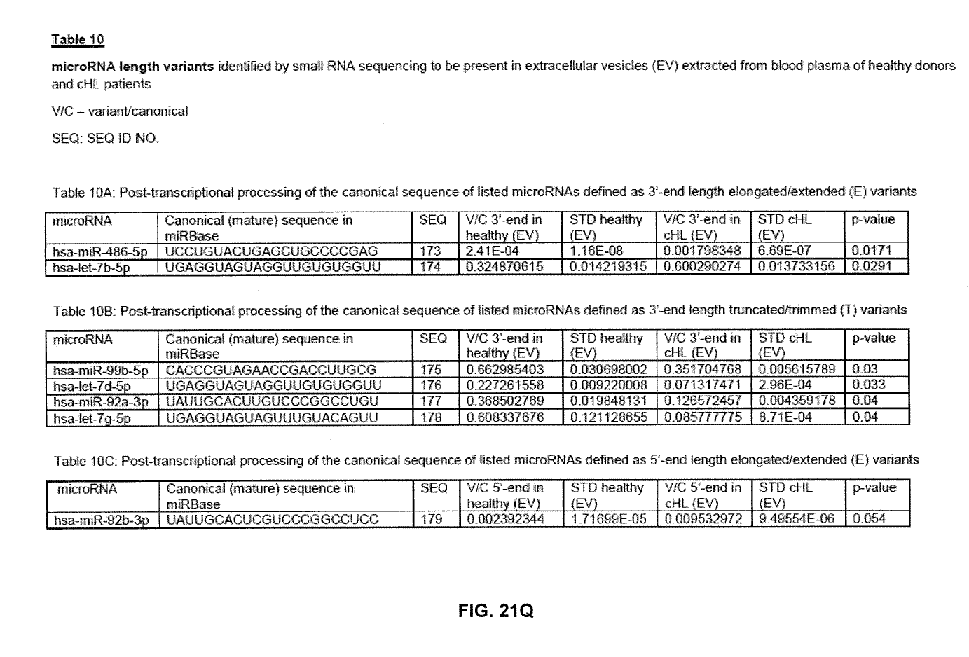

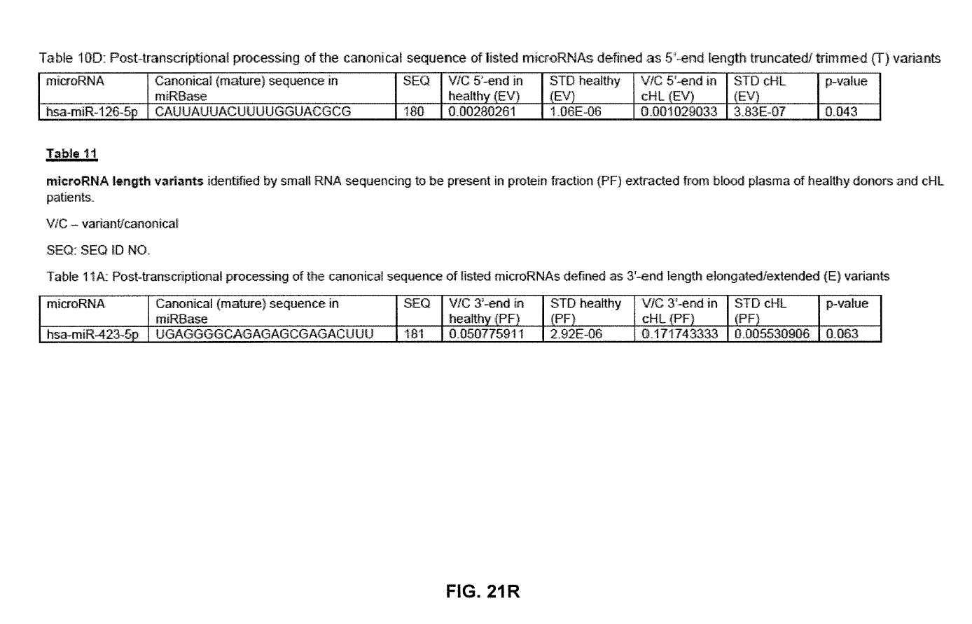

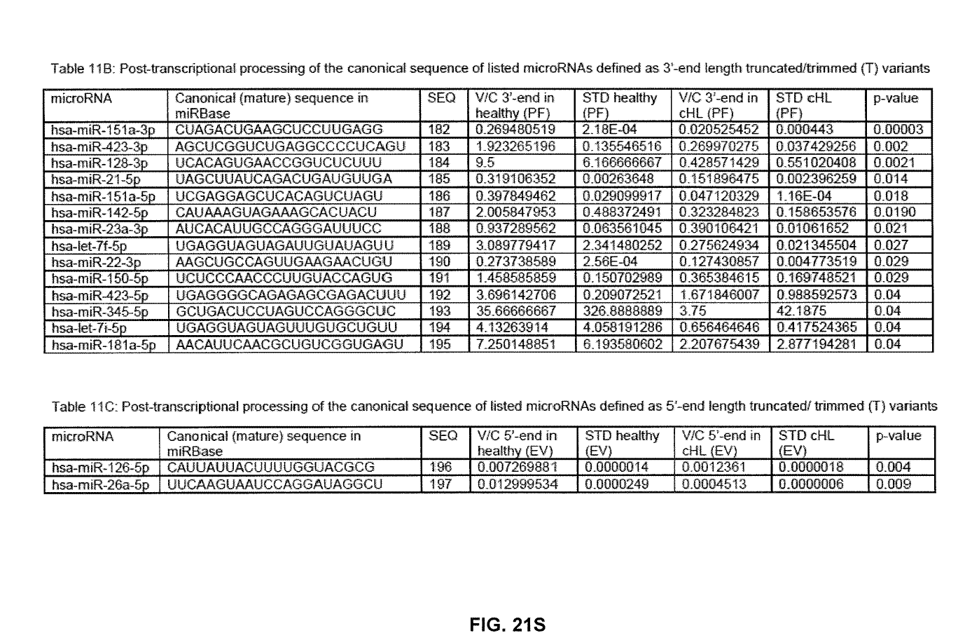

“Trimmed” ncRNA refers to an ncRNA in which exonuclease-mediated nucleotide trimming has removed one or more nucleotides at the 5′ and/or 3′ end of the molecule. Preferably, the trimming is a 3′ trimming. Preferably, one nucleotide is trimmed from the 3′ end of a canonical sequence. Trimmed miRNA can be easily detected since the start and stop sites of canonical miRNAs are known. Examples of trimmed ncRNAs are depicted in FIG. 20 and FIGS. 21A-21S (see, e.g., Table 11C).

3′ non-templated nucleotide addition (3′ NTA) refers to post-translational additions of one or more nucleotides to the 3′ end of an RNA, usually by RNA nucleotidyl transferases, such as PAPD4, PAPD5, ZCCHC6, and ZCCHC11 (Burroughs et al., 2010; Polikepahad and Corry, 2013). The most common forms are adenylation (3′ NTA-A) and uridylation (3′ NTA-U), but the addition of cytosine (3′ NTA-C) and guanine (3′ NTA-G) are also possible. Although generally one or two nucleotides are added, the addition of three or more nucleotides is also possible. Before NTA occurs, the canonical sequence is optionally trimmed at the 5′ or 3′ end. Preferably, the 3′ NTA is a single nucleotide addition selected from 3′ NTA-A, 3′ NTA-G, 3′ NTA-U, and 3′ NTA-C. Examples of 3′ NTA ncRNAs are depicted in FIG. 20 and FIGS. 21A-21S (see, e.g., Table 2A).

“Extended ncRNA” refers to an miRNA that is longer than the canonical miRNA sequence and is a term recognized in the art. The nucleotides making up the extension correspond to nucleotides of the precursor sequence and are, therefore, encoded by the genome in contrast to non-templated nucleotide addition. While not wishing to be bound by theory, it is thought that extended ncRNAs are the result of differential precursor miRNA processing. In general, such extensions may comprise 1-5, usually 1-3, extra nucleotides as compared to the canonical miRNA sequence. Extended miRNA can be easily detected since the start and stop sites of canonical miRNAs are known. Examples of extended ncRNAs are depicted in FIG. 20 and FIGS. 21A-21S (see, e.g., Table 11A).

The ncRNA variants are selected from the canonical sequence of the ncRNA, a 5′ or 3′ trimmed version of the ncRNA, a 5′ or 3′ extended version of the ncRNA, a 5′ or 3′ trimmed version of the ncRNA, or 5′ or 3′ extended version of the ncRNA (collectively referred to herein as “length variants”) and the ncRNA having a 3′ non-templated nucleotide addition (3′ NTA). As used herein, “ncRNA variants” refers to a group of sequences that originate from a single ncRNA gene. Each variant differs in sequence from each other, e.g., by 5′ or 3′ trimming or by the presence or absence of 3′ NTAs. The variants may have the same or different biological function.

While not wishing to be bound by theory, it is believed that trimmed ncRNA (i.e., truncated) and extended ncRNA (i.e., elongated) may result for similar inefficiencies in pre-miRNA processing. In some embodiments, the ncRNA variant is a “length variant,” i.e., a trimmed or extended ncRNA. For example, Table 5B lists biomarker pairs where one variant is the canonical structure and the second variant is a 3′ length variant. In some embodiments, it is preferred to separate the length variants into ncRNA extensions and trimmed ncRNAs. Table 11 is an analysis of the same data, but where the length variants are split into extensions (see, Table 11A for 3′ end extensions) and trimmed ncRNAs (see, Table 11B for 3′ end trimmed variants).

Quantify and quantification may be used interchangeably, and refer to a process of determining the quantity or abundance of a substance in a sample (e.g., a biomarker), whether relative or absolute. For example, quantification may be determined by methods including but not limited to, micro-array analysis, qRT-PCR, band intensity on a Northern blot, targeting small RNA sequencing or by various other methods known in the art.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

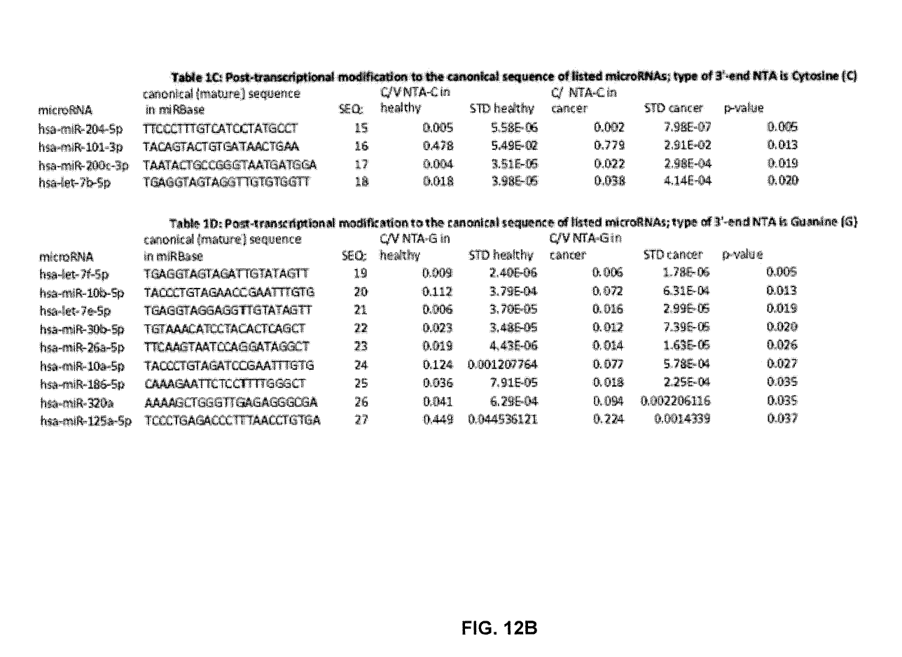

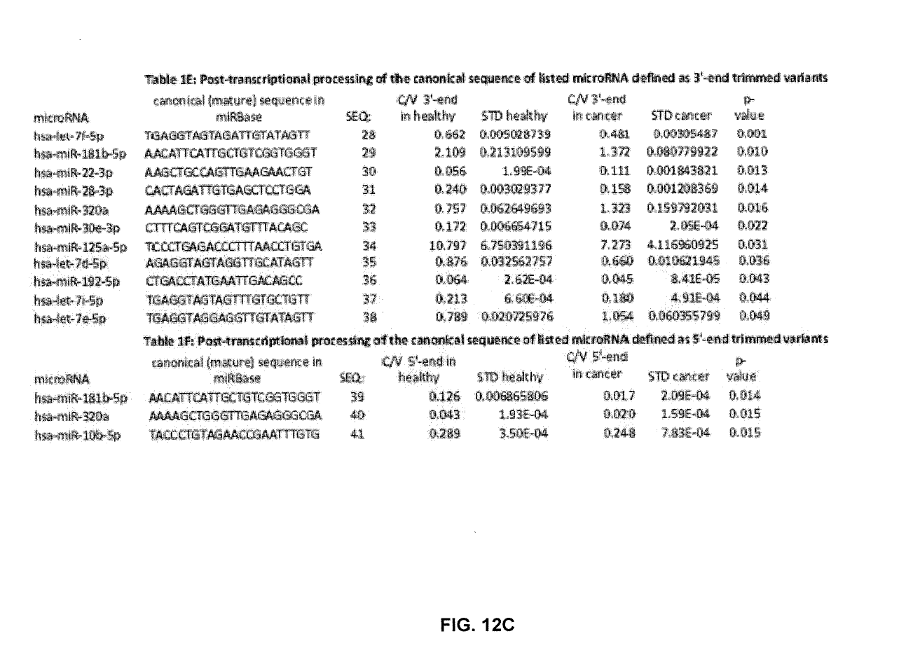

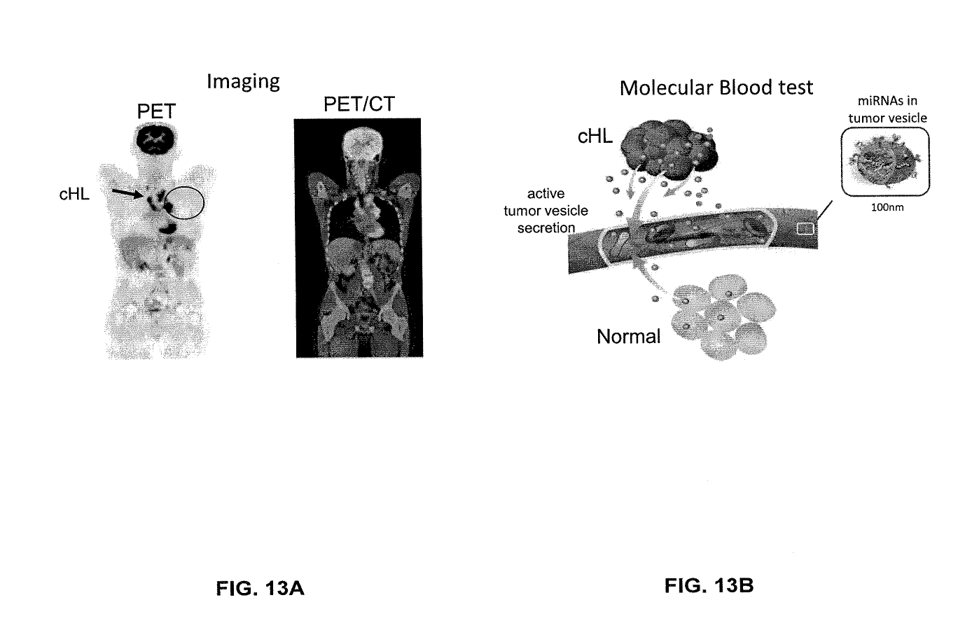

In one aspect, the disclosure provides a method for identifying a small non-coding RNA (ncRNA) biomarker pair. Preferably, the ncRNA biomarkers are of at least 16 nucleotides in length. Example 3 describes biomarker pairs for discriminating healthy patients from those having prostate cancer, which were identified using a method as described herein. The top biomarker pairs are disclosed in FIGS. 12A-12C.

The method comprises determining for an ncRNA, the ratio of two different varieties of the ncRNA in a first bodily fluid sample obtained from a first individual reference and in a second bodily fluid sample obtained from a second individual reference, wherein, the first individual reference has an altered health status from the second individual reference.

The bodily fluid samples are from one or more individuals or may be the average ratio in a population of individuals. The first individual reference (which may be one more individuals, preferably one) has an altered health status from the second individual reference (which may be one more individuals, preferably one). In some embodiments, the first and second individual references are from the same individual, wherein the health status of the individual has changed, e.g., the samples are obtained from an individual before and after a treatment regime.

Methods of identifying diagnostic and prognostic biomarkers are well-known in the art. The selection of the samples is well within the purview of one skilled in the art and can vary depending on the type of biomarker to be identified.

In the case of biomarkers of disease, an exemplary embodiment provides that the first individual reference is from an individual(s) having a disorder and the second sample from an individual reference(s) not having the disorder. Preferably, the disorder is cancer; more preferably, selected from cervical cancer, lymphoma, colon cancer, glioblastoma and lung cancer or prostate cancer.

In the case of biomarkers of risk, an exemplary embodiment provides the first individual reference from an individual(s) having an increased risk of developing a disorder and the second individual reference from an individual(s) not having an increased risk. For example, the samples could be provided from heavy-smoking individuals before the onset of lung cancer. The ratios are analyzed and after a certain amount of time, for example, 1 or 2 years, the status of lung cancer in the individuals is monitored.

In the case of prognosis biomarkers, an exemplary embodiment provides the first individual reference from an individual successfully treated and a second sample from an individual not successfully treated.

The ratio of RNA variety may be determined by methods known to a skilled person.

Preferably, the ratio is determined by quantifying the two different varieties in each sample and determining the relationship between the two quantities. This is preferably expressed as the quotient of one divided by the other. Preferably, the abundance of each variety is determined. More preferably, the relative abundance of each variety is determined.

In an exemplary embodiment, the ratio is (abundance of an ncRNA variety 1 divided by abundance of the canonical ncRNA) divided by (abundance of an ncRNA variety 2 divided by abundance of the canonical ncRNA).

Methods for quantitating RNA levels are well known. Several methods exist to add additional sequence to an ncRNA to facilitate priming and detection. In particular, a common sequence can be added to every ncRNA to allow for use of a single universal extension primer. In particular, QPCR-based ncRNA detection methods add additional sequence to the miRNA to increase its length prior to or during reverse transcription. There are also a number of commercially available systems for performing PCR assays on small RNAs (e.g., Applied Biosystems Custom TAQMAN® Small RNA Assays and miRCURY LNA™ microRNA PCR System from Exiqon). Preferably, the method for quantitating RNA levels is “deep sequencing.” This refers to methods that provide both the sequence and the frequency of an RNA molecule (see, e.g., US2012/0322691 for general methods and specific adaptor modifications).

The methods further comprise comparing the ratios from the first and second bodily fluid samples, and identifying the two different varieties of ncRNA as a biomarker pair when the ratio is altered between the first and second bodily fluid samples. An altered ratio between the first sample and second sample identifies ncRNA as an ncRNA biomarker. More precisely, the two ncRNA varieties are identified collectively as a biomarker. An altered ratio is a statistically significant difference between the ratios in the two samples. Preferably, an altered ratio is indicated when the ratio from the first sample falls outside of about 1.0 standard deviations, about 1.5 standard deviations, about 2.0 standard deviations, or about 2.5 stand deviations of the second sample.

The ncRNA varieties are selected from the canonical sequence of the ncRNA, a 5′ or 3′ trimmed version of the ncRNA, a 5′ or 3′ extended version of the ncRNA, a 5′ or 3′ trimmed version of the ncRNA or a 5′ or 3′ extended version of the ncRNA (i.e., “length variant”) and the ncRNA having a 3′ non-templated nucleotide addition (3′ NTA). Preferably, the first variety is selected from the canonical ncRNA and the second variety is the ncRNA with a 3′ non-templated nucleotide addition, and when ncRNA is miRNA, the first variety is selected from canonical ncRNA, the ncRNA trimmed at the 5′ or 3′ end, and the ncRNA with a 3′ non-templated nucleotide addition, optionally trimmed at the 5′ or 3′ end and the second variety is selected from ncRNA trimmed at the 5′ or 3′ end and ncRNA with a 3′ non-templated nucleotide addition, optionally trimmed at the 5′ or 3′ end. It is clear that the ratio is for two different varieties of the same ncRNA.

In exemplary embodiments, the ratio is selected from canonical:3′ NTA-A; canonical:3′ NTA-G; canonical:3′ NTA-C; canonical:3′ NTA-U; 3′ NTA-G:3′ NTA-C; 3′ NTA-G:3′ NTA-U; 3′ NTA-G:3′ NTA-A; 3′ NTA-C:3′ NTA-U; 3′ NTA-C:3′ NTA-A; and 3′ NTA-U:3′ NTA-A; canonical:5′ trimmed; canonical:3′ trimmed; 5′ trimmed:3′ NTA-C; 5′ trimmed:3′ NTA-U; 5′ trimmed:3′ NTA-A; 5′ trimmed:3′ NTA-G; 3′ trimmed:3′ NTA-C; 3′ trimmed:3′ NTA-U; 3′ trimmed:3′ NTA-A; 3′ trimmed:3′ NTA-G; 3′ trimmed:5′ trimmed; extended and 3′ NTA-A; extended and 3′ NTA-G; extended and 3′ NTA-C; extended and 3′ NTA-U; extended and 5′ trimmed; extended and 3′ trimmed; 5′ or 3′ trimmed and 5′ or 3′ extended; (trimmed and extended) and 3′ NTA-U; (trimmed and extended) and 3′ NTA-A; (trimmed and extended) and 3′ NTA-G; (trimmed and extended) and 3′ NTA-C; (trimmed and extended) and canonical. Preferably, the ratio is selected from canonical:a 3′ NTA or a 3′ NTA:a different 3′ NTA. It is clear to a skilled person that the numerator and denominator in the ratios can be reversed. Preferably, the ncRNA is miRNA.

In exemplary embodiments, the ratio is selected from canonical:3′ NTA-A; canonical:3′ NTA-G; canonical:3′ NTA-C; canonical:3′ NTA-U; 3′ NTA-G:3′ NTA-C; 3′ NTA-G:3′ NTA-U; 3′ NTA-G:3′ NTA-A; 3′ NTA-C:3′ NTA-U; 3′ NTA-C:3′ NTA-A; and 3′ NTA-U:3′ NTA-A. It is clear to a skilled person that the numerator and denominator in the ratios can be reversed.

The present disclosure demonstrates that the predictive power of an ncRNA biomarker can be improved by looking at the ratio of two varieties of the ncRNA. Example 2 demonstrates that the canonical sequence of four different miRNAs fails to discriminate between healthy tissue and renal cancer. However, clinical relevant groups can be discriminated for these same four miRNAs when the ratio of 3′ NTA-A:canonical miRNA is used.

Accordingly, the present disclosure also provides methods for classifying the health status of an individual and for collecting data regarding the health status of an individual using a small non-coding RNA (ncRNA) biomarker pair. The ncRNA pair may be based on any known ncRNA biomarker or those identified, e.g., by the methods described herein. Preferably, each ncRNA biomarker is at least 16 nucleotides in length. Various ncRNA molecules (in particular) miRNAs have been suggested for use as biomarkers (see, e.g., WO 2009099905 for markers of melanoma, WO2011025919 for markers of lung disease; WO2013003350 for markers of Alzheimer's disease; US2012289420 for markers of airway diseases, etc.). Any of these ncRNA molecules may be used in an embodiment of the methods.

The method for classifying the health status comprise determining the ratio of a biomarker pair in a bodily fluid sample from an individual and comparing the ratio to the ratio of the biomarker pair in a bodily fluid from a reference sample, wherein the presence or absence in a difference in the ratio (i.e., an altered ratio) between the individual and the reference sample classifies the health status of the individual.

The reference sample may have been obtained from a single individual or from the average of a population of individuals. The ratio for the reference sample may be determined as described herein or it may be information previously available. An alteration in the ratio from the individual as compared to the reference sample indicates an altered health status from the reference.

The choice of the appropriate reference sample is well within the purview of a skilled person. In some embodiments, the reference sample is from individual(s) having a particular disorder and an alteration indicates that the individual is not afflicted with the disorder.

In preferred embodiments, the ratio from the individual is compared to two reference samples, the first being a “healthy” control sample and the second from individual(s) having an altered health status (e.g., at risk of developing a particular disease). A skilled person will recognize that when the individual's ratio is closer to the ratio from the “altered health status” ratio, the individual is more likely to also have the altered health status.

The biomarker pair consists of two different varieties of an ncRNA, wherein the first variety is selected from the canonical ncRNA and the ncRNA with a 3′ non-templated nucleotide addition and the second variety is the ncRNA with a 3′ non-templated nucleotide addition, wherein, when ncRNA is miRNA, the first variety is selected from canonical ncRNA, the ncRNA trimmed at the 5′ or 3′ end, and the ncRNA with a 3′ non-templated nucleotide addition, optionally trimmed at the 5′ or 3′ end and the second variety is selected from ncRNA trimmed at the 5′ or 3′ end and ncRNA with a 3′ non-templated nucleotide addition, optionally trimmed at the 5′ or 3′ end. The determination of the ratio and the preferred RNA varieties are as previously described herein. Preferably, the biomarker pair consists of two different varieties of an ncRNA, wherein the first and second varieties are selected from the canonical ncRNA; the ncRNA trimmed at the 5′ or 3′ end and/or extended at the 5′ or 3′ end; and the ncRNA with a 3′ non-templated nucleotide addition, optionally trimmed at the 5′ or 3′ end or extended at the 5′ or 3′ end;

wherein, when ncRNA is not an miRNA, the first variety is selected from the canonical ncRNA and the ncRNA with a 3′ non-templated nucleotide addition and the second variety is the ncRNA with a 3′ non-templated nucleotide addition.

In preferred embodiments, the methods disclosed herein determine the likelihood that an individual will respond to a particular treatment. Preferably, the methods further comprise the step of treating the individual for the disease or disorder. In preferred embodiments, the methods determine the risk that an individual will develop a particular disorder.

In preferred embodiments, the methods disclosed herein diagnosis the individual with a disease or disorder. In preferred embodiments, the data collected can be used as one factor for assisting a medical practitioner in making a diagnosis or prognosis. Preferably, the methods further comprise the step of treating the individual for the disease or disorder.

In preferred embodiments, the disorder is prostate cancer and the biomarker pair is selected from Table 12 (from Example 3) or Table 2. In preferred embodiments, the disorder is cHL and the biomarker pair is selected from Table 4 or Table 5. In preferred embodiments, the disorder is breast cancer and the biomarker pair is selected from Table 6. In preferred embodiments, the disorder is Alzheimer's disease and the biomarker pair is selected from Table 9.

The disclosure further provides for methods for characterizing the status of classical Hodgkin's lymphoma (cHL) in an individual and for collecting data regarding the health status of an individual. The methods comprise determining the quantity of one or more miRNAs from a bodily fluid sample from the individual and comparing the amount of the one or more miRNAs to a reference sample. The quantity of miRNAs may be determined as already disclosed herein.

Example 6 describes the identification of miRNAs useful in classifying cHL. Accordingly, one or more miRNAs useful in these methods are selected from Table 3. Preferably, the one or more miRNAs are selected from let-7a-5p, miR-1908-5p, miR-5189-5p, miR-92b-3p, miR-425-3p, miR-625-3p, miR-7706, miR-125b-5p, miR-760, miR-21-5p, miR-122-5p, more preferably from miR-21-5p, let-7a-5p, miR-127-3p, and miR-155-5p.

The reference sample may have been obtained from a single individual or from the average of a population of individuals.

In some embodiments, the reference sample is from one or more healthy individuals (i.e., individuals not afflicted with cHL), one or more individuals having cHL, the same individual prior to receiving treatment for cHL, one or more individuals having a good response to treatment for the disorder, or from one or more individuals having a poor response to treatment for the disorder. For example, the quantity of one or more miRNAs as compared to reference levels obtained from healthy or afflicted individuals may be useful to characterize whether the individual is afflicted with cHL. The quantity of one or more miRNAs after treatment as compared to the levels in the individual prior to treatment may be useful to characterize the response to treatment, for example, if the treatment was effective or if the patient has re-lapsed. The quantity of one or more miRNAs after treatment as compared to reference levels obtained from patients known to be either good or poor responders to treatment may be useful to characterize whether the individual has a good prognosis for treatment.

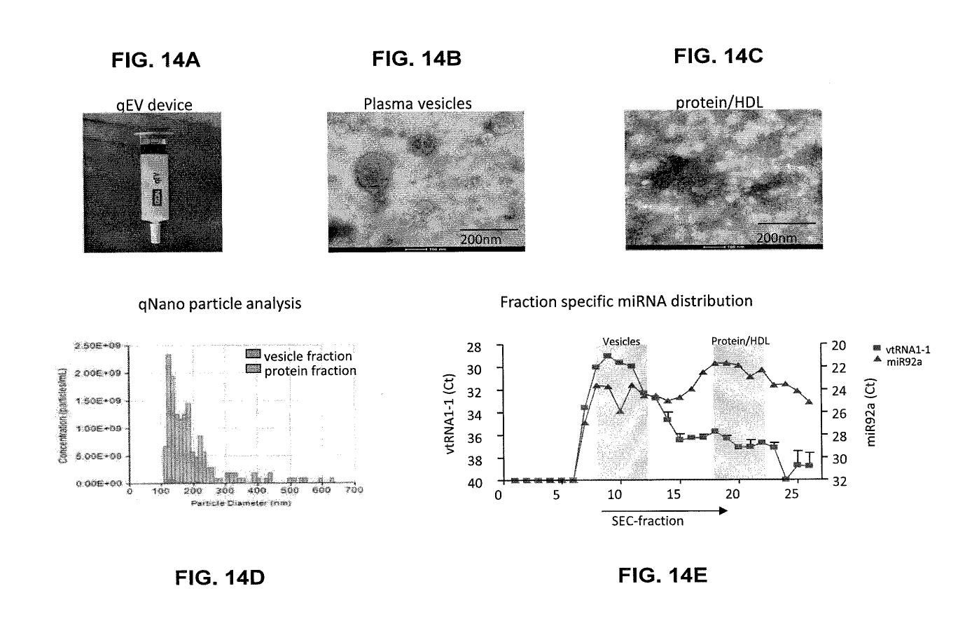

In a further aspect of the disclosure, the identification of biomarkers/biomarker pairs as well as to the characterization of a health status, the biomarkers/biomarker pairs could be improved by purifying the bodily fluid, i.e., separating the fluid into different biochemical fractions. The majority of extracellular ncRNAs in blood are associated with soluble biochemical fractions including protein-complexes, lipid vesicles (LDL and HDL) and extracellular vesicles. In preferred embodiments, the bodily fluid is enriched for either extracellular vesicles (i.e., exosomal vesicles) or for a protein-rich (protein/HDL) fraction, preferably using size exclusion chromatography. FIG. 14E depicts the separation of exosomal vesicles and the protein-rich fraction using size exclusion chromatography and an example of the difference in miRNA distribution between these two fractions. Table 4 further discloses biomarker pairs that can distinguish the exosomal vesicle fraction in blood between healthy donors and cHL patients. Table 5 discloses biomarker pairs that can distinguish the protein fraction in blood between healthy donors and cHL patients.

All patent and literature references cited in the present specification are hereby incorporated by reference in their entirety.

The disclosure is further explained in the following examples. These examples do not limit the scope of the invention, but merely serve to clarify the invention.

### EXAMPLES

### Example 1

Epstein Barr Virus (EBV)-transformed lymphoblastoid B cells (LCLs), constitutively secrete large quantities of endosome-derived extracellular vesicles (EV) called exosomes that incorporate human and viral miRNAs. Copy number measurements demonstrated that exosomes mediate cell-cell transmission of miRNAs leading to accumulation in recipient cells and target mRNA repression (Pegtel et al., 2010). Increasing in vitro and in vivo data suggest that miRNA sorting at endosomal membranes supports the function of exosomes in cell-cell communication (van Balkom et al., 2013; Kosaka et al., 2010; Mittelbrunn et al., 2011; Montecalvo et al., 2012; Pegtel et al., 2010; Umezu et al., 2013; Zhang et al., 2010; Zhuang et al., 2012). Notably, interfering with exosome formation at endosomal membranes reduces functional miRNA secretion (Kosaka et al., 2013; Mittelbrunn et al., 2011). Although secretion of miRNAs through EVs may occur in a non-random fashion (Bellingham et al., 2012; Guduric-Fuchs et al., 2012; Nolte-'t Hoen et al., 2012; Palma et al., 2012), a thorough comparative analysis in purified exosome populations and the producing cells has been lacking.

Small non-coding RNA families are differentially distributed between B cells and exosomes.

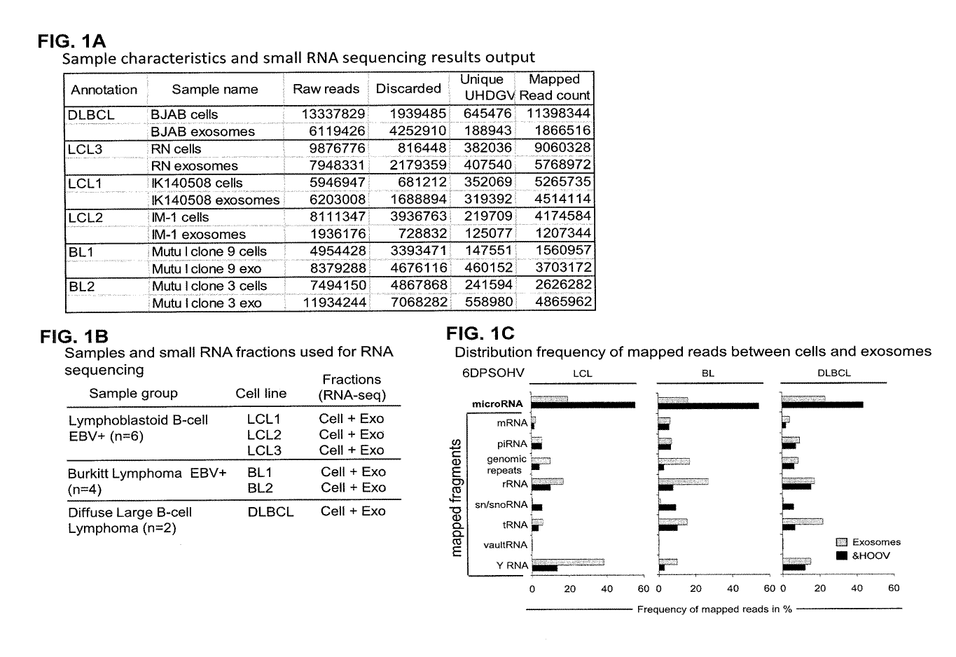

To determine whether small RNA variety of less than 200 nucleotides undergo selection for incorporation into exosomes, cellular and corresponding exosomal small RNA libraries were generated for sequencing of EBV-driven LCLs (n=3) and three lymphoma cell lines (n=3). This approach provided a comprehensive dataset to perform high-powered statistical analysis on the intra- and extra-cellular RNA repertoires within a defined cellular background. RNA-Seq yielded >1 million genome-mapped reads per sample (individual reads and alignment statistics in FIGS. 1A-1C) that were aligned to both the human and EBV genomes. Comparison to RNA reference libraries revealed that cellular and exosomal small RNA fractions contained products from diverse classes of RNAs. Besides a large diversity of small RNA sequences, it seems that several RNA classes are enriched in cells (FIG. 1C; black bars), while others are over-represented in exosomes (gray bars). In all transformed B cell types, the class of miRNAs (miRBase v19) represents around 50% of the small RNA pool. In exosomes, however, this percentage was only 20% indicating that miRNAs are retained in cells. RNA elements derived from the other ncRNA classes (i.e., tRNAs, piRNAs, rRNAs, Y RNAs, and vault RNAs) were generally enriched in the exosomes, even though the class distribution was distinct between cell types (FIG. 1B). Notably, the expression of tRNAs and tRNA-derived fragments are deregulated in diffuse large B cell lymphomas (DLBCL) (Maute et al., 2013) and other tumor tissues (Lee et al., 2009b). In DLBCL-derived exosomes, tRNA-derived fragments were highly enriched compared to LCL-derived exosomes (24% vs 7.4%; FIG. 1B). The most striking distinction between LCL and lymphoma exosomes was the extreme abundance of human Y RNAs fragments (38%; FIGS. 1A-1C). To investigate whether the observed read abundance of small RNA fragments (FIGS. 1A-1C) reflects the presence of full-length transcripts, semi-quantitative stem-loop RT-PCR was performed on the exosomal RNA that was subjected to sequencing. High levels of intact Y RNA variety (i.e., RNY1, RNY3, RNY4 and RNY5) and vault RNA1-1 (FIGS. 1A-1C) were detected in exosomes. Intact Y RNA and vault RNAs were also detected in vesicle fractions isolated from human urine samples (I. V. B., D. K-L, and D. M. P., unpublished data). This data supports a previous suggestion that small cytoplasmic regulatory RNAs may have a non-cell autonomous function (Nolte-'t Hoen et al., 2012). Overall, a consistent bias toward exosomal incorporation of RNA polymerase III-derived Y RNA transcripts and their fragments were uncovered that seem most pronounced for transformed B cells. Interestingly, human small RNAs, specifically Y RNAs are packaged in enveloped and non-enveloped viral particles (Garcia et al., 2009; Khan et al., 2007; Routh et al., 2012). The selective recruitment of small Pol III RNAs from the host cell into (retro) viral particles and exosomes implies that common molecular mechanisms drive the packaging process. This realization could aid future characterization of cellular components governing exosome biogenesis and cargo selection (Koppers-Lalic et al., 2012).

High and low abundant microRNAs are non-randomly incorporated into exosomes.

While the transcription and biogenesis of miRNAs is well-documented (Ebert and Sharp, 2012), understanding the mechanisms controlling miRNA turnover, subcellular trafficking, and release via exosomes, and how these various factors affect gene-regulatory activity remain incomplete (Ameres and Zamore, 2013).

To gain more insight into possible disparities between miRNA distribution in cells versus exosomes, Spearman correlations were calculated between normalized miRNA reads of all cellular samples and paired exosomes (n=12) and determined the variation among datasets. Cluster analysis separated the LCL samples from the lymphoma samples. For all samples, Spearman correlations (r) ranged from 0.48 to 0.81. The LCL exosomes (r=0.76 to 0.8) and LCL cells (r=0.72 to 0.76), showed relatively modest sample variability indicating their usefulness as biological replicates. Next, mapped miRNA counts were fitted in a generalized linear model using the R package edgeR (Robinson et al., 2010) and the relative abundance of individual miRNAs in cells and exosomes were compared. The most abundant cellular miRNAs (>10.000 reads per million (RPM)) in general represented the most abundant miRNAs in the exosomes (Table S2, data 1). Although the miRNA distribution in exosomal fractions is clearly influenced by cellular miRNA abundance, a subset of miRNAs was identified that were discordantly distributed between cells and exosomes (false discovery rate FDR=0.05.

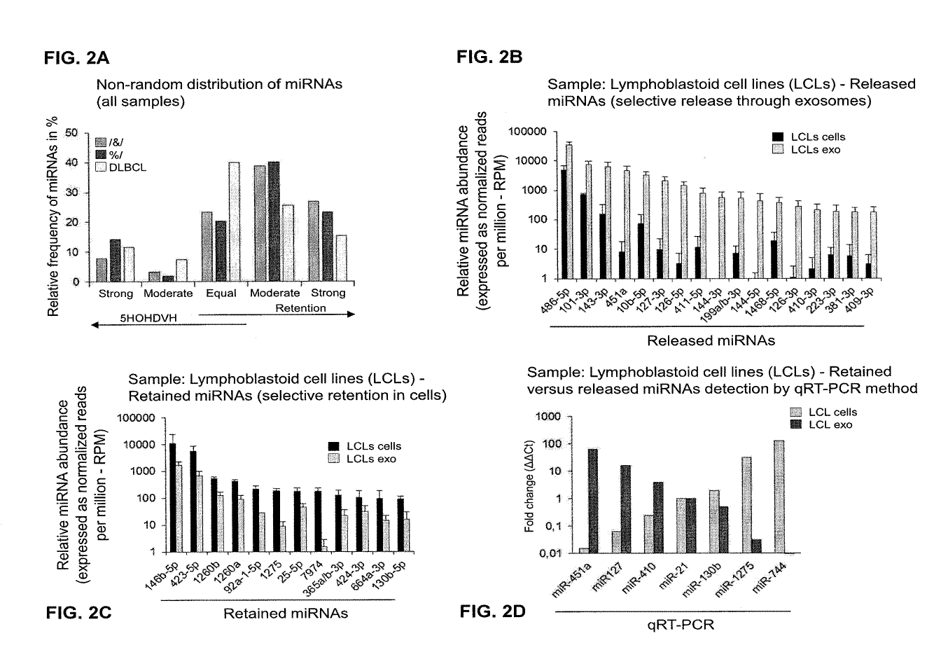

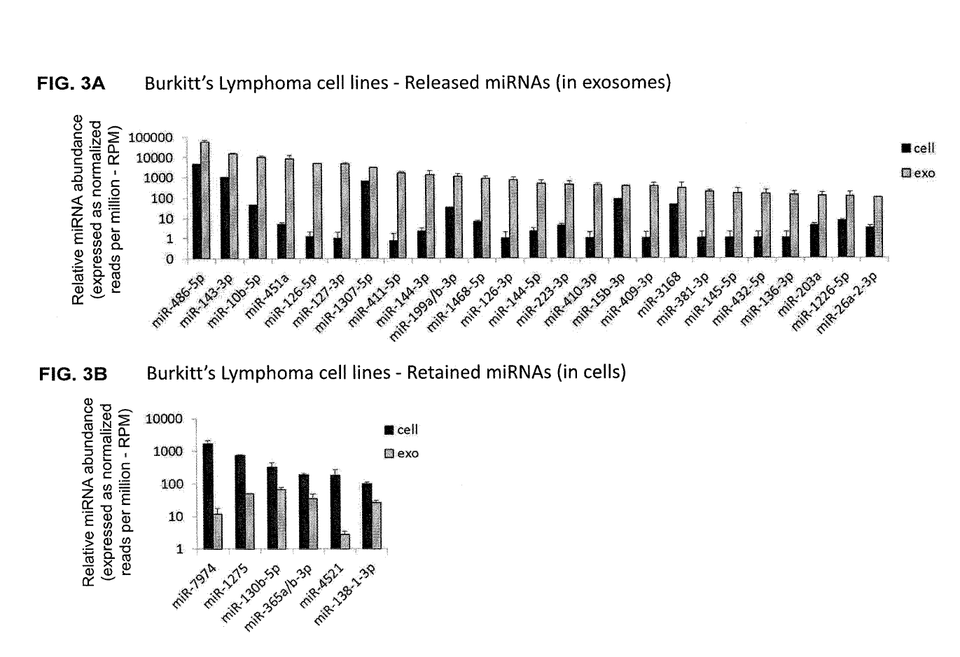

To investigate which miRNAs are preferentially retained or released in LCL, Burkitt Lymphoma (BL), and DLBCL backgrounds, all miRNAs were grouped and ranked according to the fold enrichment in exosomes versus cellular miRNA reads (RPM). It was observed that the most discordantly distributed miRNAs were preferentially retained (15% to 42%) rather than preferentially released (8% to 15%) (FIG. 2A). The recurrence of retained and released miRNAs in their cellular vs. exosomal fractions demonstrated that the number of released miRNAs shared by all sample groups is significantly different than could be expected from chance (z-score=26.2). Importantly, many “released” miRNAs have a high relative abundance of more than 100-1000 RPM (FIGS. 2B and 2C) that is sufficient for repressive gene-regulatory activity (Mullokandov et al., 2012).

To assess whether the distribution of miRNAs defined as “retained” or “released” is detectable by other methodologies, new batches of B cells secreted exosomes (biological duplicates) were prepared and extracted RNA from cells and exosomes. Next, stem-loop-based quantitative RT-PCR analysis was performed and it was observed that released human miRNAs miR-451, miR-127-3p and miR-410 were highly abundant in exosomes fractions consistent with the RNA-seq approach. Strikingly, miR-451 and 127-3p were barely detectable in cells, in contrast to preferentially “retained” miRNAs miR-1275, miR-744 and miR-130b (FIG. 2D). The expulsion of miR-127-3p could be related to its function as a negative regulator of B-cell lymphoma 6 (Bcl6) mRNA, a transcriptional repressor in lymphomagenesis (Lujambio and Esteller, 2007; Parekh et al., 2007). Indeed, miR-127-3p shows tumor-suppressive activity (Saito et al., 2006) and its release could favor the growth and survival of B cell lymphoma cells.

Differences in Human and Viral miRNA Distribution

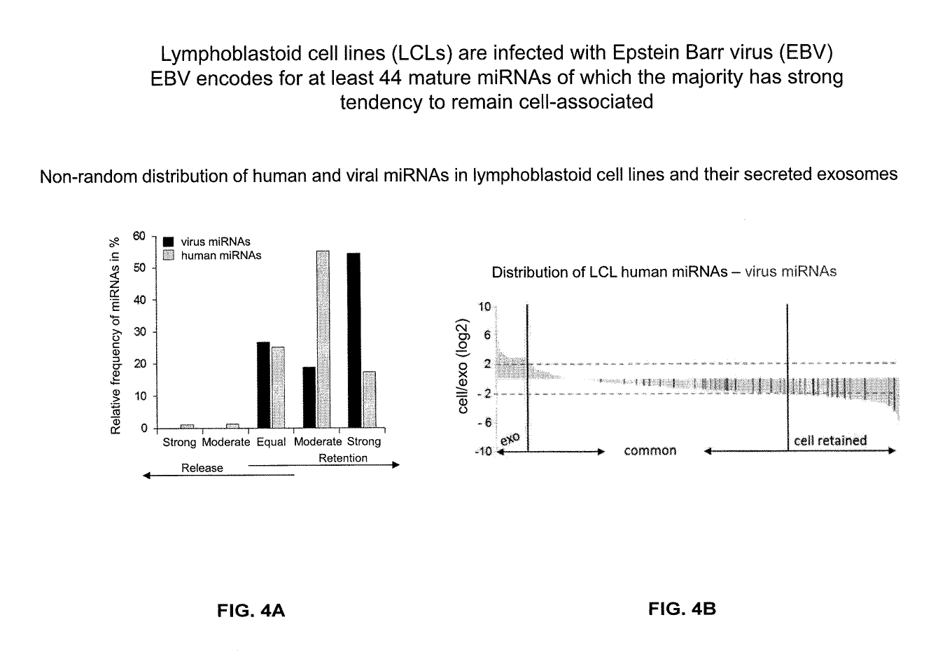

EBV-miRNAs provide essential growth advantages to EBV-infected proliferating B cells and EBV-driven lymphomas (Feederle et al., 2011; Seto et al., 2010; Vereide et al., 2013). It was postulated that, in contrast to endogenous tumor-suppressor miRNAs, EBV-miRNAs would be preferentially retained and underrepresented in exosomes. Distribution was grouped and analyzed of 44 miRNAs encoded by EBV in the transformed B cells and lymphoma cells (Qiu et al., 2011). In line with previous RNA-seq studies on EBV-infected B cell lines (Riley et al., 2012; Skalsky et al., 2012), it was confirmed that EBV-miRNA abundance varies widely and the most abundant ones belonged to the BART-cluster miRNAs. The ratio between the amount of all miRNAs released from the cells and the amount retained in the cell was calculated, after read normalization based on the frequency of their distribution (FIG. 4A). Whereas host miRNAs are prone for release (17% are cell-retained), the majority of viral miRNAs are precluded from secretion (54% are strong-retained). This is illustrated in FIG. 4B where the viral miRNAs encoded by EBV are indicated in red. This observation reconciles results from Riley et al. who found that the abundant EBV BART cluster miRNAs target 132 apoptosis-related cellular mRNAs (Riley et al., 2012), suggesting that EBV miRNAs prevent cell death. Gene ontology analysis indicated that many host miRNAs known to repress the MAPK/PI3K survival pathway are preferentially released. The observation of a coherent, non-random distribution of physiologically relevant miRNAs is consistent with a mechanism that mediates sorting into exosomes.

3′ End NTAs Define Retention or Release of miRNAs In Vitro and In Vivo

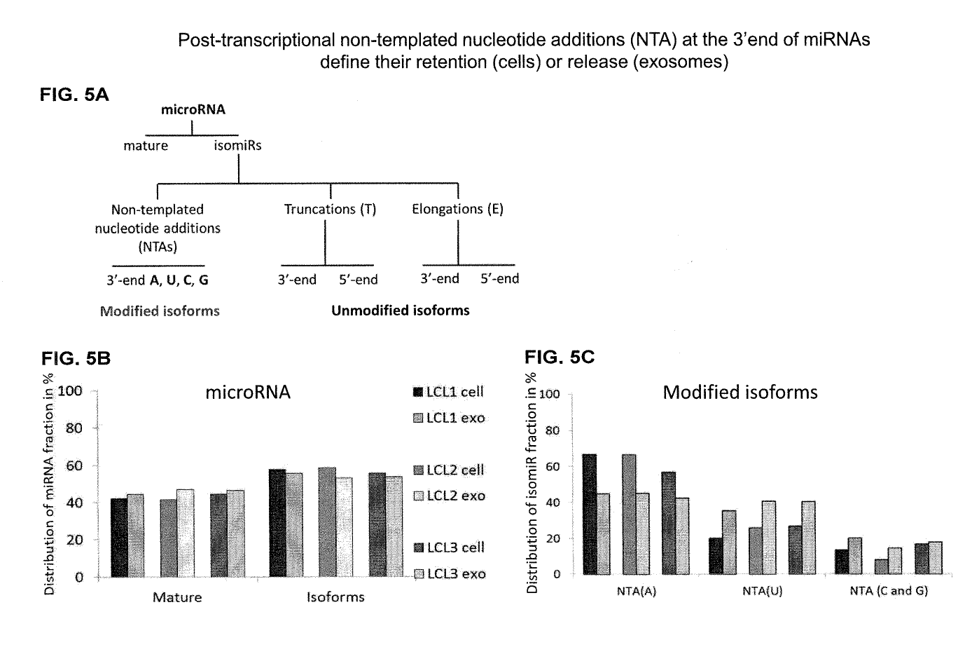

The most abundant miRNA sequences from pre-miRNA hairpins are normally used to define the corresponding miRNAs, commonly termed as mature miRNAs (miRBase). However, the analysis of individual miRNA reads and their mappings to the corresponding pre-miRNA sequence, indicated extensive sequence variations at the 3′ end of miRNAs in cellular and exosomal RNA fractions. Inefficient Drosha or Dicer-mediated processing of pre-miRNAs produces 3′ end length variants (e.g., truncations and elongations). Additionally, miRNA isoforms are generated actively by nucleotidyl transferase-mediated post-transcriptional modifications, also referred to as “non-templated terminal nucleotide additions” (NTA) (Burroughs et al., 2010; Morin et al., 2008; Wyman et al., 2011) (FIG. 5A). Global analysis of miRNA 3′ end variations showed that 3′ end length isoforms are equally distributed between cells and exosomes, while miRNAs with 3′ end NTAs exhibited differential distribution. The comparison of individual NTA types (i.e., A, U, C or G) to the sum of all reads with NTAs showed an enrichment of adenylated miRNA isoforms in the cellular fractions. In contrast, 3′ end uridylated miRNAs were overrepresented in exosomes of all LCL samples analyzed (FIG. 5C). To analyze whether the proportion of NTA-modified reads (A, U, C or G) changes significantly between cell and exosome samples, each miRNA sequence with 3′ end NTAs was extracted from all 12 samples (LCLs n=6, BLs n=4, DLBCL n=2) and fitted in a logistic model, which also corrected for the cell line effect (accounting for the paired design). The results suggest that 3′ end adenylated miRNA isoforms are preferentially retained (chi-square test, p<0.05), while all other NTAs are preferentially released (3′ end-U: chi-square test, p=0.02; 3′ end-C: Fisher's exact test, p=0.04; and 3′ end-G: Fisher's exact test, p=0.02.

Apart from a global NTA distribution analysis, in three LCL samples (i.e., biological triplicates), it was observed that the disparity of 3′ end adenylated or uridylated miRNA isoforms between cells and exosomes was significant (p=0.005, student t-test) and not observed for mature miRNA (miRBase-based annotation) sequences or unmodified (elongated or truncated) isoforms. To analyze the effect of 3′ end NTAs on distribution of individual miRNAs, 100 abundantly expressed miRNAs in LCL cells were selected and matched with exosome fractions for a pair-wise analysis. The results demonstrated that sequences of individual miRNAs with 3′ end adenylation are more associated with cellular fractions, whereas the same miRNAs but with 3′ end uridylation are more prone to exosomal release. It was concluded that the type of added nucleotide, i.e., adenine or uridine, influences miRNA isoform distribution between cells and exosomes.

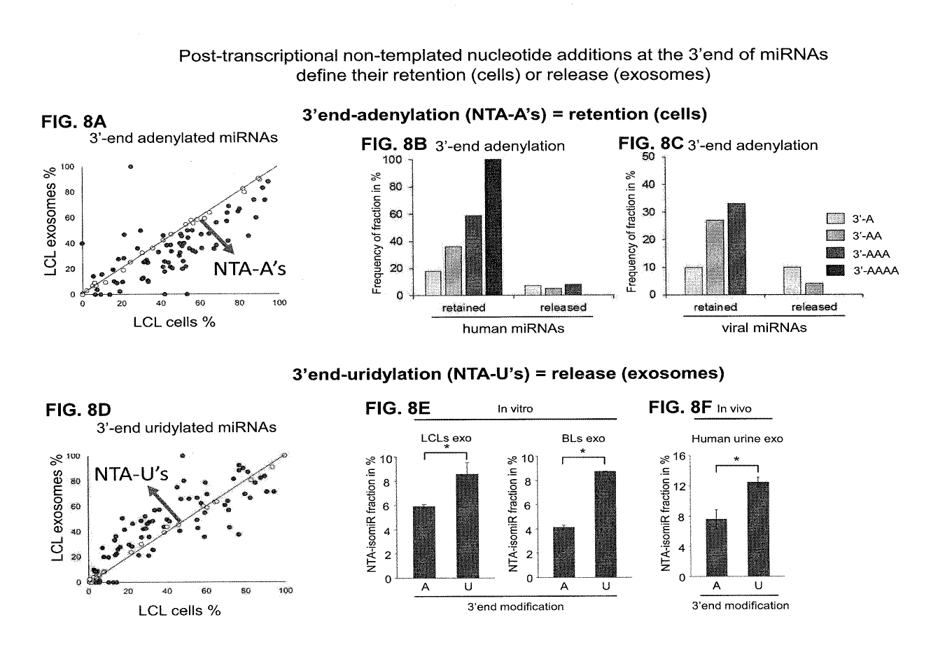

Deeper analysis of individual miRNA sequences allowing more mismatches and longer flanking regions (+6 nt) outside defined and annotated 3′ end sequence revealed that 3′ ends of many miRNAs are extended with more than one non-template nucleotide. To address whether the number of post-transcriptionally added nucleotides has a cumulative effect on the distribution, the ratio of the fold-changes between cells and exosomes was first calculated (defined as Expel Coefficient; see experimental procedures) for sequence reads with a particular NTA type with non-modified forms, taking into account the number of added nucleotides. 3′ end adenylation increases the probability for retention and this probability increases proportionally with the number of added A's (FIGS. 8B and 8C). Strikingly, tri-adenylated human miRNAs were five times more frequent in LCL cells than in the corresponding exosomes (p=0.03; unpaired t test), whereas tri-adenylated viral miRNAs are only detected in LCL cell fractions (FIG. 8C). The association of 3′ end adenylation with an increase in cellular retention of viral miRNAs further corroborates the observation that viral miRNAs exhibit strong tendency to remain cell-associated (FIGS. 4A and 4B). Importantly, extensive 3′ end poly-adenylation (more than four adenines) that signals for miRNA degradation as recently demonstrated by Backes et al. (Backes et al., 2012) was not observed.

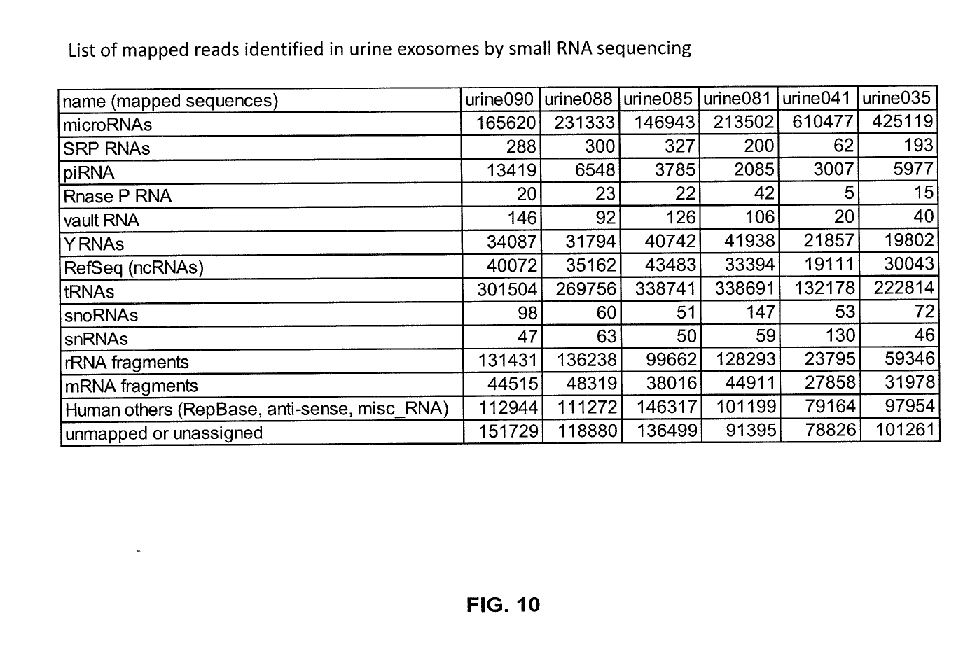

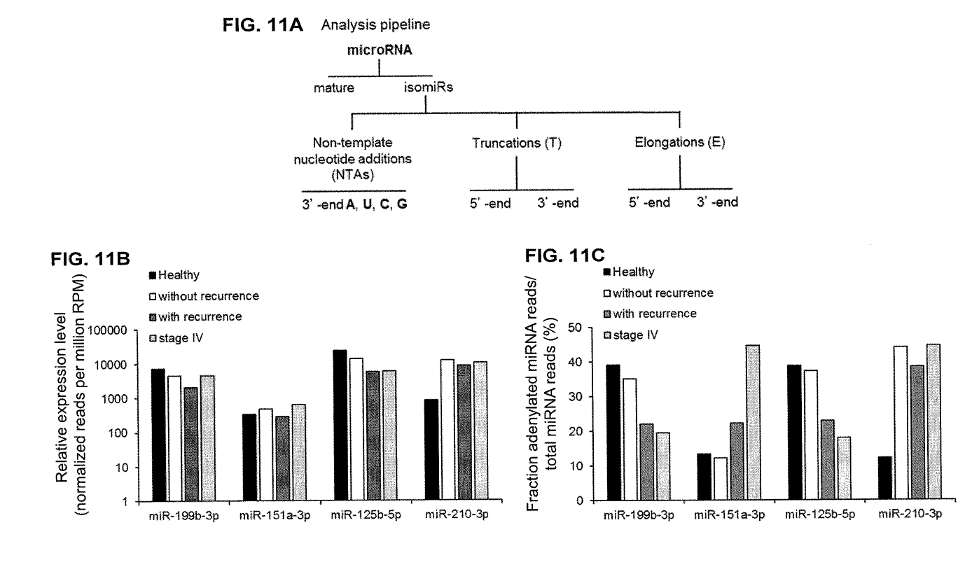

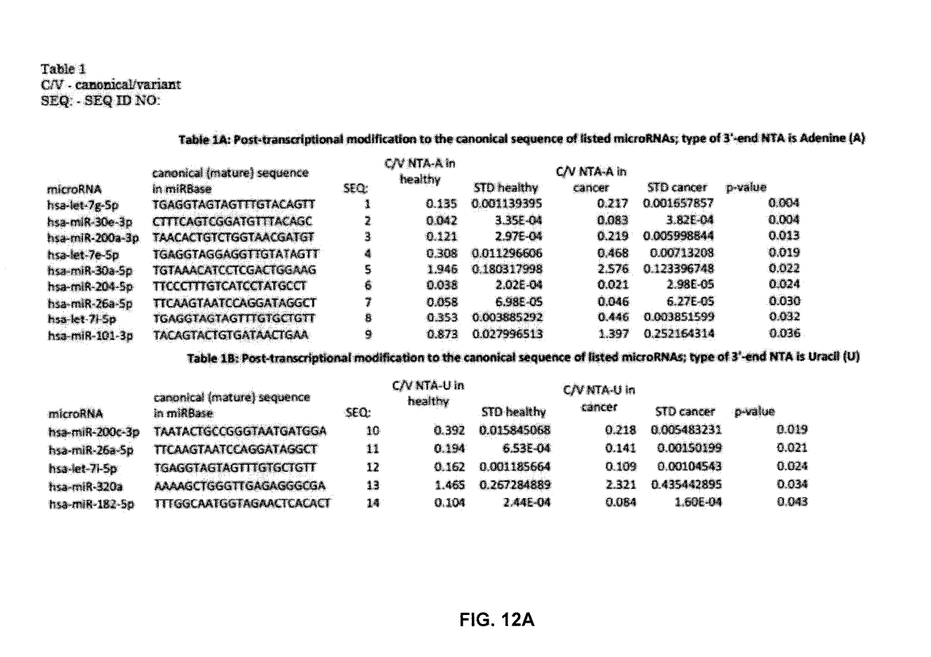

In contrast to 3′ end adenylation, the disproportional occurrence of small RNAs with uridines at the 3′ ends seems to define exosomal RNA cargo. To investigate whether or not this is a phenomenon restricted to B-cells in culture (FIG. 8F), naturally occurring extracellular vesicles were collected and purified from human urine using an exosomes-isolation protocol (Bijnsdorp et al., 2013; Verweij et al., 2013). The small RNA content was sequenced from six urine exosome samples derived from healthy individuals (FIG. 10). In full accordance with 3′ end uridylation-mediated miRNA isoforms, distribution was observed in B cell exosomes (FIG. 8F; p-value p<0.005; Student t-test) and human urine exosomes were significantly enriched in 3′ end uridylated miRNAs (FIG. 8F; p-value <0.0001; Student t-test). Collectively, these data indicate that in vitro, as well as in vivo, 3′ end uridylated miRNAs are preferentially released by cells, and that cellular retention of miRNA isoforms depends on the number of adenine nucleotides added to the 3′ end of the miRNA.

The Impact of 3′ End NTAs on Exosomal Sorting of Individual miRNAs

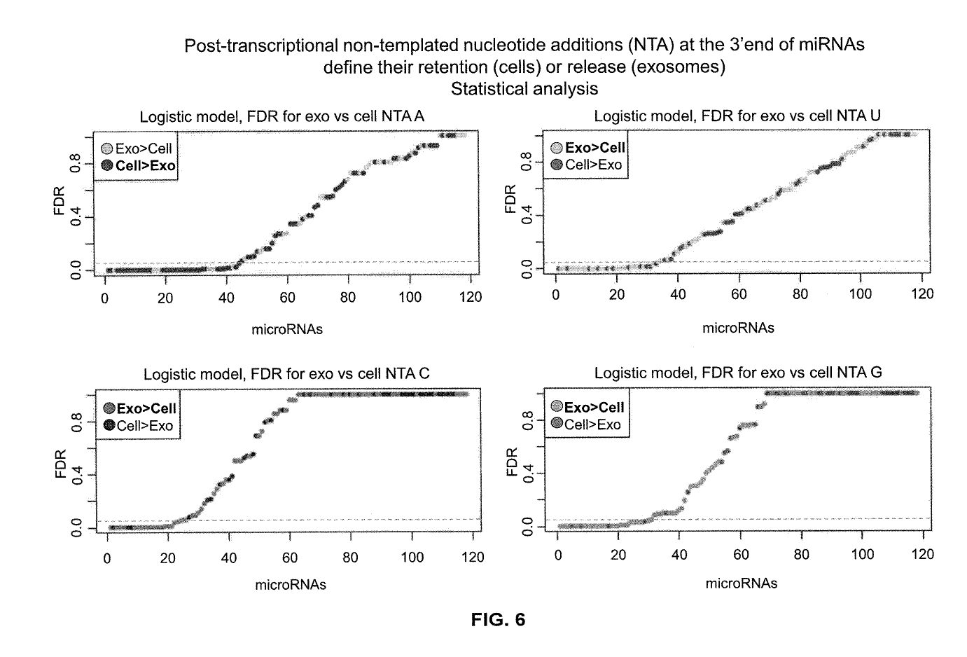

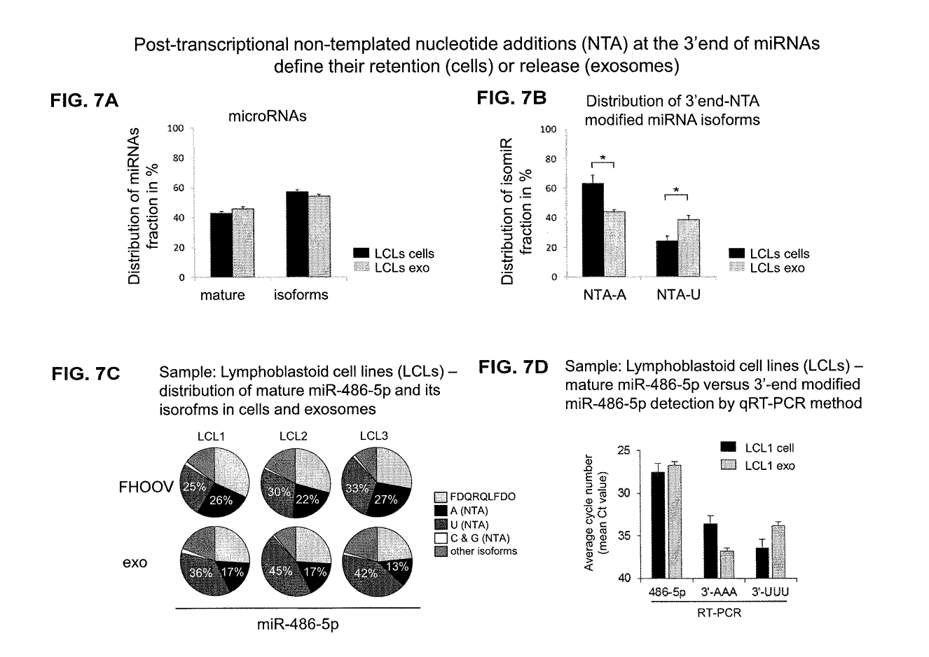

Our results showed that NTAs define miRNA distribution between cells and exosomes. However, the impact of NTAs on individual miRNAs may be distinct. For example 3′ end uridylated reads of abundant exosome-enriched miRNAs 486-5p, 143-3p and 101-3p show an above average percentage (38%) of all uridylated isoforms present in exosomal fraction. Yet, miR-486-5p is seemingly equally distributed at the mature miRNA level (FIG. 7C; in gray). Notably, 50% of all reads mapping to the mature miR-486-5p sequence are either adenylated or uridylated isoforms (FIG. 7C). When using the weighted means of all miRNAs in LCL samples, it was observed that tri-uridylated reads are nine times more frequent in LCL exosomes compared to the cellular content (p=0.01; unpaired t-test). To confirm this difference in distribution, stem-loop-based primers were developed and designed to distinguish miR-486-5p 3′ end tri-adenylated and 3′ end tri-uridylated isoforms that are three nucleotides longer than the canonical (mature) miRBase-based sequence. As expected, the detection of mature miR-486-5p with no 3′ end nucleotide additions shows a virtually equal distribution between cells and exosomes, whereas 3′ end tri-adenylated isoforms were enriched in cells (ten-fold) and 3′ end tri-uridylation (five-fold) in exosomes (FIG. 7D). This independent approach supports the prior RNAseq data (FIG. 6) and demonstrates that different types of 3′ end post-transcriptional modifications are detectable by RT-PCR and reflects a cellular retention and exosomal sorting process. Thus, abundant miRNAs and their isoforms represent subpopulations with unique distribution properties depending on the type and the extent of 3′ end post-transcriptional modification.

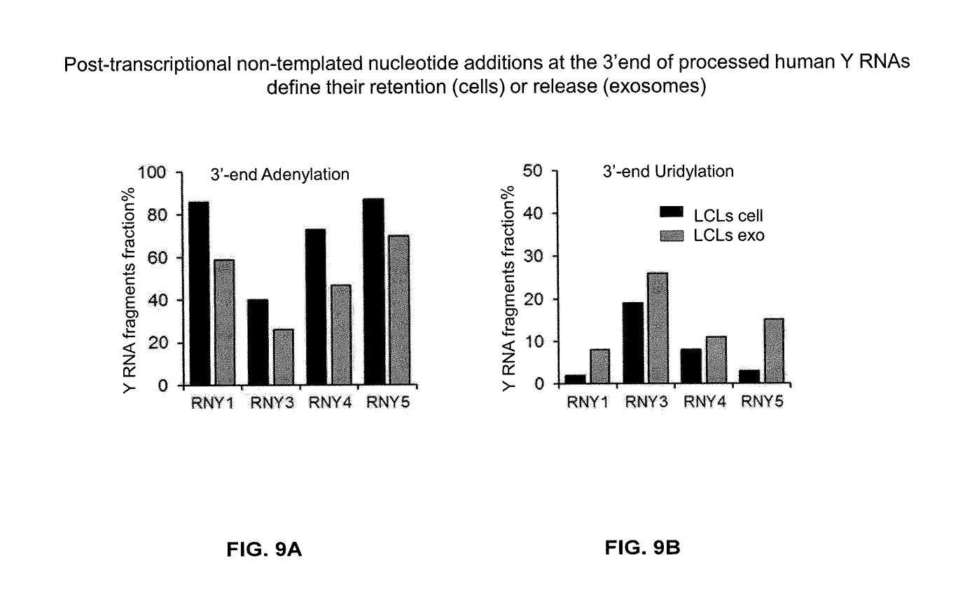

Apart from human and viral miRNAs, it was found that adenylation and uridylation can also occur at the 3′ ends of processed fragments originating from small cytoplasmic Y RNAs (FIGS. 9A and 9B). Importantly, most of the 3′ end modified small RNA molecules followed the same fate as miRNA isoforms with regard to exosomal sorting, indicating that NTA modifications as a signal for sorting is not restricted to the class of miRNAs. These results underscore the relevance of 3′ end NTA and expand the knowledge on diversity of small RNA substrates for the nucleotidyl-transferase family of enzymes (Martin and Keller, 2007).

The comparison between cellular and exosomal miRNA repertoire demonstrated conclusive evidence for selective exclusion and release of abundant miRNAs via exosomes. Moreover, it was observed that the degree of 3′ end adenylation seems to impede the incorporation and secretion of miRNA isoforms via exosomes. This finding is consistent with the notion that this particular type of modification increases the stability and activity of certain miRNAs (Burns et al., 2011; D'Ambrogio et al., 2012; Jones et al., 2009). Importantly, an opposite behavior for 3′ end uridylated miRNA isoforms was found. It was concluded that miRNA trafficking, sorting, and release via exosomes, which is also evident in naturally occurring human urine vesicles, is defined in part by post-transcriptional modification through NTA. Currently, the functional significance of these subtle 3′ end modifications are becoming unraveled (Baccarini et al., 2011; D'Ambrogio et al., 2012; Jones et al., 2009; Katoh et al., 2009; Rüegger and Groβhans, 2012; Scott and Norbury, 2013), although their exact role in selective miRNA turnover, abundance, and activity is far from understood and complex (Ameres and Zamore, 2013). The study provides a further understanding of exosomal small RNA cargo selection and offers a rationale for studying miRNA processing, modification, and turn-over in connection to exosome biology.

### Experimental Procedures

### Preparation of RNA Samples for Deep-Sequencing

For each cellular or exosomal sample, an equal amount of input RNA (600 ng of RNA) was prepared for sequencing using the TRUSEQ® small RNA sample prep following the manufacturer's instructions (Illumina San Diego Calif. USA). Sequence libraries were measured on an AGILENT® 2100 BIOANALYZER® (Agilent, Santa Clara Calif. USA) and up to 12 samples were equimolarly combined per run. Sequencing was performed on a HISEQ® 2000 (Illumina San Diego Calif. USA) paired end 100 cycle (PE100) run.

Detection of RNA Reads with Non-Templated Nucleotides Additions and Statistical Analysis

MiRNA isoforms or isomiRs are retrieved by step-wise analysis. Briefly, for detection of NTAs at 3′-termini of mapped RNA elements, the read sequence was compared to the aligned pre-miRNA sequence, starting at the nucleotide position 18 of the read. If the algorithm finds a mismatch position between the read and the pre-microRNA after position 18, the read is further analyzed from the mismatch position to the end of the read while all following nucleotides need be equal to the one at the mismatch position. If a different nucleotide is encountered, the search is continued at the next mismatch position or the read is tagged as non-NTA. After detecting all NTA reads, the weighted mean per sample was calculated by dividing the number of reads ending with NTAs of a given nucleobase (A, U, C and G) by total number of reads mapped to miRNAs. Per miRNA and per sample, the total number of reads ending with NTAs of a given nucleobase were computed, in addition to the total number of reads mapped to that miRNA. So, for each nucleobase, the number of reads could be used, in relation to the total, in a logistic regression model that also included the sample type (cell or exosome) as well as the cell line (accounting for the paired design). Based upon this model, p-values were extracted for the difference between proportions of miRNA counts with NTA of a given nucleotide, between cells and exosomes. After fitting this model per miRNA, Benjamini-Hochberg's false discovery rate (FDR) is applied to the p-values in order to correct for multiple testing.

Expel Tendency of Small RNAs with Non-Templated Nucleotides Additions

The assessment of the distribution of post-transcriptionally modified reads (NTAs) between cells and exosomes was performed as follows. To test if miRNA-related reads have a tendency either to be incorporated into exosomes or to be retained within cells, they were compared with those to the baseline distribution tendency of the given miRNA (defined by all other reads except the ones that belong to the analyzed isomiR type). The expel coefficient (EC) was defined by computing the ratio of the fold changes between exosomes and cells for reads ending with a certain type of non-templated nucleotide (defined as NTA) and those without terminal modification (defined as noNTA). The coefficient will be close to 0 for which the NTA-reads and the non-NTA reads of a small ncRNA sequence have the same tendency to be released or retained. It will be positive if the NTA-reads have a stronger tendency to get released compared to the non-NTA reads. Finally, a negative coefficient indicates that NTA-reads have a stronger tendency to remain within the cells compared to non-NTA reads.

$$EC = {log}_{2} ( \frac{{RPM}_{exosome}^{NTA}}{{RPM}_{cell}^{NTA}} / \frac{{RPM}_{exosome}^{noNTA}}{{RPM}_{cell}^{noNTA}} )$$

### Exosome Collection Procedures

For exosome collection, each cell line was cultured in RPMI-1640 supplemented with 10% exosome-depleted FBS for three consecutive rounds followed by harvesting exosome-containing supernatant (one collection round represents one exosome-containing preparation; 100×10⁶ cells in 200 ml of medium). Cell death was analyzed routinely by trypan-blue exclusion, and cultures with ≦90% viability were not considered for exosome collection. Exosomes were isolated and purified from the supernatants using the differential centrifugation protocol as described (Verweij et al., 2013). Briefly, the final step involved pelleting of exosomes at 70,000×g for 1 hour and pellet washing with PBS prior to last ultracentrifugation step (70,000×g). Pelleted exosomes were dissolved in 100 μL PBS and stored at −80° C. All exosomal preparations were analyzed by immunoblotting to confirm the presence and purity of exosomes (data not shown) as previously described (Pegtel et al., 2010; Verweij et al., 2011). For extraction of urine exosomes, fresh urine samples (50 ml volume) were collected from healthy males (n=6) after signed informed consent. Those healthy males are control group of patients that participate in a study unrelated to this project at the department of Urology (VU University Medical Center, Amsterdam, the Netherlands). Exosomes were isolated by differential centrifugation. First, urine was centrifuged at 500×g for 20 minutes, at 2000×g for 20 minutes, 10,000×g for 30 minutes and 100,000×g for 1.5 hours with one additional washing with PBS. Intact exosomes were collected in PBS, and treated with RNAse A (400 ng/ml, Sigma-Aldrich Chemicals, Zwijndrecht, The Netherlands) before RNA isolation.

### RNA Extraction, Quality Assessment and Semi-Quantitative PCR Assay

The RNA was extracted from cells and from paired exosome pellets. Exosome preparations were pretreated with 400 ng/μl RNase A (Sigma) at 37° C. for 1 hour. Total RNA was extracted from all exosomal preparations by TRIZOL® reagent (Life Technologies). When low yields were expected, 5 μl of glycogen (Roche) was added before the isopropanol precipitation step. To enable small RNA profile comparison between cellular samples and paired exosomes (known to contain the majority of small RNA varieties of <200 nt in size), cellular RNA was extracted by using glass fiber filter-based method (mirVana miRNA Isolation kit; Life Technologies) to enrich for small RNA varieties present in cellular samples. After extraction, all cellular RNA samples were comprised of small RNA varieties (<200 nt). Small cellular and exosomal RNA profiles exhibited similar patterns in size distribution. The quality and quantity of extracted small RNA (cellular and exosomal) was assayed using a small RNA chip platform (AGILENT® 2100 BIOANALYZER®).

Presence of full-length small non-coding RNAs in cellular and exosomal samples was detected by stem-loop-based semi-quantitative reverse transcriptase PCR (RT-PCR) with SYBR® Green detection and analyzed by LIGHTCYCLER® 480 (Roche). This method makes use of a stem-loop primer for RT reaction. Stem-loop primers are designed to complement a stretch of the last eight nucleotides at the 3′-end of small RNA of interest and used at the final concentration of 12.5 nM. TAQMAN® MicroRNA RT kit (Applied Biosystems) was used, and the reactions were incubated according to the manufacturer's instructions. Products were amplified by RNA-specific forward primers and by a reverse primer specific for the overlapping region of target and stem-loop RT primer. The analysis of amplified products was performed by LIGHTCYCLER® 480 Software (Roche). Cellular and exosomal miRNAs were detected by TAQMAN® microRNA assays and Custom TAQMAN® small RNA assays (Life Technologies) following the manufacturer's instructions. All miRNA data is obtained from samples analyzed by Applied Biosystems 7500 Fast Real-Time PCR Systems and the analysis software provided. For all RT-PCR reactions, an equal amount of RNA was used. The results are presented as averaged cycle threshold (Ct) values (mean Ct values) of two technical replicates from duplicate experiments in FIG. 4B, except for FIG. 2D, where Ct values were normalized for miR-92a using the ΔΔCt method. Samples with a Ct greater than 40 are regarded as negative.

### Analysis of Deep Sequencing Data and Expression Profiling of Small RNAs

The expression profiling of small RNAs was performed with a pipeline based on the miR analyzer program (Hackenberg et al., 2011), including additional analysis steps and customized scripts. Briefly, the preprocessing of the reads in FASTQ™ format includes: i) adapter trimming (at least 10 nt of the adapter need to be detected allowing one mismatch between the read and the adapter sequence), ii) delete cleaned reads shorter than 15 nt, and iii) collapse all reads with identical sequences into one entry (unique reads). The read count of a unique read is the number that represents how many times the corresponding RNA molecule has been sequenced.

After adapter trimming and unique reads grouping, the reads are aligned by means of the Bowtie algorithm (Langmead et al., 2009) to a pooled index of the human (GRCh37 patch release 5; downloaded as hg19 from UCSC) and EBV genome (Gene Bank Accession number NC_007605). An alignment seed length of 19 bp was used, allowing one mismatch, retrieving only those alignments that map to less than 40 positions in the genome. In order to receive only the best alignments out of all those that satisfy the criterion, a seed extension method was applied as explained by Hackenberg et al. (Hackenberg et al., 2011). Note that the Bowtie seed alignment method allows aligning only the first L bases of a read. Non-genome-templated nucleotide additions (NTA) of adenine (A), uracil (U), cytosine (C) and guanine (G) are often detected by RNA sequencing at the 3′-ends of small RNAs. The post-transcriptionally added bases are not present in the genome sequence and, therefore, would be detected as mismatches in the alignments. The seed alignment allows detection of those reads as mismatches outside the seed region and will not be accounted for the allowed mismatches. After the alignment to the genome (human and EBV genomes combined), several annotations are used for the expression profiling of small RNA varieties. In general, the read position must be completely positioned within a region defined by the small RNA annotation: [chromosome start−3 nt: chromosome end+6 nt]. Flanking regions were allowed in order to be able to detect length variants and NTAs. The expression value of a given small RNA is simply the sum of all reads that map within the corresponding genome region. Furthermore, an adjusted read count (RC) was calculated by dividing the RC of each read by the number of mapped chromosome positions. Finally, for each small RNA element, the Read Per Million expression value (RPM) was calculated, normalizing by means of the total number of reads mapped to a given RNA variety. The RPM is the most common type of normalization, making the expression value independent of the total number of reads.

To provide annotations to RNA elements that mapped to human and EBV genomes, all mapped reads were analyzed against currently known databases: 1) mature and pre-miRNA sequences from miRBase version 19 (Kozomara and Griffiths-Jones, 2011), 2) NCBI Reference Sequences human RefSeq genes downloaded Jun. 2, 2013 (Pruitt et al., 2012), 3) tRNA sequences from the genomic tRNA database (Chan and Lowe, 2009) Repeat Derived sequences detected by means of the RepeatMasker algorithm downloaded from UCSC, and 5) piwiRNA (piRNAs) downloaded from the NCBI nucleotide database. For those annotations in fasta format, the genome coordinates were obtained by mapping the sequences to the genome.

### Statistical Analysis for Comparing Cell and Exosome Samples

The RNA-seq libraries involved one pair of libraries per cell line, corresponding to either RNA coming from whole cells or RNA from exosomes only. To compare abundance for each miRNA between cell and exosome samples, the observed counts were fitted in a generalized linear model using the R package edgeR (Robinson et al., 2010), which included the cell line accounting for the paired design, as well as the sample type (either cell or exosome). The model included not only common and trend dispersion, but also tagwise dispersion estimation, allowing thus for extra variability due to inter-library fluctuation. The p-values corresponding to the likelihood-ratio test statistic for the sample type effect were corrected for multiple testing using Benjamini-Hochberg's false discovery rate (Benjamini, 2010). In addition, the observed intersection between six samples was also compared to the random expectation. Briefly, the i) log 2 ratios of expression values were calculated between exosomes and cells for all six samples, ii) all expelled (log 2>=2) miRNAs were extracted, iii) the shared miRNAs for a given group were extracted, i.e., those that are expelled in all samples of this group, iv) X miRNAs were randomly extracted 10,000 times for each condition (X is the number of observed, i.e., expelled miRNAs), v) for each of the 10,000 random runs, the number of shared miRNAs were detected, vi) from the 10,000 random runs, the mean and standard deviation of the randomly shared RNAs were calculated, which gives the expectation value and its fluctuation under random assumption, vii) by means of the observed number of shared RNAs and the random mean and standard deviation, a z-score was calculated to assess the statistical significance. Z-scores of 1.645 or higher, which corresponds to p≦0.05, were considered as significant.

### Example 2

An experiment on clear cell Renal Cell Carcinoma (ccRCC) that consists of 11 healthy (non-tumoral renal cortex—NRC) and 22 clear cell Renal Cell Carcinoma samples was selected (S. Osanto et al., PLoS One 2012). Furthermore, the 22 ccRCCs patients belonged to three prognostic sub-groups, i.e., without disease recurrence, with recurrence and with metastatic disease at diagnosis (stage IV). The data with SRA accession SRP012546 was downloaded from the Short Read Archive. To profile the miRNA expression pattern and to determine the degree of isomiRs the previously developed widely used program, miRanalyzer that annotates complex (small) RNAseq data was extended (M. Hackenberg et al., Nucleic Acid Res. 2009 and 2011). After adapter trimming, the unique reads are mapped to the human genome. To detect all sequence variants, fluctuations are allowed compared to the canonical sequence: 3 nt fluctuation at the 5′ end and 5 nt at the 3′ end.

After detecting all reads that belong to a given miRNA, the reads are hierarchically classified (assigned to an isomiR type): i) the canonical miRNA sequence, ii) non-templated additions (NTA) and iii) 5′ and/or 3′ sequence length variants (e.g., truncations or elongations) from the canonical sequence. The isomiR ratios are calculated for each miRNA and isomiR type. They are defined as the number of reads that belong to a given isomiR type divided by the total number of reads mapped to a given miRNA (canonical read count plus all isomiRs).

After analyzing the data with the pipeline, the observed isomiR ratios were compared between all four experimental groups (healthy, without recurrence, with recurrence, and stage IV). In order to obtain those miRNAs for which statistically significant differences exist between two groups, a standard t-test was applied. Pronounced differences were found for adenine additions (adenylation) between the clinically relevant groups. Four miRNAs were extracted that showed a very high percentage of adenylated reads in at least one group (>=35%) and at least one significant comparison. The four mature miRNAs are: miR-199b-3p, miR-125b-5p, miR-210-3p and miR-151a-3p. FIGS. 11A-11C depict the isomiR ratios of these four miRNAs for all four different patients groups.

### Brief Summary of Preliminary Results

1. Distinct miRNAs show a strong correlation between adenylation frequency and expression level including miR-210-3p, which corresponds to stabilization of the mature miRNA sequence (D'Ambrogio et al., Cell Repots 2012). MiR-210-3p levels are increased during RCC progression, which might be a consequence of miRNA stabilization through post-transcriptional modification in a form of 3′-end adenylation.

2. Between stage IV and healthy individuals, mature miR-199b-3p is not differentially expressed (see FIGS. 11A-11C); however, highly significant differences (p-value: 3.83E-06) exist in the adenylation pattern. Furthermore, miR-199b-3p targets four highly relevant genes for renal carcinogenesis (MED6, KRT7, MET, JUNB). This miRNA is thus putatively involved in oncogenesis, but would not be detected by traditional analysis pipelines stressing the importance to independently determine isomiR content.

3. Individuals with ccRRC that do not show recurrence after treatment manifests miR-199b-3p adenylation ratios closer to healthy individuals (35%, p-value=0.13), while those individuals with recurrence do have ratios closer to stage IV individuals (22%, p-value=4.72*10-4). For miR-125b-5p, a very similar pattern can be observed (see FIGS. 11A-11C). This suggests that isomiRs have unexpected prognostic biomarker potential.

### Example 3

### IsomiRs in Biofluids Discriminate Cancer Subtypes and Healthy Patients

Urine was collected from four healthy volunteers and nine patients diagnosed with prostate cancer. Exosomes were collected and RNA was extracted as described in Example 1. Expression profiling and data analysis of small ncRNAs was performed as in Examples 1 and 2.

After detecting all reads that belong to a given miRNA, the reads are hierarchically classified (assigned to an isomiR type): i) the canonical miRNA sequence, ii) non-templated additions (NTA), and iii) 5′ and/or 3′ trimmed variants. The isomiR ratios are calculated for each miRNA and isomiR type. The most statistically significant ratios (determined by p-value) are depicted in FIGS. 12A-12C. The ratio of variant to canonical is significant between healthy patients and those having prostate cancer.

### Example 4

### Size-Exclusion Chromatography (SEC) for Single-Step Isolation of Vesicles from Human Plasma

Measuring circulating ncRNAs, for example, miRNAs, in the blood maybe useful for early cancer detection, prognosis and monitoring. Exhaustive RNA sequencing on tissues and cultured cells reveals a complex and dynamic repertoire of ncRNAs. Over 2000 mature miRNA species have been identified, of which many have been detected in circulation and other biofluids such as saliva and urine. However, a single genomic locus can produce many functionally distinct ncRNA variants.

The majority of extracellular ncRNAs in blood are associated with soluble biochemical fractions including protein-complexes, lipid vesicles (LDL and HDL) and extracellular vesicles. Comprehensive ncRNA profiling in total serum or plasma is tedious due to technical restraints. Small RNA library preparation of circulating small RNAs and their variants requires a high-enough enrichment and purity of intact ncRNA species that are available for adapter-ligation.

In an effort to increase the signal-to-noise ratio for measuring ncRNAs relevant to an individual's heath status, miRNAs were measured in purified exosomal vesicles (EVs) and in the protein fraction as follows:

Pre-Treatment Sepharose CL-2B
Pour 20 mL of sepharose CL-2B (per column) in a beaker and let the sepharose CL-2B settle for at least 15 minutes.
Discard supernatant
Add 15 mL buffer and mix gently by swirling
Let the sepharose settle for at least 15 minutes
Repeat wash twice
Discard the supernatant and add 10 mL of buffer

Column Preparation
Use a 10 mL syringe
Place/press the nylon panty hose into the outlet of the syringe
Pipette the washed sepharose into the column (use plastic Pasteur pipette
Let the sepharose settle, avoid column from running dry; column should be nicely vertical, no angle (use level)
Add sepharose until 10 mL of stacked sepharose is reached
Avoid column from running dry. Add buffer until use or close syringe outlet with plug (use column within a few hours)

Sample Loading

Let the buffer run almost completely into the sepharose column (but prevent column from running dry)
Load 1.5 mL of plasma on the column
Immediately start collecting samples of 0.5 mL
When the plasma sample has almost completely entered the sepharose, carefully add buffer until the syringe is completely filled.
Repeat the addition of buffer until all fractions are collected (up to 26 fractions)

Sequencing Circulating Small RNA from Clinical Samples
Isolate total RNA from single fractions using the TRIZOL® isolation method
Measure RNA on BIOANALYZER® and use RT-PCR for quality control
Prepare small RNA library using manufacturer's guidelines (Illumina TRUSEQ®)
Sequence library using manufacturer's instructions (Illumina HISEQ®)

Plasma obtained from a patient with classic Hodgkin's lymphoma was subjected to SEC as described above. As shown in FIGS. 14A-14E, SEC allows the single-step isolation of circulating EVs from plasma. The miRNA distribution differs between the EV fraction and the protein/HDL fraction (FIG. 14E).

Using the above method, multiple vesicle-associated miRNAs were discovered, as well as protein fraction-associated miRNAs that allow therapy response monitoring in Hodgkin's patients. These results are discussed in Example 5.

### Example 5

### EVs Isolated from the Plasma of Hodgkin's Lymphoma

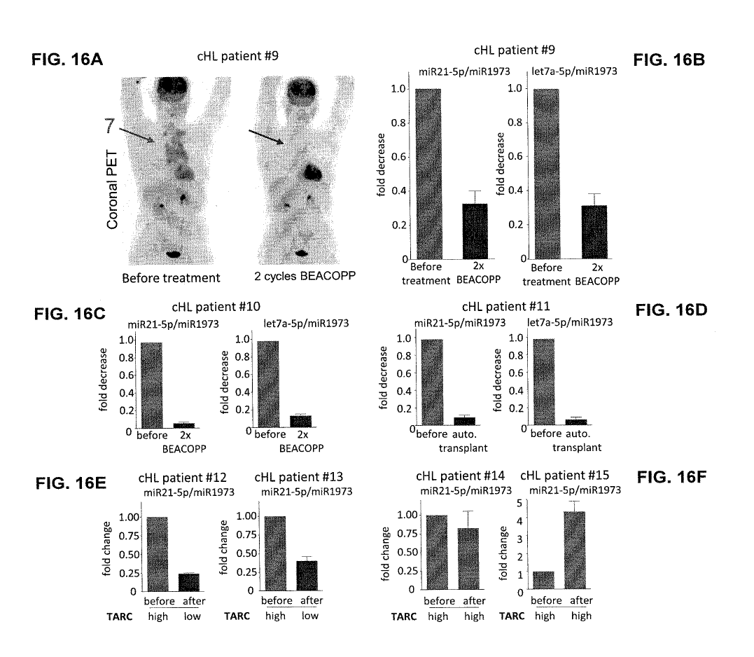

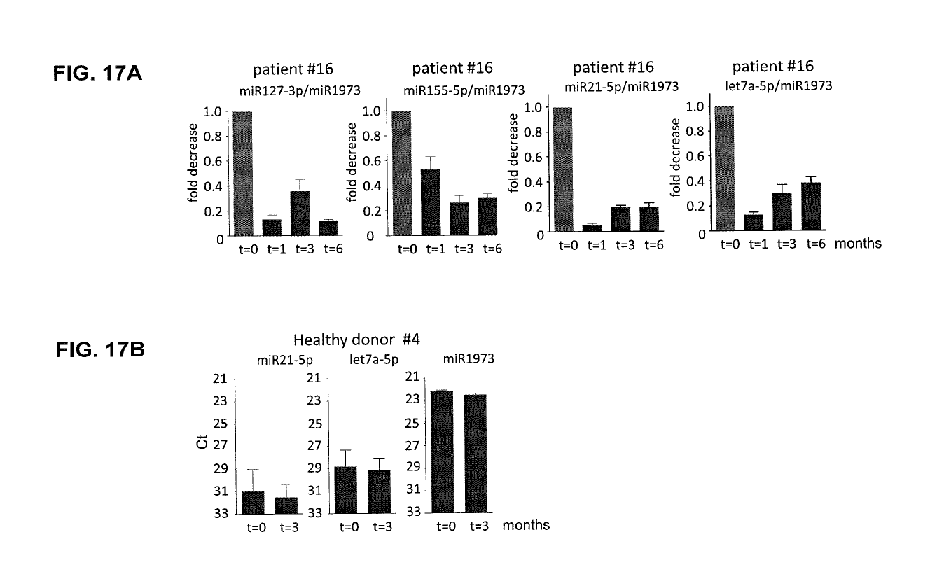

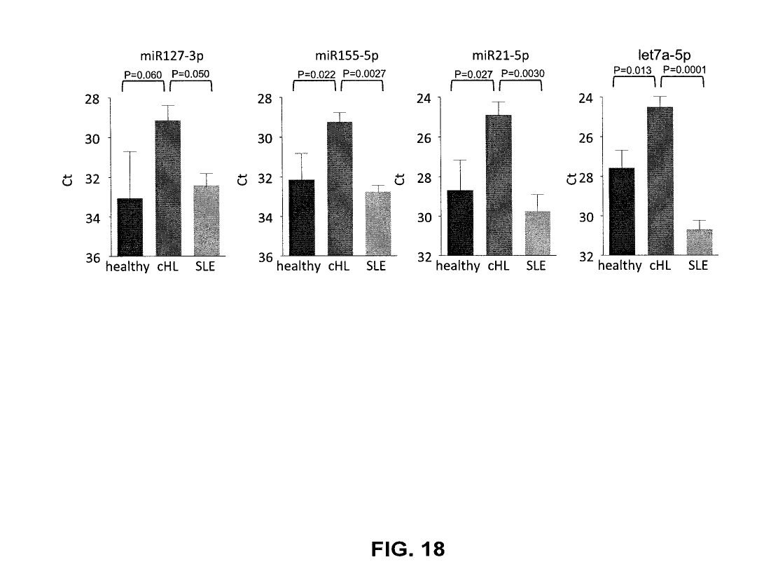

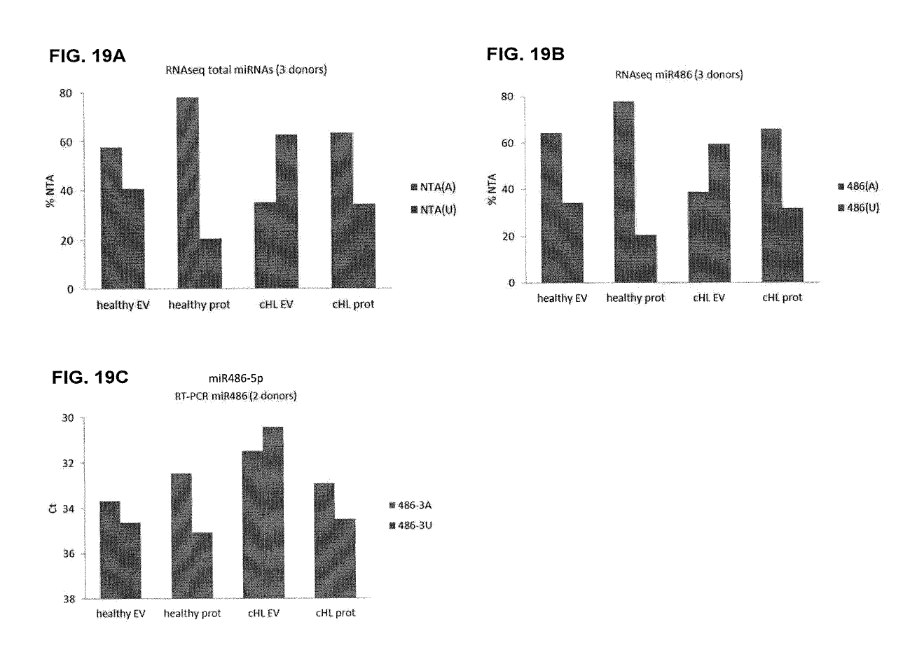

Plasma obtained from a patient with Hodgkin's lymphoma was subjected to SEC as described in Example 4. The results are presented in FIGS. 16A-16F, 17A, 17B, and 18.

### Example 6

### IsomiRs in Biofluids Discriminate Healthy Individuals from Hodgkin's Lymphoma Patients

Isolation of extracellular vesicles and protein-bound RNA fractions has been carried out as described in Examples 4 and 5. Expression profiling and data analysis of small ncRNAs were performed as in Examples 1 and 2.

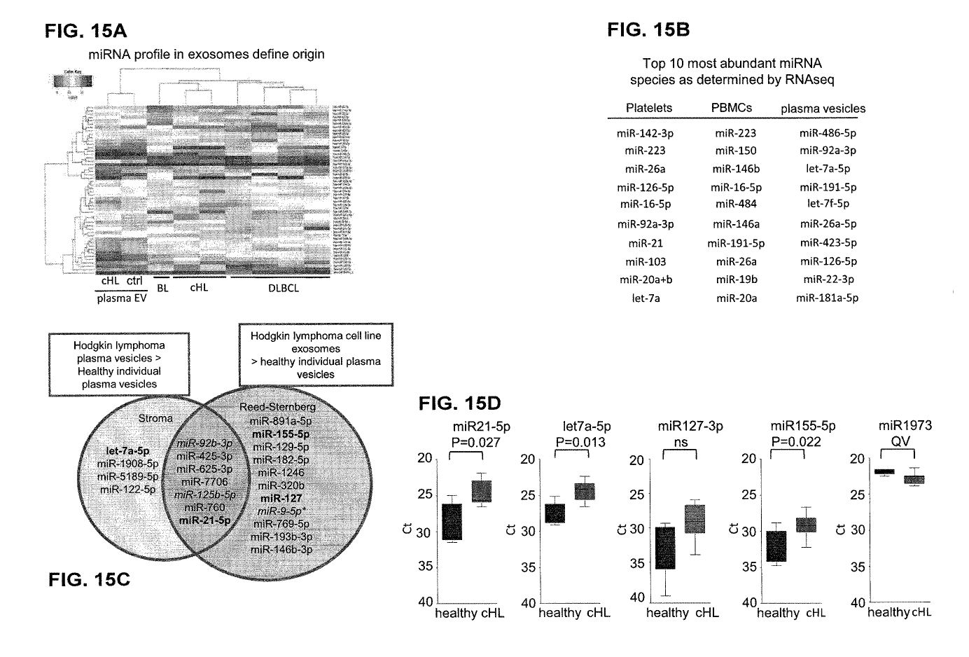

After detecting all reads that belong to a given miRNA, the reads are hierarchically classified (assigned to an isomiR type): i) the canonical miRNA sequence, ii) non-templated additions (NTA), and iii) 5′ and/or 3′ sequence variants (e.g., truncations or elongations) from the canonical sequence. The isomiR ratios are calculated for each miRNA and isomiR type, and are defined as the number of reads that belong to a given isomiR type divided by the total number of reads mapped to a given miRNA (canonical read count plus all isomiRs). The most statistically significant ratios (determined by p-value) are depicted in FIGS. 15A-15D. The ratio of variant to canonical is significant between healthy individuals and those having Hodgkin's lymphoma.

### Example 7

### IsomiRs in Biofluids Discriminate Breast Cancer Stage II from Stage III of Disease and Breast Cancer Patients with Disease Relapse from No-Relapse

Data source and experimental procedures are publicly available and accessible on the World Wide Web at ncbi.nlm.nih.gov/sra?term=SRP027589 and ncbi.nlm.nih.gov/bioproject?LinkName=sra_bioproject&from_uid=455584.

Publicly available sequence data of ncRNA in sera was analyzed to determine whether the ratio of ncRNA variants could be used to classify health status. Expression profiling and data analysis of small ncRNAs was performed as in Example 2.

After detecting all reads that belong to a given miRNA, the reads are hierarchically classified (assigned to an isomiR type): i) the canonical miRNA sequence, ii) non-templated additions (NTA), and iii) 5′ and/or 3′ sequence variants (e.g., truncations or elongations) from the canonical sequence. The isomiR ratios are calculated for each miRNA and isomiR type, and defined as the number of reads that belong to a given isomiR type divided by the total number of reads mapped to a given miRNA (canonical read count plus all isomiRs). The most statistically significant ratios (determined by p-value) are depicted in Table 6. The ratio of variant to canonical is significant between stage II patients and those having stage III breast cancer.

### Example 8

### IsomiRs in Tissue Discriminate Non-Tumor Adjacent Tissue from Testicular Germ Cell Tumors

Data source and experimental procedures are publicly available and accessible on the World Wide Web at ncbi.nlm.nih.gov/sra?term=SRP007946 and ncbi.nlm.nih.gov/bioproject/154993. Publicly available sequence data of ncRNA in sera was analyzed to determine whether the ratio of ncRNA variants could be used to classify health status.

Expression Profiling and Data Analysis of Small ncRNAs was Performed as in Example 2.

After detecting all reads that belong to a given miRNA, the reads are hierarchically classified (assigned to an isomiR type): i) the canonical miRNA sequence, ii) non-templated additions (NTA), and iii) 5′ and/or 3′ sequence variants (e.g., truncations or elongations) from the canonical sequence. The isomiR ratios are calculated for each miRNA and isomiR type, and defined as the number of reads that belong to a given isomiR type divided by the total number of reads mapped to a given miRNA (canonical read count plus all isomiRs). The most statistically significant ratios (determined by p-value) are depicted in Table 7. The ratio of variant to canonical is significant between adjacent tissue and tissue from testicular germ cell tumors.

### Example 9

### IsomiRs in Tissue Discriminate Colorectal Normal Tissue from Colorectal Tumor Tissue and from Colorectal Cancer-Related Metastasis Tissues

Data source and experimental procedures are publicly available and accessible on the World Wide Web at ncbi.nlm.nih.gov/Traces/sra/?study=SRP022054 and ncbi.nlm.nih.gov/bioproject/PRJNA20124S and C. Röhr et al., “High-throughput miRNA and mRNA sequencing of paired colorectal normal, tumor and metastasis tissues and bioinformatic modeling of miRNA-1 therapeutic applications,” PLoS One 2013 Jul. 2; 8(7):e67461. Publicly available sequence data of ncRNA in sera was analyzed to determine whether the ratio of ncRNA variants could be used to classify health status.

Expression Profiling and Data Analysis of Small ncRNAs was Performed as in Example 2.

After detecting all reads that belong to a given miRNA, the reads are hierarchically classified (assigned to an isomiR type): i) the canonical miRNA sequence, ii) non-templated additions (NTA), and iii) 5′ and/or 3′ sequence variants (e.g., truncations or elongations) from the canonical sequence. The isomiR ratios are calculated for each miRNA and isomiR type, and defined as the number of reads that belong to a given isomiR type divided by the total number of reads mapped to a given miRNA (canonical read count plus all isomiRs). The most statistically significant ratios (determined by p-value) are depicted in Table 8. The ratio of variant to canonical is significant between normal colon tissue and tissue from colorectal tumors, as well as between normal colon tissue and colorectal tumor-related metastasis as well as between colorectal tumor tissue and colorectal tumor-related metastasis.

### Example 10

### IsomiRs in Biofluids Discriminate Healthy Individuals from Patients Diagnosed with Alzheimer's Disease

Data source and experimental procedures are publicly available and accessible on the World Wide Web at ncbi.nlm.nih.gov/sra?term=SRP022043 and ncbi.nlm.nih.gov/bioproject?LinkName=sra_bioproject&from_uid=387967 and P. Leidinger et al., “A blood based 12-miRNA signature of Alzheimer disease patients,” Genome Biol. 2013 Jul. 29; 14(7):R78.

Publicly available sequence data of ncRNA in sera was analyzed to determine whether the ratio of ncRNA variants could be used to classify health status. Expression profiling and data analysis of small ncRNAs was performed as in Example 2.

After detecting all reads that belong to a given miRNA, the reads are hierarchically classified (assigned to an isomiR type): i) the canonical miRNA sequence, ii) non-templated additions (NTA), and iii) 5′ and/or 3′ sequence variants (e.g., truncations or elongations) from the canonical sequence. The isomiR ratios are calculated for each miRNA and isomiR type, and defined as the number of reads that belong to a given isomiR type divided by the total number of reads mapped to a given miRNA (canonical read count plus all isomiRs). The most statistically significant ratios (determined by p-value) are depicted in Table 9. The ratio of variant to canonical is significant between healthy individuals and those having Alzheimer's disease.

### REFERENCES

Amaral, P. P., M. E. Dinger, T. R. Mercer, and J. S. Mattick (2008). The eukaryotic genome as an RNA machine. Science 319:1787-1789.
Ameres, S. L., and P. D. Zamore (2013). Diversifying microRNA sequence and function. Nat. Rev. Mol. Cell Biol. 14:475-488.
Ameres, S. L., J. Martinez, and R. Schroeder (2007). Molecular basis for target RNA recognition and cleavage by human RISC. Cell 130:101-112.
Ameres, S. L., M. D. Horwich, J.-H. Hung, J. Xu, M. Ghildiyal, Z. Weng, and P. D. Zamore (2010). Target RNA-directed trimming and tailing of small silencing RNAs. Science 328:1534-1539.
Baccarini, A., H. Chauhan, T. J. Gardner, A. D. Jayaprakash, R. Sachidanandam, and B. D. Brown (2011). Kinetic analysis reveals the fate of a microRNA following target regulation in mammalian cells. Curr. Biol. 21:369-376.
Backes, S., J. S. Shapiro, L. R. Sabin, A. M. Pham, I. Reyes, B. Moss, S. Cherry, and B. R. tenOever (2012). Degradation of host microRNAs by poxvirus poly(A) polymerase reveals terminal RNA methylation as a protective antiviral mechanism. Cell Host Microbe 12:200-210.
Balaj, L., R. Lessard, L. Dai, Y.-J. Cho, S. L. Pomeroy, X. O. Breakefield, and J. Skog (2011). Tumor microvesicles contain retrotransposon elements and amplified oncogene sequences. Nat. Commun. 2:180.
Van Balkom, B. W. M., O. G. de Jong, M. Smits, J. Brummelman, K. den Ouden, P. M. de Bree, M. A. J. van Eijndhoven, D. M. Pegtel, W. Stoorvogel, and T. Würdinger et al. (2013). Endothelial cells require miR-214 to secrete exosomes that suppress senescence and induce angiogenesis in human and mouse endothelial cells. Blood 121:3997-4006, S1-15.
Bellingham, S. A., B. M. Coleman, and A. F. Hill (2012). Small RNA deep sequencing reveals a distinct miRNA signature released in exosomes from prion-infected neuronal cells. Nucleic Acids Res. 40:10937-10949.
Bijnsdorp, I. V., A. A. Geldof, M. Lavaei, S. R. Piersma, R. J. A. van Moorselaar, and C. R. Jimenez (2013). Exosomal ITGA3 interferes with non-cancerous prostate cell functions and is increased in urine exosomes of metastatic prostate cancer patients. J. Extracell. Vesicles 2.
Burns, D. M., A. D'Ambrogio, S. Nottrott, and J. D. Richter (2011). CPEB and two poly(A) polymerases control miR-122 stability and p53 mRNA translation. Nature 473:105-108.
Burroughs, A. M., Y. Ando, M. J. L. de Hoon, Y. Tomaru, T. Nishibu, R. Ukekawa, T. Funakoshi, T. Kurokawa, H. Suzuki, and Y. Hayashizaki, et al. (2010). A comprehensive survey of 3′ animal miRNA modification events and a possible role for 3′ adenylation in modulating miRNA targeting effectiveness. Genome Res. 20:1398-1410.
Chairoungdua, A., D. L. Smith, P. Pochard, M. Hull, and M. J. Caplan (2010). Exosome release of β-catenin: a novel mechanism that antagonizes Wnt signaling. J. Cell. Biol. 190:1079-1091.
Chitwood, D. H., and M. C. P. Timmermans (2010). Small RNAs are on the move. Nature 467:415-419.
Chuma, S., and R. S. Pillai (2009). Retrotransposon silencing by piRNAs: ping-pong players mark their sub-cellular boundaries. PLoS Genet. 5:e1000770.
Cullen, B. R. (2009). Viral and cellular messenger RNA targets of viral microRNAs. Nature 457:421-425.
D'Ambrogio, A., W. Gu, T. Udagawa, C. C. Mello, and J. D. Richter (2012). Specific miRNA stabilization by Gld2-catalyzed monoadenylation. Cell Rep. 2:1537-1545.
Ebert, M. S., and P. A. Sharp (2012). Roles for microRNAs in conferring robustness to biological processes. Cell 149:515-524.
Feederle, R., J. Haar, K. Bernhardt, S. D. Linnstaedt, H. Bannert, H. Lips, B. R. Cullen, and H.-J. Delecluse (2011). The members of an Epstein-Barr virus microRNA cluster cooperate to transform B lymphocytes. J. Virol. 85:9801-9810.
Fernandez-Valverde, S. L., R. J. Taft, and J. S. Mattick (2010). Dynamic isomiR regulation in Drosophila development. RNA 16:1881-1888.
Garcia, E. L., A. Onafuwa-Nuga, S. Sim, S. R. King, S. L. Wolin, and A. Telesnitsky (2009). Packaging of host mY RNAs by murine leukemia virus may occur early in Y RNA biogenesis. J. Virol. 83:12526-12534.
Gibbings, D. J., C. Ciaudo, M. Erhardt, and O. Voinnet (2009). Multi-vesicular bodies associate with components of miRNA effector complexes and modulate miRNA activity. Nat. Cell. Biol. 11:1143-1149.
Guasparri, I., D. Bubman, and E. Cesarman (2008). EBV LMP2A affects LMP1-mediated NF-kappaB signaling and survival of lymphoma cells by regulating TRAF2 expression. Blood 111:3813-3820.
Guduric-Fuchs, J., A. O'Connor, B. Camp, C. L. O'Neill, R. J. Medina, and D. A. Simpson (2012). Selective extracellular vesicle-mediated export of an overlapping set of microRNAs from multiple cell types. BMC Genomics 13:357.
Guo, L., Q. Yang, J. Lu, H. Li, Q. Ge, W. Gu, Y. Bai, and Z. Lu (2011). A comprehensive survey of miRNA repertoire and 3′ addition events in the placentas of patients with pre-eclampsia from high-throughput sequencing. PLoS One 6:e21072.
Jones, M. R., L. J. Quinton, M. T. Blahna, J. R. Neilson, S. Fu, A. R. Ivanov, D. A. Wolf, and J. P. Mizgerd (2009). Zcchc11-dependent uridylation of microRNA directs cytokine expression. Nat. Cell. Biol. 11:1157-1163.
Kai, Z. S., and A. E. Pasquinelli (2010). MicroRNA assassins: factors that regulate the disappearance of miRNAs. Nat. Struct. Mol. Biol. 17:5-10.
Katoh, T., Y. Sakaguchi, K. Miyauchi, T. Suzuki, S.-I. Kashiwabara, T. Baba, and T. Suzuki (2009). Selective stabilization of mammalian microRNAs by 3′ adenylation mediated by the cytoplasmic poly(A) polymerase GLD-2. Genes Dev. 23:433-438.
Khan, M. A., R. Goila-Gaur, S. Opi, E. Miyagi, H. Takeuchi, S. Kao, and K. Strebel (2007). Analysis of the contribution of cellular and viral RNA to the packaging of APOBEC3G into HIV-1 virions. Retrovirology 4:48.
Koppers-Lalic, D., M. M. Hogenboom, J. M. Middeldorp, and D. M. Pegtel (2012). Virus-modified exosomes for targeted ma delivery; A new approach in nanomedicine. Adv. Drug Deliv. Rev.
Kosaka, N., H. Iguchi, Y. Yoshioka, F. Takeshita, Y. Matsuki, and T. Ochiya (2010). Secretory mechanisms and intercellular transfer of microRNAs in living cells. J. Biol. Chem. 285:17442-17452.
Kosaka, N., H. Iguchi, K. Hagiwara, Y. Yoshioka, F. Takeshita, and T. Ochiya (2013). Neutral sphingomyelinase 2 (nSMase2)-dependent exosomal transfer of angiogenic microRNAs regulate cancer cell metastasis. J. Biol. Chem. 288:10849-10859.
Lee, Y. S., S. Pressman, A. P. Andress, K. Kim, J. L. White, J. J. Cassidy, X. Li, K. Lubell, D. H. Lim, and I. S. Cho et al. (2009a). Silencing by small RNAs is linked to endosomal trafficking. Nat. Cell Biol. 11:1150-1156.
Lee, Y. S., Y. Shibata, A. Malhotra, and A. Dutta (2009b). A novel class of small RNAs: tRNA-derived RNA fragments (tRFs). Genes Dev. 23:2639-2649.
Lujambio, A., and M. Esteller (2007). CpG island hypermethylation of tumor suppressor microRNAs in human cancer. Cell Cycle 6:1455-1459.
Martens-Uzunova, E. S., M. Olvedy, and G. Jenster (2013). Beyond microRNA-novel RNAs derived from small non-coding RNA and their implication in cancer. Cancer Lett. 340:201-211.
Martin, G., and W. Keller (2007). RNA-specific ribonucleotidyl transferases. RNA 13:1834-1849.
Maute, R. L., C. Schneider, P. Sumazin, A. Holmes, A. Califano, K. Basso, and R. Dalla-Favera (2013). tRNA-derived microRNA modulates proliferation and the DNA damage response and is down-regulated in B cell lymphoma. Proc. Natl. Acad. Sci. U.S. A. 110:1404-1409.
Mittelbrunn, M., and F. Sánchez-Madrid (2012). Intercellular communication: diverse structures for exchange of genetic information. Nat. Rev. Mol. Cell Biol. 13:328-335.
Mittelbrunn, M., C. Gutiérrez-Vázquez, C. Villarroya-Beltri, S. González, F. Sánchez-Cabo, M. A. González, A. Bernad, and F. Sänchez-Madrid (2011). Unidirectional transfer of microRNA-loaded exosomes from T cells to antigen-presenting cells. Nat. Commun. 2:282.
Montecalvo, A., A. T. Larregina, W. J. Shufesky, D. B. Stolz, M. L. G. Sullivan, J. M. Karlsson, C. J. Baty, G. A. Gibson, G. Erdos, and Z. Wang, et al. (2012). Mechanism of transfer of functional microRNAs between mouse dendritic cells via exosomes. Blood 119:756-766.
Morin, R. D., M. D. O'Connor, M. Griffith, F. Kuchenbauer, A. Delaney, A.-L. Prabhu, Y. Zhao, H. McDonald, T. Zeng, and M. Hirst, et al. (2008). Application of massively parallel sequencing to microRNA profiling and discovery in human embryonic stem cells. Genome Res. 18:610-621.
Mullokandov, G., A. Baccarini, A. Ruzo, A. D. Jayaprakash, N. Tung, B. Israelow, M. J. Evans, R. Sachidanandam, and B. D. Brown (2012). High-throughput assessment of microRNA activity and function using microRNA sensor and decoy libraries. Nat. Methods 9:840-846.
Nolte-'t Hoen, E. N. M., H. P. J. Buermans, M. Waasdorp, W. Stoorvogel, M. H. M. Wauben, and P. A. C.'t Hoen (2012). Deep sequencing of RNA from immune cell-derived vesicles uncovers the selective incorporation of small non-coding RNA biotypes with potential regulatory functions. Nucleic Acids Res. 40:9272-9285.
Palma, J., S. C. Yaddanapudi, L. Pigati, M. A. Havens, S. Jeong, G. A. Weiner, K. M. E. Weimer, B. Stern, M. L. Hastings, and D. M. Duelli (2012). MicroRNAs are exported from malignant cells in customized particles. Nucleic Acids Res. 40:9125-9138.
Parekh, S., J. M. Polo, R. Shaknovich, P. Juszczynski, P. Lev, S. M. Ranuncolo, Y. Yin, U. Klein, G. Cattoretti, and R. Dalla Favera, et al. (2007). BCL6 programs lymphoma cells for survival and differentiation through distinct biochemical mechanisms. Blood 110:2067-2074.
Pegtel, D. M., K. Cosmopoulos, D. A. Thorley-Lawson, M. A. J. van Eijndhoven, E. S. Hopmans, J. L. Lindenberg, T. D. de Gruijl, T. Würdinger, and J. M. Middeldorp (2010). Functional delivery of viral miRNAs via exosomes. Proc. Natl. Acad. Sci. U.S. A. 107:6328-6333.
Pegtel, D. M., M. D. B. van de Garde, and J. M. Middeldorp (2011). Viral miRNAs exploiting the endosomal-exosomal pathway for intercellular cross-talk and immune evasion. Biochim. Biophys. Acta 1809:715-721.
Polikepahad, S., and D. B. Corry (2013). Profiling of T helper cell-derived small RNAs reveals unique antisense transcripts and differential association of miRNAs with argonaute proteins 1 and 2. Nucleic Acids Res. 41:1164-1177.
Qiu, J., K. Cosmopoulos, M. Pegtel, E. Hopmans, P. Murray, J. Middeldorp, M. Shapiro, and D. A. Thorley-Lawson (2011). A novel persistence associated EBV miRNA expression profile is disrupted in neoplasia. PLoS Pathog. 7:e1002193.
Riley, K. J., G. S. Rabinowitz, T. A. Yario, J. M. Luna, R. B. Darnell, and J. A. Steitz (2012). EBV and human microRNAs co-target oncogenic and apoptotic viral and human genes during latency. EMBO J. 31:2207-2221.
Robinson, M. D., D. J. McCarthy, and G. K. Smyth (2010). edgeR: a Bioconductor package for differential expression analysis of digital gene expression data. Bioinformatics 26:139-140.
Routh, A., T. Domitrovic, and J. E. Johnson (2012). Host RNAs, including transposons, are encapsidated by a eukaryotic single-stranded RNA virus. Proc. Natl. Acad. Sci. U.S. A. 109:1907-1912.
Rüegger, S., and H. Groβhans (2012). MicroRNA turnover: when, how, and why. Trends Biochem. Sci. 37:436-446.
Saito, Y., G. Liang, G. Egger, J. M. Friedman, J. C. Chuang, G. A. Coetzee, and P. A. Jones (2006). Specific activation of microRNA-127 with downregulation of the proto-oncogene BCL6 by chromatin-modifying drugs in human cancer cells. Cancer Cell. 9:435-443.
Scott, D. D., and C. J. Norbury. RNA decay via 3′ uridylation. Biochim. Biophys. Acta 1829:654-665.
Scott, D. D., and C. J. Norbury (2013). RNA decay via 3′ uridylation. Biochim. Biophys. Acta Gene Regul. Mech. 1829:654-665.
Seto, E., A. Moosmann, S. Gromminger, N. Walz, A. Grundhoff, and W. Hammerschmidt (2010). Micro RNAs of Epstein-Barr virus promote cell cycle progression and prevent apoptosis of primary human B cells. PLoS Pathog. 6:e1001063.
Sim, S., D. E. Weinberg, G. Fuchs, K. Choi, J. Chung, and S. L. Wolin (2009). The subcellular distribution of an RNA quality control protein, the Ro autoantigen, is regulated by noncoding Y RNA binding. Mol. Biol. Cell 20:1555-1564.
Skalsky, R. L., D. L. Corcoran, E. Gottwein, C. L. Frank, D. Kang, M. Hafner, J. D. Nusbaum, R. Feederle, H.-J. Delecluse, and M. A. Luftig, et al. (2012). The viral and cellular microRNA targetome in lymphoblastoid cell lines. PLoS Pathog. 8:e1002484.
Song, L., C. Lin, H. Gong, C. Wang, L. Liu, J. Wu, S. Tao, B. Hu, S.-Y. Cheng, and M. Li, et al. (2013). miR-486 sustains NF-κB activity by disrupting multiple NF-κB-negative feedback loops. Cell Res. 23:274-289.
Umezu, T., K. Ohyashiki, M. Kuroda, and J. H. Ohyashiki (2013). Leukemia cell to endothelial cell communication via exosomal miRNAs. Oncogene 32:2747-2755.
Vereide, D. T., E. Seto, Y.-F. Chiu, M. Hayes, T. Tagawa, A. Grundhoff, W. Hammerschmidt, and B. Sugden (2013). Epstein-Barr virus maintains lymphomas via its miRNAs. Oncogene.
Verweij, F. J., M. A. J. van Eijndhoven, J. Middeldorp, and D. M. Pegtel (2013). Analysis of viral microRNA exchange via exosomes in vitro and in vivo. Methods Mol. Biol. 1024:53-68.
Villarroya-Beltri, C., C. Gutiérrez-Vázquez, F. Sánchez-Cabo, D. Pérez-Hernández, J. Vázquez, N. Martin-Cofreces, D. J. Martinez-Herrera, A. Pascual-Montano, M. Mittelbrunn, and F. Sánchez-Madrid (2013). Sumoylated hnRNPA2B1 controls the sorting of miRNAs into exosomes through binding to specific motifs. Nat. Commun. 4:2980.
Wee, L. M., C. F. Flores-Jasso, W. E. Salomon, and P. D. Zamore (2012). Argonaute divides its RNA guide into domains with distinct functions and RNA-binding properties. Cell 151:1055-1067.
Wyman, S. K., E. C. Knouf, R. K. Parkin, B. R. Fritz, D. W. Lin, L. M. Dennis, M. A. Krouse, P. J. Webster, and M. Tewari (2011). Post-transcriptional generation of miRNA variants by multiple nucleotidyl transferases contributes to miRNA transcriptome complexity. Genome Res. 21:1450-1461.
Yao, B., L. B. La, Y.-C. Chen, L.-J. Chang, and E. K. L. Chan (2012). Defining a new role of GW182 in maintaining miRNA stability. EMBO Rep. 13:1102-1108.
Zhang, Y., D. Liu, X. Chen, J. Li, L. Li, Z. Bian, F. Sun, J. Lu, Y. Yin, X. Cai, et al. (2010). Secreted monocytic miR-150 enhances targeted endothelial cell migration. Mol. Cell 39:133-144.
Zhuang, G., X. Wu, Z. Jiang, I. Kasman, J. Yao, Y. Guan, J. Oeh, Z. Modrusan, C. Bais, and D. Sampath, et al. (2012). Tumor-secreted miR-9 promotes endothelial cell migration and angiogenesis by activating the JAK-STAT pathway. EMBO J. 31:3513-3523.

1. A method for identifying a small non-coding RNA (ncRNA) biomarker pair, the method comprising
determining the quantity of two different varieties of said ncRNA in a first bodily fluid sample obtained from a first individual reference and in a second bodily fluid sample obtained from a second individual reference, wherein said first individual reference has an altered health status from said second individual reference,
determining the ratio of the two different varieties in the first bodily fluid sample,
determining the ratio of the two different varieties in the second bodily fluid sample,
comparing the ratios from said first and second bodily fluid sample, and
identifying the two different varieties of said ncRNA as a biomarker pair when the ratio is altered between said first and second bodily fluid sample,
wherein the first and second varieties are selected from the canonical ncRNA; the ncRNA trimmed at the 5′ or 3′ end and/or extended at the 5′ or 3′ end; and the ncRNA with a 3′ non-templated nucleotide addition, optionally trimmed at the 5′ or 3′ end or extended at the 5′ or 3′ end;
wherein when said ncRNA is not an miRNA the first variety is selected from the canonical ncRNA and the ncRNA with a 3′ non-templated nucleotide addition and the second variety is the ncRNA with a 3′ non-templated nucleotide addition.

2.-4. (canceled)

5. A method for collecting data on the health status of an individual utilizing a small non-coding RNA (ncRNA) biomarker pair, the method comprising:
determining the quantity of the biomarker pair in a bodily fluid sample from the individual and determining the ratio of the biomarker pair in the bodily fluid sample,
wherein the biomarker pair consists of two different varieties of an ncRNA
wherein the first and second varieties are selected from the canonical ncRNA; the ncRNA trimmed at the 5′ or 3′ end and/or extended at the 5′ or 3′ end; and the ncRNA with a 3′ non-templated nucleotide addition, optionally trimmed at the 5′ or 3′ end or extended at the 5′ or 3′ end;
wherein when said ncRNA is not an miRNA the first variety is selected from the canonical ncRNA and the ncRNA with a 3′ non-templated nucleotide addition and the second variety is the ncRNA with a 3′ non-templated nucleotide addition.

6.-16. (canceled)

17. A method for characterizing the status of lymphoma in an individual, the method comprising:
determining the quantity of one or more miRNAs from a bodily fluid sample from the individual and comparing the amount of the one or more miRNAs to a reference sample,
wherein the difference in the presence or amount of the one or more miRNAs characterizes the status of the individual, and wherein the one or more miRNAs are selected from the group consisting of let-7a-5p, miR-1908-5p, miR-5189-5p, miR-92b-3p, miR-425-3p, miR-625-3p, miR-7706, miR-125b-5p, miR-760, miR-21-5p, miR-122-5p, miR-891a-5p, miR-155-5p, miR-129-5p, miR-182-5p, miR-1246, miR-320b, miR-127-3p, miR-9-5p, miR-769-5p, miR-193b-3p, and miR-146b-3p.

18.-31. (canceled)

32. A method for collecting data on the health status of an individual, the method comprising:
purifying exosomal vesicles from a bodily fluid sample from the individual,
determining the quantity of the one or more miRNAs associated with the purified exosomal vesicles, and
comparing the amount of the one or more miRNAs to a reference sample,
wherein the one or more miRNAs are selected from the group consisting of miR-127-3p, let-7a-5p, miR-1908-5p, miR-5189-5p, miR-92b-3p, miR-425-3p, miR-625-3p, miR-7706, miR-125b-5p, miR-760, miR-21-5p, miR-122-5p, miR-891a-5p, miR-155-5p, miR-129-5p, miR-182-5p, miR-1246, miR-320b, miR-9-5p, miR-769-5p, miR-193b-3p, and miR-146b-3p.

33. The method of claim 32, wherein the one or more miRNAs are selected from the group consisting of miR-127-3p, miR-21-5p, let-7a-5p, and miR-155-5p.

34. The method of claim 32, wherein the reference sample is from one or more healthy individuals.

35. The method of claim 32, wherein the reference sample is from one or more individuals having classical Hodgkin's lymphoma (cHL).

36. The method of claim 32, wherein the reference sample is from the same individual prior to receiving treatment for classical Hodgkin's lymphoma (cHL).

37. The method of claim 32, wherein the reference sample is from at least one individual having a good response to treatment for a disorder or from at least one individual having a poor response to treatment for a disorder.

38. The method of claim 32, wherein the exosomal vesicles are purified by subjecting the bodily fluid samples to size-exclusion chromatography (SEC).

39. The method of claim 38, wherein the exosomal vesicles are isolated by obtaining the void volume fraction.

40. The method of claim 32, wherein the bodily fluid is blood or urine.

41. A method for treating lymphoma in an individual, the method comprising:
a) purifying exosomal vesicles from a first bodily fluid sample from the individual and determining the quantity of one or more miRNAs associated with the purified exosomal vesicles, wherein the one or more miRNAs are selected from the group consisting of miR-127-3p, let-7a-5p, miR-1908-5p, miR-5189-5p, miR-92b-3p, miR-425-3p, miR-625-3p, miR-7706, miR-125b-5p, miR-760, miR-21-5p, miR-122-5p, miR-891a-5p, miR-155-5p, miR-129-5p, miR-182-5p, miR-1246, miR-320b, miR-9-5p, miR-769-5p, miR-193b-3p, and miR-146b-3p,
b) purifying exosomal vesicles from a second bodily fluid sample from the individual and determining the quantity of one or more miRNAs associated with the purified exosomal vesicles, and
c) providing a lymphoma treatment to an individual having an altered amount of the one or more miRNAs between the first and second bodily fluid sample.

42. The method of claim 41, wherein the lymphoma is classical Hodgkin's lymphoma (cHL).

43. A method for treating relapsed classical Hodgkin's lymphoma (cHL) in an individual, the method comprising:
a) purifying exosomal vesicles from a first bodily fluid sample from the individual following a treatment for cHL and determining the quantity of one or more miRNAs associated with the purified exosomal vesicles, wherein the one or more miRNAs are selected from the group consisting of miR-12′7-3p, let-7a-5p, miR-1908-5p, miR-5189-5p, miR-92b-3p, miR-425-3p, miR-625-3p, miR-7706, miR-125b-5p, miR-760, miR-21-5p, miR-122-5p, miR-891a-5p, miR-155-5p, miR-129-5p, miR-182-5p, miR-1246, miR-320b, miR-9-5p, miR-769-5p, miR-193b-3p, and miR-146b-3p, followed by
b) purifying exosomal vesicles from a second bodily fluid sample from the individual and determining the quantity of the one or more miRNAs associated with the purified exosomal vesicles, and
c) treating an individual having a higher amount of the one or more miRNAs in the second sample as compared to the first sample for relapsed cHL.

44. The method of claim 43, wherein the one or more miRNAs are selected from the group consisting of miR-21-5p, let-7a-5p, miR-127-3p, and miR-155-5p.

45. The method of claim 43, wherein the exosomal vesicles are purified by subjecting the bodily fluid samples to size-exclusion chromatography (SEC).

46. The method of claim 45, wherein the exosomal vesicles are isolated by obtaining the void volume fraction.

47. The method of claim 43, wherein the bodily fluid is blood or urine.
